(19) United States
(12) Patent Application Publication
(10) Pub. No.: US 2024/0102094 A1
(43) Pub. Date: Mar. 28, 2024

(54) TREM2 AGONIST BIOMARKERS AND METHODS OF USE THEREOF

(71) Applicant: AMGEN INC., Thousand Oaks, CA (US)

(72) Inventors: Daniel C. ELLWANGER, Thousand Oaks, CA (US); Ian N. FOLTZ, Thousand Oaks, CA (US); Samuel A. HASSON, Thousand Oaks, CA (US)

(73) Assignee: AMGEN INC., Thousand Oaks, CA (US)

(21) Appl. No.: 18/255,524

(22) PCT Filed: Dec. 3, 2021
(86) PCT No.: PCT/US2021/072719

§ 371 Date: Jun. 1, 2023

Related U.S. Application Data
(60) Provisional application No. 63/262,943, filed on Oct. 22, 2021.
(60) Provisional application No. 63/120,986, filed on Dec. 3, 2020.

(51) Int. Cl. C12Q 1/6883; C12Q 1/6874; G01N 33/68

(52) U.S. Cl. CPC C12Q 1/6883; C12Q 1/6874; G01N 33/6896; C12Q 2600/106; C12Q 2600/158; G01N 2800/2821; G01N 2800/52

(57) ABSTRACT

The present invention provides a method of treating a disease or conditions associated with a dysfunction of TREM2 in a human patient, such as Alzheimer's disease, comprising administering to the patient a TREM2 agonist. In another aspect, the invention provides a method of assaying a biological sample taken from a patient having Alzheimer's for biomarkers to determine potential benefit or if the disease has an increased probability of responding to treatment with a TREM2 agonist.

### 1. SEQUENCE LISTING

The instant application contains a Sequence Listing which has been submitted electronically in ASCII format and is hereby incorporated by reference in its entirety. Said ASCII copy, created on Nov. 30, 2021, is named 004WO_SL_ST25.txt and is 28,469 bytes in size.

### 2. TECHNICAL FIELD OF THE INVENTION

The present invention relates generally to treatment of Alzheimer's disease (AD) using a TREM2 agonist. More specifically, the present invention relates to certain Alzheimer's disease biomarkers and/or certain TREM2 agonism biomarkers, as well as their use in methods for treating Alzheimer's disease, for example, in evaluating and/or predicting patient responses to treatment.

### 3. BACKGROUND

Alzheimer's disease (AD) is the most common cause of progressive dementia in older adults affecting more than 5.5 million Americans and representing the 6th leading cause of death in the United States.

Microglia are brain-resident macrophages with many homeostatic and injury responsive roles, including trophic and phagocytic functions. Microglial responses that modulate disease course are triggered by AD pathology. Pathological features of AD include extracellular amyloid plaques composed of the amyloid 6 (A6) peptide, intraneuronal neurofibrillary tangles consisting of aggregated, hyperphosphorylated tau protein, neuroimmune activation, and reductions in synaptic density (Long and Holtzman, Cell, 2019). Microglia accumulate around A6 plaques to contain and compact them, thereby reducing markers of axonal dystrophy in surrounding neurons (Yuan et al, Neuron, 2016). During this process, microglia modify their phenotypic and transcriptional properties, transitioning from a “homeostatic” to an activated profile, for example, towards a disease associated microglia trajectory (DAM) (Carmona et al., The genetic landscape of Alzheimer disease, 1st Ed., 2018).

Microglial transitions to activated profiles have been shown to depend on Triggering Receptor Expressed in Myeloid cells 2 (TREM2), a macrophage cell surface receptor abundantly expressed in microglia (Ulland and Colonna, Nat. Rev. Neurol., 2018). TREM2 sustains microglia response to brain injury stimuli including apoptotic cells, myelin damage, and amyloid β (Aβ). Due of its role in metabolic activation, TREM2 is believed to function as a costimulatory molecule that sustains microglial activation trajectories.

Activation of microglia through TREM2 may provide a promising therapeutic approach in the treatment of AD, however the impact of therapy using a TREM2 agonist in the context of microglial activation trajectories in Alzheimer's disease is not established, creating a significant unmet need.

### 4. SUMMARY

In one aspect, the present invention provides a method of treating a disease or conditions associated with a dysfunction of TREM2 in a human patient, comprising administering to the patient an effective amount of a TREM2 agonist to increase the activity of triggering receptor expressed on myeloid cells 2 (TREM2). In some embodiments, a method of treating Alzheimer's disease is provided.

In another aspect, the present invention provides a method of assaying a biological sample taken from a patient having Alzheimer's to determine potential benefit or if the disease has an increased probability of responding to treatment with a TREM2 agonist. Other aspects provide a method of evaluating and monitoring patient biomarker responses to TREM2 agonist therapy. In some embodiments, the TREM2 agonist biomarkers are selected from CCL4, CCL2, CST7, CXCL2, CXCL10, IL1B, and TMEM119.

In yet another aspect, the present invention provides a method of inducing microglial activation in a patient towards specific microglia cell type trajectories, for example towards an interferon-responsive (IFN-R), cycling (Cyc-M), or MHC-II expressing (MHC-II) type microglia trajectory, the method comprising administering to the patient an effective amount of a TREM2 agonist.

### 5. BRIEF DESCRIPTION OF THE DRAWINGS

FIGS. 1A-1J show properties of hTREM2 agonistic antibody hT2AB. FIG. 1A shows that hT2AB specifically binds to hTREM2 but does not bind hTREM1 or mTREM2. The data shows the binding signals of hTREM2-His to hT2AB measured by Octet. 1B12 is an anti-hTREM1 antibody that binds exclusively to hTREM1-His. There is no binding to either hTREM1-His or hTREM2-His by irrelevant hIgG2 (left panel). hT2AB binds hTREM2 transiently co-expressing hDAP12 in HEK293 cells (clone G13) but does not bind HEK293 cells that express mTREM2 and mDAP12 (Right panel). FIGS. 1B-1C show functional EC50 of hT2AB in clone G13 cells (FIG. 1B) and human monocyte-derived macrophages (hMacs) (FIG. 1C). Activation of hTREM2 was determined by measuring the induction of Syk phosphorylation after exposure of cells to different concentrations of hT2AB (n=8). FIG. 1D shows activation of hTREM2 by hT2AB IgG1 and hT2AB Fab, determined by measuring Syk phosphorylation in clone G13 cells exposed to different concentrations of antibodies (n=3). FIG. 1E shows quantification of sTREM2 in conditioned media from hMacs treated with different concentrations of hT2AB or control hIgG1 antibody for 24 hours without any immune challenge (n=4 for each group). FIG. 1F shows quantification of CCL4 in conditioned media from hMacs treated with hT2AB or control hIgG1 antibody at 200 nM for different time points. Acetylated LDL was used as a positive control (n=2 for each group). FIG. 1G shows a cell viability assay of bone marrow-derived macrophages (BMMs) from TREM2CV mice after CSF1 withdrawal treated with different concentrations of plate bound hT2AB or control hIgG1 for 48 hours (n=3 for each group). FIG. 1H shows a binding assay of hT2AB for BMMs from TREM2CV and TREM2R47H. BMMs from Trem2−/− were used as a negative control. FIG. 1I shows 2B4 reporter cell lines expressing hTREM2 (CV or R47H) stimulated with different amounts of plate bound hT2AB or control hIgG1. GFP expression was measured by flow cytometry (n=2 for each group). FIG. 1J shows a cell viability assay of BMMs from TREM2R47H mice after CSF1 withdrawal treated with plate bound hT2AB or control hIgG1 at 10 μg/mL for 48 hours. *, P<0.05; **, P<0.01 by two-way ANOVA with Sidak's multiple comparisons test. All data in FIG. 1 are shown as mean SD, except for FIGS. 1B-1C, that depict mean±SEM.

FIGS. 2A-2K show pharmacokinetics and pharmacodynamics of hT2AB. FIGS. 2A-2E depict pharmacodynamic (PD) studies of hT2AB in TREM2CV, TREM2R47H or Trem2−/− male mice. Different mouse cohorts received a single dose of hT2AB i.v. in the range of 0 (vehicle) to 100 mg/kg for 24 hours. Concentrations of chemokines, including CXCL10 (FIG. 2A), CCL4 (FIG. 2B), CCL2 (FIG. 2C) and CXCL2 (FIG. 2D), as well as a microglia activation biomarker CST7 (FIG. 2E) were measured by Meso Scale Discovery (MSD) technology in brain lysates (vehicle, n=4 for TREM2CV, n=5 for TREM2R47H or Trem2−/−; 1, 3 and 10 mg/kg, n=2 for TREM2CV or TREM2R47H; 30 mg/kg, n=5 for all three genotypes; 100 mg/kg, n=2 for TREM2CV or TREM2R47H, n=5 for Trem2−/−). FIGS. 1F-K depict a single dose of hT2AB administered i.v. in TREM2R47H or Trem2−/− male mice at 30 mg/kg. Different mouse cohorts were sacrificed after 4, 8 and 24 hours after antibody treatment and brain tissues were collected and lysed for measurement of hT2AB antibody levels and expression of neurodegeneration-associated microglial activation biomarkers (n=5 for each group). FIG. 2F shows hT2AB brain concentration (nM) is ˜25 fold higher than the EC50 values for Syk phosphorylation (222 pM) in clone G13 cells up to 24 hours after i.v. administration of 30 mg/kg hT2AB. qRT-PCR analysis shows increased expression of Cxcl10 (FIG. 2G), Ccl2 (FIG. 2H), Ccl4 (FIG. 2I), Cst7 (FIG. 2J), as well as the homeostatic microglia marker Tmem119 (K) upon hT2AB treatment by. *, P<0.05; **, P<0.01; ****, P<0.0001 by two-way ANOVA with Sidak's multiple comparisons test; all data are shown as mean±SD.

FIGS. 3A-3G show sampling of microglia from the human TREM2-tg-5XFAD Mouse Models. FIG. 3A shows protocol design. Two days after antibody injection, CD45+ cells were collected from cortex of male and female 5XFAD mice with endogenous Trem2 knock-out (Trem2−/−) with or without one of two variants of a human TREM2 knock-in: common variant (CV) and R47H. 71,303 cells passed scRNA-seq quality control. FIGS. 3B-3D show supervised immune cell type classification. Individual cells were assigned a similarity score to 830 microarray samples of sorted mouse immune cells generated by the Immunologic Genome Project. Cells were embedded in a lower-dimensional latent space while blocking observed covariates. Cell type labels were corrected by the enriched cell type of each segment of the latent space. FIG. 3E shows uniform manifold approximation and projection (UMAP) of all cells representing the global data structure; cells are colored by the 10 identified immune cell types were identified. FIG. 3F shows differential gene expression of microglia. Absolute differences in expression levels to other CD45+ cells are quantified by effect size; gene expression specificity and gene detection rate were determined using conditional probabilities with uniform priors for cell types to avoid sample size bias. Specificity is defined by the posterior probability that a cell is of a certain cell type given it is expressing a particular gene; the detection level is defined by the relative fraction of cells expressing a particular gene. FIG. 3G shows expression profiles of 13 microglia signature genes meeting the specificity threshold of 0.6 and an effect size threshold of 2.5, and perivascular gene markers Mrc1 and Pf4, as well as, T cell markers Cd3g and Ms4a4; the mean expression of the Complement C1q subcomponents a, b, and c is shown.

FIGS. 4A-4E show characterization of activated microglia populations. FIG. 4A shows that unsupervised clustering identified 10 distinct subpopulations spanning a trajectory from homeostatic microglia towards four terminal phenotypes. FIG. 4B shows contingency tables counting agreements (diagonal) and disagreements (off-diagonal) between the expression profile of disease activated microglia population (DAM) described by Keren-Shaul et al. (9) and each cluster in this study; quantifications are based on (log 2) 0.5-fold up- and down-regulated genes relative to the homeostatic population. A similarity score is calculated by subtracting the off-diagonal values from the sum of the diagonal values; P-values are calculated testing the overall agreement between both studies. Increasing gene expression similarities along the trajectory from t1 via t6 to this study's DAM cluster, highlighted in red, can be observed. FIG. 4C shows scoring of cell cycle states. Each cell was predicted to be either in G1, G2/M or S phase using machine learning. One cluster highlighted in yellow, Cyc-M, shows strong enrichment of cycling cells. FIG. 4D shows differential expression analysis revealing one cluster, IFN-R, that shows enrichment of genes related to the interferon pathway (FIG. 4D), and one cluster, MHC-II, enriched in genes encoding members of the MHC class II protein complex (FIG. 4E). The expression of selected marker genes is shown in FIG. 4D, and all MHC class I/II genes as annotated in Gene Ontology (GO) are shown in FIG. 4E. Fisher's exact test with false discovery rate (FDR) correction was used for GO term enrichment analysis.

FIGS. 5A-E show genotype-driven effects on microglia fates. FIG. 5A shows a trajectory tree and visualization of computed linear trajectories from t5 towards each terminal cell type. Pseudotime was inferred by fitting principal curves (black lines) in the lower dimensional manifold. Each datapoint represents a cell colorized by its pseudotemporal location along the trajectory. FIG. 5B-5E show relative fraction of each genotype and its replicates over all pseudotime intervals (upper panel); representation was corrected for different samples sizes. Lower panel shows the distributions of estimated fractions of cells in the 90-100% pseudotime interval using Bootstrapping.

FIGS. 6A-6B show hT2AB treatment effects on the microglia trajectory. FIG. 6A shows estimated relative population sizes per time interval along each trajectory starting from the branching point towards the terminal end type. FIG. 6B shows trajectory-based differential expression analysis of the early and late microglial differentiation stages. P-values were calculated using Wald statistics and corrected for multiple testing via false discovery rate (FDR). The FDR was weighted (wFDR) by the sign of the log fold-change S by FDRS and −log 10 transformed. Negative values denote hT2AB-induced downregulation, positive values indicate upregulation. Using an FDR cutoff of 0.01, transcriptional changes were classified into six categories; two transient with an early up-/downregulation converging to baseline level; four permanent with either early and late up-/downregulation or only late up-/downregulation, respectively.

FIGS. 7A-7B show that sustained acute treatment with mT2AB affects microglial responses to pathology differently. FIG. 7A depicts a schematic diagram of murine mT2AB treatment in TREM2CV-5XFAD or TREM2R47H-5XFAD mice. Twenty-week old mice were injected i.p. with murine mT2AB at 30 mg/kg every 3 days for 10 days. Littermates were administered the same concentration of control mIgG1 antibody. Mice were sacrificed 24 hours after the last antibody injection and brains were harvested for immunohistochemistry and biochemical analysis. FIG. 7B shows quantification of cytokines and chemokines, such as IL-10, CXCL10, and CCL4 in the cortex lysates among different treatment groups. *, P<0.05; ***, P<0.001; ****, P<0.0001 by two-way ANOVA with Sidak's multiple comparisons test. Data are shown as mean±SD in (E). TREM2CV-5XFAD, male, mIgG1, n=8; TREM2CV-5XFAD, male, mT2AB, n=9; TREM2CV-5XFAD, female, mIgG1, n=5; TREM2CV-5XFAD, female, mT2AB, n=6; TREM2R47H-5XFAD, male, mIgG1, n=4; TREM2R47H-5XFAD, male, mT2AB, n=4; TREM2R47H-5XFAD, female, mIgG1, n=4; TREM2R47H-5XFAD, female, mT2AB, n=6.

FIGS. S1A-S1D show single-cell RNA-seq quality control. In FIG. S1A, 24 samples were collected and subjected to individual read alignment, droplet, and cell quality control. FIG. S1B shows integrative data quality assessment revealing technical artifacts in the data, likely driven by low cellular transcriptome coverage. Cells are colorized by the cell quality (CQ) score derived from a principal component analysis of selected quality metrics. Groups enriched in cells with low CQ scores are indicated. FIG. S1C shows determination of the cut-off for cell filtering using the inverse empirical cumulative CQ score distribution function. The determined threshold of −0.1 retained 8.6% of cells within the low CQ enriched group and 88.9% of all other cells. FIG. S1D shows distribution of common quality metrics for each cell per sample. Samples are characterized by human TREM2 variant, sex, and measured hT2AB brain exposure. Shown is also the ratio between the raw expression of the endogenous Trem2 locus and the human TREM2 transgenic locus in all collected cells.

FIG. S2 shows GO Term Enrichment of IFN-R microglia marker genes. IFN-R marker genes meeting a specificity threshold of 0.75 and an effect size of 1.5 were subjected to a Gene Ontology term enrichment analysis. Shown are biological process terms with a false-discovery rate (FDR) corrected Fisher's exact test P-value <0.05. Terms are grouped and colored by their ontological relation, square sizes correspond to the number of IFN-R marker genes found in this category, and color depth is scaled by FDR.

FIGS. S3A-S3B show sample harmonization. FIG. S3A shows annotated microglia cells from control hIgG1-treated female and male TREM2cv-5XFAD mice used as reference to classify cells from the remaining 5 conditions (query Q1-Q5). First, each query set was co-embedded with each reference set using diffusion maps and subsequently corrected for batch effects using mutual nearest neighbor correction. Then cell types were projected using an Extreme Gradient Boosting classifier trained on the diffusion components of the query cells. FIG. S3B shows model accuracy assessments. Training accuracy on the reference or query dataset, as well as, prediction accuracy of projecting cell types on the reference dataset using a classifier trained on the query dataset.

FIGS. S4A-S4E show mouse sex-associated differences in microglia fates. Shown are the distributions of the estimated fractions of cells in the terminal 90-100% pseudotime interval using Bootstrapping (upper panel) and the fitted expression dynamics for the top 10 marker genes (lower panel) for the DAM (FIG. S4A), Cyc-M (FIG. S4B), IFN-R (FIG. S4C), and MHC-II (FIG. S4D) clusters. FIG. S4E shows quantification of soluble and insoluble Ar31_40 and A131 42 in the hippocampus lysates of control hIgG1-treated 5XFAD mice. *, P<0.05; **, P<0.01; ***, P<0.001 by two-way ANOVA with Sidak's multiple comparisons test. Data are shown as mean±SD; left hand bars show data for male mice, right hand bars show data for female mice.

FIGS. S5A-C show sustained acute treatment of murine mT2AB does not change Aβ load. FIG. S5A shows quantification of soluble and insoluble Ar31_40 and Ar31_42 in the cortex lysates among different treatment groups. FIG. S5B show representative confocal images from TREM2cv-5XFAD mice treated with mouse mT2AB stained with 6E10 (white), methoxy-X04 (blue), and Aβ42 (red), displaying the distribution of Aβ in the cortex. Bar=100 Rm. FIG. S5C shows fold changes of 6E10+, methoxy-X04+ or Aβ42+ area coverage in the cortex between murine mT2AB and control mIgG1 treated groups. Data are shown as mean±SEM. TREM2cv-5XFAD, male, mIgG1, n=8; TREM2cv-5XFAD, male, mT2AB, n=9; TREM2cv-5XFAD, female, mIgG1, n=5; TREM2cv-5XFAD, female, mT2AB, n=6; TREM2R47H-5XFAD, male, mIgG1, n=4; TREM2R47H-5XFAD, male, mT2AB, n=4; TREM2R47H-5XFAD, female, mIgG1, n=4; TREM2R47H-5XFAD, female, mT2AB, n=6.′, P<0.001; ****, P<0.0001 by two-way ANOVA with Sidak's multiple comparisons test. Data are shown as mean±SEM.

### 6. DETAILED DESCRIPTION

### 6.1. Definitions

Unless defined otherwise, all technical and scientific terms used herein have the same meaning as commonly understood by one of ordinary skill in the art. Accordingly, the following terms are intended to have the following meanings.

“Agonist” or an “activating” agent, such as a compound or antibody, is an agent that induces (e.g., increases) one or more activities or functions of the target (e.g., TREM2) of the agent after the agent binds the target.

“Antagonist” or a “blocking” agent, such as a compound or antibody, is an agent that reduces or eliminates (e.g., decreases) binding of the target to one or more ligands after the agent binds the target, and/or that reduces or eliminates (e.g., decreases) one or more activities or functions of the target after the agent binds the target. In some embodiments, antagonist agent, or blocking agent substantially or completely inhibits target binding to one or more of its ligand and/or one or more activities or functions of the target.

“Antibody” is used in the broadest sense and refers to an immunoglobulin or fragment thereof, and encompasses any such polypeptide comprising an antigen-binding fragment or region of an antibody. The recognized immunoglobulin genes include the kappa, lambda, alpha, gamma, delta, epsilon and mu constant region genes, as well as myriad immunoglobulin variable region genes. Light chains are generally classified as either kappa or lambda. Heavy chains are classified as gamma, mu, alpha, delta, or epsilon, which in turn define the immunoglobulin classes, IgG, IgM, IgA, IgD and IgE, respectively. Immunoglobulin classes may also be further classified into subclasses, including IgG subclasses IgG₁, IgG₂, IgG₃, and IgG₄; and IgA subclasses IgA₁ and IgA₂. The term includes, but is not limited to, polyclonal, monoclonal, monospecific, multispecific (e.g., bispecific antibodies), natural, humanized, human, chimeric, synthetic, recombinant, hybrid, mutated, grafted, antibody fragments (e.g., a portion of a full-length antibody, generally the antigen binding or variable region thereof, e.g., Fab, Fab′, F(ab′)2, and Fv fragments), and in vitro generated antibodies so long as they exhibit the desired biological activity. The term also includes single chain antibodies, e.g., single chain Fv (sFv or scFv) antibodies, in which a variable heavy and a variable light chain are joined together (directly or through a peptide linker) to form a continuous polypeptide.

The antibodies described herein are, in many embodiments, described by way of their respective polypeptide sequences using single letter amino acid notation. Unless indicated otherwise, polypeptide sequences are provided in N→C orientation.

“Isolated” refers to a change from a natural state, that is, changed and/or removed from its original environment. For example, a polynucleotide or polypeptide (e.g., an antibody) is isolated when it is separated from material with which it is naturally associated in the natural environment. Thus, an “isolated antibody” is one which has been separated and/or recovered from a component of its natural environment.

“Purified antibody” refers to an antibody preparation in which the antibody is at least 80% or greater, at least 85% or greater, at least 90% or greater, at least 95% or greater by weight as compared to other contaminants (e.g., other proteins) in the preparation, such as by determination using SDS-polyacrylamide gel electrophoresis (PAGE) or capillary electrophoresis- (CE) SDS under reducing or non-reducing conditions.

“Extracellular domain” and “ectodomain” are used interchangeably when used in reference to a membrane bound protein and refer to the portion of the protein that is exposed on the extracellular side of a lipid membrane of a cell.

“Binds specifically” in the context of any binding agent, e.g., an antibody, refers to a binding agent that binds specifically to an antigen or epitope, such as with a high affinity, and does not significantly bind other unrelated antigens or epitopes.

“Functional” refers to a form of a molecule which possesses either the native biological activity of the naturally existing molecule of its type, or any specific desired activity, for example as judged by its ability to bind to ligand molecules. Examples of “functional” polypeptides include an antibody binding specifically to an antigen through its antigen-binding region.

“Antigen” refers to a substance, such as, without limitation, a particular peptide, protein, nucleic acid, or carbohydrate which can bind to a specific antibody.

“Epitope” or “antigenic determinant” refers to that portion of an antigen capable of being recognized and specifically bound by a particular antibody. When the antigen is a polypeptide, epitopes can be formed from contiguous amino acids and/or noncontiguous amino acids juxtaposed by tertiary folding of a protein. Linear epitope is an epitope formed from contiguous amino acids on the linear sequence of amino acids. A linear epitope may be retained upon protein denaturing. Conformational or structural epitope is an epitope composed of amino acid residues that are not contiguous and thus comprised of separated parts of the linear sequence of amino acids that are brought into proximity to one another by folding of the molecule, such as through secondary, tertiary, and/or quaternary structures. A conformational or structural epitope may be lost upon protein denaturation. In some embodiments, an epitope can comprise at least 3, and more usually, at least 5 or 8-10 amino acids in a unique spatial conformation. Thus, an epitope as used herein encompasses a defined epitope in which an antibody binds only portions of the defined epitope. There are many methods known in the art for mapping and characterizing the location of epitopes on proteins, including solving the crystal structure of an antibody-antigen complex, competition assays, gene fragment expression assays, mutation assays, and synthetic peptide-based assays, as described, for example, in Using Antibodies: A Laboratory Manual, Chapter 11, Harlow and Lane, eds., Cold Spring Harbor Laboratory Press, Cold Spring Harbor, New York (1999).

“Protein,” “polypeptide,” or “peptide” denotes a polymer of at least two amino acids covalently linked by an amide bond, regardless of length or post-translational modification (e.g., glycosylation, phosphorylation, lipidation, myristoylation, ubiquitination, etc.). Included within this definition are D- and L-amino acids, and mixtures of D- and L-amino acids. Unless specified otherwise, the amino acid sequences of a protein, polypeptide, or peptide are displayed herein in the conventional N-terminal to C-terminal orientation.

“Polynucleotide” and “nucleic acid” are used interchangeably herein and refer to two or more nucleosides that are covalently linked together. The polynucleotide may be wholly comprised of ribonucleosides (i.e., an RNA), wholly comprised of 2′ deoxyribonucleotides (i.e., a DNA) or mixtures of ribo- and 2′ deoxyribonucleosides. The nucleosides will typically be linked together by sugar-phosphate linkages (sugar-phosphate backbone), but the polynucleotides may include one or more non-standard linkages. Non-limiting example of such non-standard linkages include phosphoramidates, phosphorothioates, and amides (see, e.g., Eckstein, F., Oligonucleotides and Analogues: A Practical Approach, Oxford University Press (1992)).

“Operably linked” or “operably associated” refers to a situation in which two or more polynucleotide sequences are positioned to permit their ordinary functionality. For example, a promoter is operably linked to a coding sequence if it is capable of controlling the expression of the sequence. Other control sequences, such as enhancers, ribosome binding or entry sites, termination signals, polyadenylation sequences, and signal sequences are also operably linked to permit their proper function in transcription or translation.

“Amino acid position” and “amino acid residue” are used interchangeably to refer to the position of an amino acid in a polypeptide chain. In some embodiments, the amino acid residue can be represented as “XN”, where X represents the amino acid and the N represents its position in the polypeptide chain. Where two or more variations, e.g., polymorphisms, occur at the same amino acid position, the variations can be represented with a “/” separating the variations. A substitution of one amino acid residue with another amino acid residue at a specified residue position can be represented by XNY, where X represents the original amino acid, N represents the position in the polypeptide chain, and Y represents the replacement or substitute amino acid. When the terms are used to describe a polypeptide or peptide portion in reference to a larger polypeptide or protein, the first number referenced describes the position where the polypeptide or peptide begins (i.e., amino end) and the second referenced number describes where the polypeptide or peptide ends (i.e., carboxy end).

“Polyclonal” antibody refers to a composition of different antibody molecules which is capable of binding to or reacting with several different specific antigenic determinants on the same or on different antigens. A polyclonal antibody can also be considered to be a “cocktail of monoclonal antibodies.” The polyclonal antibodies may be of any origin, e.g., chimeric, humanized, or fully human.

“Monoclonal antibody” refers to an antibody obtained from a population of substantially homogeneous antibodies, i.e., the individual antibodies comprising the population are identical except for possible naturally occurring mutations that may be present in minor amounts. Each monoclonal antibody is directed against a single determinant on the antigen. In some embodiments, monoclonal antibodies to be used in accordance with the present disclosure can be made by the hybridoma method described by Kohler et al., 1975, Nature 256:495-7, or by recombinant DNA methods. The monoclonal antibodies can also be isolated, e.g., from phage antibody libraries.

“Chimeric antibody” refers to an antibody made up of components from at least two different sources. A chimeric antibody can comprise a portion of an antibody derived from a first species fused to another molecule, e.g., a portion of an antibody derived from a second species. In some embodiments, a chimeric antibody comprises a portion of an antibody derived from a non-human animal, e.g., mouse or rat, fused to a portion of an antibody derived from a human. In some embodiments, a chimeric antibody comprises all or a portion of a variable region of an antibody derived from a non-human animal fused to a constant region of an antibody derived from a human.

“Humanized antibody” refers to an antibody that comprises a donor antibody binding specificity, e.g., the CDR regions of a donor antibody, such as a mouse monoclonal antibody, grafted onto human framework sequences. A “humanized antibody” typically binds to the same epitope as the donor antibody.

“Fully human antibody” or “human antibody” refers to an antibody that comprises human immunoglobulin protein sequences only. A fully human antibody may contain murine carbohydrate chains if produced in a non-human cell, e.g., a mouse, in a mouse cell, or in a hybridoma derived from a mouse cell.

“Full-length antibody,” “intact antibody” or “whole antibody” are used interchangeably to refer to an antibody, such as an anti-TREM2 antibody of the present disclosure, in its substantially intact form, as opposed to an antibody fragment. Specifically whole antibodies include those with heavy and light chains including an Fc region. The constant domains may be native sequence constant domains {e.g., human native sequence constant domains) or amino acid sequence variants thereof. In some cases, the intact antibody may have one or more effector functions.

“Antibody fragment” or “antigen-binding moiety” refers to a portion of a full length antibody, generally the antigen binding or variable domain thereof. Examples of antibody fragments include Fab, Fab′, F(ab′)2, and Fv fragments; diabodies; linear antibodies; single-chain antibodies; and multispecific antibodies formed from antibody fragments that bind two or more different antigens. Several examples of antibody fragments containing increased binding stoichiometries or variable valencies (2, 3 or 4) include triabodies, trivalent antibodies and trimerbodies, tetrabodies, tandAbs®, di-diabodies and (sc(Fv)2)₂ molecules, and all can be used as binding agents to bind with high affinity and avidity to soluble antigens (see, e.g., Cuesta et al., 2010, Trends Biotech. 28:355-62).

“Single-chain Fv” or “sFv” antibody fragment comprises the VH and VL domains of an antibody, where these domains are present in a single polypeptide chain. Generally, the Fv polypeptide further comprises a polypeptide linker between the VH and VL domains which enables the sFv to form the desired structure for antigen binding. For a review of sFv, see Pluckthun in The Pharmacology of Monoclonal Antibodies, Vol. 113, pp. 269-315, Rosenberg and Moore, eds., Springer-Verlag, New York (1994).

“Diabodies” refers to small antibody fragments with two antigen-binding sites, which comprise a heavy chain variable domain (VH) connected to a light chain variable domain (VL) in the same polypeptide chain (VH-VL). By using a linker that is short to allow pairing between the two domains on the same chain, the domains are forced to pair with the complementary domains of another chain and create two antigen-binding sites.

“Antigen binding domain” or “antigen binding portion” refers to the region or part of the antigen binding molecule that specifically binds to and complementary to part or all of an antigen. In some embodiments, an antigen binding domain may only bind to a particular part of the antigen (e.g., an epitope), particularly where the antigen is large. An antigen binding domain may comprise one or more antibody variable regions, particularly an antibody light chain variable region (VL) and an antibody heavy chain variable region (VH), and particularly the complementarity determining regions (CDRs) on each of the VH and VL chains.

“Variable region” and “variable domain” are used interchangeably to refer to the polypeptide region that confers the binding and specificity characteristics of each particular antibody. The variable region in the heavy chain of an antibody is referred to as “VH” while the variable region in the light chain of an antibody is referred to as “VL”. The major variability in sequence is generally localized in three regions of the variable domain, denoted as “hypervariable regions” or “CDRs” in each of the VL region and VH region, and forms the antigen binding site. The more conserved portions of the variable domains are referred to as the framework region FR.

“Complementarity-determining region” and “CDR” are used interchangeably to refer to noncontiguous antigen binding regions found within the variable region of the heavy and light chain polypeptides of an antibody molecule. In some embodiments, the CDRs are also described as “hypervariable regions” or “HVR”. Generally, naturally occurring antibodies comprise six CDRs, three in the VH (referred to as: CDR H1 or H1; CDR H2 or H2; and CDR H3 or H3) and three in the VL (referred to as: CDR L1 or L1; CDR L2 or L2; and CDR L3 or L3). The CDR domains have been delineated using various approaches, and it is to be understood that CDRs defined by the different approaches are to be encompassed herein. The “Kabat” approach for defining CDRs uses sequence variability and is the most commonly used (Kabat et al., 1991, “Sequences of Proteins of Immunological Interest, 5^{th} Ed.” NIH 1:688-96). “Chothia” uses the location of structural loops (Chothia and Lesk, 1987, J Mol Biol. 196:901-17). CDRs defined by “AbM” are a compromise between the Kabat and Chothia approach, and can be delineated using Oxford Molecular AbM antibody modeling software (see, Martin et al., 1989, Proc. Natl Acad Sci USA. 86:9268; see also, world wide web www.bioinf-org.uk/abs). The “Contact” CDR delineations are based on analysis of known antibody-antigen crystal structures (see, e.g., MacCallum et al., 1996, J. Mol. Biol. 262, 732-45). The CDRs delineated by these methods typically include overlapping or subsets of amino acid residues when compared to each other.

It is to be understood that the exact residue numbers which encompass a particular CDR will vary depending on the sequence and size of the CDR, and those skilled in the art can routinely determine which residues comprise a particular CDR given the amino acid sequence of the variable region of an antibody.

Kabat, supra, also defined a numbering system for variable domain sequences that is applicable to any antibody. The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU or, Kabat numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. References to residue numbers in the variable domain of antibodies means residue numbering by the Kabat numbering system. References to residue numbers in the constant domain of antibodies means residue numbering by the EU or, Kabat numbering system {e.g., see United States Patent Publication No. 2010-280227). One of skill in the art can assign this system of “Kabat numbering” to any variable domain sequence.

Accordingly, unless otherwise specified, references to the number of specific amino acid residues in an antibody or antigen binding fragment are according to the Kabat numbering system.

“Framework region” or “FR region” refers to amino acid residues that are part of the variable region but are not part of the CDRs (e.g., using the Kabat, Chothia or AbM definition). The variable region of an antibody generally contains four FR regions: FR1, FR2, FR3 and FR4. Accordingly, the FR regions in a VL region appear in the following sequence: FR_{L}1-CDR L1-FR_{L}2-CDR L2-FR_{L}3-CDR L3-FR_{L}4, while the FR regions in a VH region appear in the following sequence: FR1_{H}-CDR H1-FR_{H}2-CDR H2-FR_{H}3-CDR H3-FR_{H}4.

“Constant region” or “constant domain” refers to a region of an immunoglobulin light chain or heavy chain that is distinct from the variable region. The constant domain of the heavy chain generally comprises at least one of: a CH1 domain, a Hinge (e.g., upper, middle, and/or lower hinge region), a CH2 domain, and a CH3 domain. In some embodiments, the antibody can have additional constant domains CH4 and/or CH5. In some embodiments, an antibody described herein comprises a polypeptide containing a CH1 domain; a polypeptide comprising a CH1 domain, at least a portion of a Hinge domain, and a CH2 domain; a polypeptide comprising a CH1 domain and a CH3 domain; a polypeptide comprising a CH1 domain, at least a portion of a Hinge domain, and a CH3 domain, or a polypeptide comprising a CH1 domain, at least a portion of a Hinge domain, a CH2 domain, and a CH3 domain. In some embodiments, the antibody comprises a polypeptide which includes a CH3 domain. The constant domain of a light chain is referred to a CL, and in some embodiments, can be a kappa or lambda constant region. However, it will be understood by one of ordinary skill in the art that these constant domains (e.g., the heavy chain or light chain) may be modified such that they vary in amino acid sequence from the naturally occurring immunoglobulin molecule.

“Fc region” or “Fc portion” refers to the C terminal region of an immunoglobulin heavy chain. The Fc region can be a native-sequence Fc region or a non-naturally occurring variant Fc region. Generally, the Fc region of an immunoglobulin comprises constant domains CH2 and CH3. Although the boundaries of the Fc region can vary, in some embodiments, the human IgG heavy chain Fc region can be defined to extend from an amino acid residue at position C226 or from P230 to the carboxy terminus thereof. In some embodiments, the “CH2 domain” of a human IgG Fc region, also denoted as “Cy2”, generally extends from about amino acid residue 231 to about amino acid residue 340. In some embodiments, N-linked carbohydrate chains can be interposed between the two CH2 domains of an intact native IgG molecule. In some embodiments, the CH3 domain” of a human IgG Fc region comprises residues C-terminal to the CH2 domain, e.g., from about amino acid residue 341 to about amino acid residue 447 of the Fc region. A “functional Fc region” possesses an “effector function” of a native sequence Fc region. Exemplary Fc “effector functions” include, among others, C1q binding; complement dependent cytotoxicity (CDC); Fc receptor binding; antibody dependent cell-mediated cytotoxicity (ADCC); phagocytosis; down regulation of cell-surface receptors (e.g., LT receptor); etc. Such effector functions generally require the Fc region to be combined with a binding domain (e.g., an antibody variable domain) and can be assessed using various assays known in the art.

“Native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

“Variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

“Affinity-matured” antibody, such as an affinity matured anti-TREM2 antibody of the present disclosure, is one with one or more alterations in one or more HVRs thereof that result in an improvement in the affinity of the antibody for antigen, compared to a parent antibody that does not possess those alteration(s). In one embodiment, an affinity-matured antibody has nanomolar or even picomolar affinities for the target antigen. Affinity-matured antibodies are produced by procedures known in the art. For example, Marks et al., Bio/Technology, 1992, 10:779-783 describes affinity maturation by VH- and VL-domain shuffling. Random mutagenesis of HVR and/or framework residues is described by, for example: Barbas et al., Proc Nat. Acad. Sci. USA., 1994, 91:3809-3813; Schier et al. Gene, 1995, 169: 147-155; Yelton et al., Immunol., 1995, 155: 1994-2004; Jackson et al., Immunol., 1995, 154(7):3310-9; and Hawkins et al, J. Mol. Biol., 1992, 226:889-896.

“Binding affinity” refers to strength of the sum total of noncovalent interactions between a ligand and its binding partner. In some embodiments, binding affinity is the intrinsic affinity reflecting a one-to-one interaction between the ligand and binding partner. The affinity is generally expressed in terms of equilibrium association (K_{A}) or dissociation constant (K_{D}), which are in turn reciprocal ratios of dissociation (k_{off}) and association rate constants (kₒₙ).

“Percent (%) sequence identity” and “percentage sequence homology” are used interchangeably herein to refer to comparisons among polynucleotides or polypeptides, and are determined by comparing two optimally aligned sequences over a comparison window, wherein the portion of the polynucleotide or polypeptide sequence in the comparison window may comprise gaps as compared to the reference sequence for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman, 1981, Adv Appl Math. 2:482, by the homology alignment algorithm of Needleman and Wunsch, 1970, J Mol Biol. 48:443, by the search for similarity method of Pearson and Lipman, 1988, Proc Natl Acad Sci USA. 85:2444-8, and particularly by computerized implementations of these algorithms (e.g., BLAST, ALIGN, GAP, BESTFIT, FASTA, and TFASTA; see, e.g., Mount, D. W., Bioinformatics: Sequence and Genome Analysis, 2^{nd} Ed., Cold Spring Harbor Laboratory Press, Cold Spring Harbor, New York (2013))

Examples of algorithms that are suitable for determining percent sequence identity and sequence similarity are the BLAST and BLAST 2.0, FASTDB, or ALIGN algorithms, which are publically available (e.g., NCBI: National Center for Biotechnology Information). Those skilled in the art can determine appropriate parameters for aligning sequences. For example, the BLASTN program (for nucleotide sequences) can use as defaults a word length (W) of 11, an expectation (E) of 10, M=5, N=−4, and a comparison of both strands. Comparison of amino acid sequences using BLASTP can use as defaults a word length (W) of 3, an expectation (E) of 10, and the BLOSUM62 scoring matrix (see Henikoff and Henikoff, 1989, Proc Natl Acad Sci USA. 89:10915-9).

“Amino acid substitution” refers to the replacement of one amino acid in a polypeptide with another amino acid. A “conservative amino acid substitution” refers to the interchangeability of residues having similar side chains, and thus typically involves substitution of the amino acid in the polypeptide with amino acids within the same or similar defined class of amino acids. By way of example and not limitation, an amino acid with an aliphatic side chain may be substituted with another aliphatic amino acid, e.g., alanine, valine, leucine, isoleucine, and methionine; an amino acid with hydroxyl side chain is substituted with another amino acid with a hydroxyl side chain, e.g., serine and threonine; an amino acid having aromatic side chains is substituted with another amino acid having an aromatic side chain, e.g., phenylalanine, tyrosine, tryptophan, and histidine; an amino acid with a basic side chain is substituted with another amino acid with a basic side chain, e.g., lysine, arginine, and histidine; an amino acid with an acidic side chain is substituted with another amino acid with an acidic side chain, e.g., aspartic acid or glutamic acid; and a hydrophobic or hydrophilic amino acid is replaced with another hydrophobic or hydrophilic amino acid, respectively.

“Amino acid insertion” refers to the incorporation of at least one amino acid into a predetermined amino acid sequence. An insertion can be the insertion of one or two amino acid residues; however, larger insertions of about three to about five, or up to about ten or more amino acid residues are contemplated herein.

“Amino acid deletion” refers to the removal of one or more amino acid residues from a predetermined amino acid sequence. A deletion can be the removal of one or two amino acid residues; however, larger deletions of about three to about five, or up to about ten or more amino acid residues are contemplated herein.

“Subject” refers to a mammal, including, but not limited to humans, non-human primates, and non-primates, such as goats, horses, and cows. In some embodiments, the terms “subject” and “patient” are used interchangeably herein in reference to a human subject.

“Therapeutically effective dose” or “therapeutically effective amount” or “effective dose”” refers to that quantity of a compound, including a biologic compound, or pharmaceutical composition that is sufficient to result in a desired activity upon administration to a mammal in need thereof. As used herein, with respect to the pharmaceutical compositions comprising an antibody, the term “therapeutically effective amount/dose” refers to the amount/dose of the antibody or pharmaceutical composition thereof that is sufficient to produce an effective response upon administration to a mammal.

“Pharmaceutically acceptable” refers to compounds or compositions which are generally safe, non-toxic and neither biologically nor otherwise undesirable, and includes a compound or composition that is acceptable for human pharmaceutical and veterinary use. The compound or composition may be approved or approvable by a regulatory agency or listed in the U.S. Pharmacopeia or other generally recognized pharmacopeia for use in animals, including humans.

“Pharmaceutically acceptable excipient, carrier or adjuvant” refers to an excipient, carrier or adjuvant that can be administered to a subject, together with at least one therapeutic agent (e.g., an antibody of the present disclosure), and which does not destroy the pharmacological activity thereof and is generally safe, nontoxic and neither biologically nor otherwise undesirable when administered in doses sufficient to deliver a therapeutic amount of the agent.

The term “treatment” is used interchangeably herein with the term “therapeutic method” and refers to both 1) therapeutic treatments or measures that cure, slow down, lessen symptoms of, and/or halt progression of a diagnosed pathologic conditions, disease or disorder, and 2) and prophylactic/preventative measures. Those in need of treatment may include individuals already having a particular medical disease or disorder as well as those who may ultimately acquire the disorder (i.e., those at risk or needing preventive measures).

The term “subject” or “patient” as used herein refers to any individual to which the subject methods are performed. Generally, the subject is human, although as will be appreciated by those in the art, the subject may be any animal.

In some embodiments, compounds of the present invention are able to cross the blood-brain barrier (BBB). The term “blood-brain barrier” or “BBB”, as used herein, refers to the BBB proper as well as to the blood-spinal barrier. The blood-brain barrier, which consists of the endothelium of the brain vessels, the basal membrane and neuroglial cells, acts to limit penetration of substances into the brain. In some embodiments, the brain/plasma ratio of total drug is at least approximately 0.01 after administration (e.g. oral or intravenous administration) to a patient. In some embodiments, the brain/plasma ratio of total drug is at least approximately 0.03. In some embodiments, the brain/plasma ratio of total drug is at least approximately 0.06. In some embodiments, the brain/plasma ratio of total drug is at least approximately 0.1. In some embodiments, the brain/plasma ratio of total drug is at least approximately 0.2.

The term “homologue,” especially “TREM homologue” as used herein refers to any member of a series of peptides or nucleic acid molecules having a common biological activity, including antigenicity/immunogenicity and inflammation regulatory activity, and/or structural domain and having sufficient amino acid or nucleotide sequence identity as defined herein. TREM homologues can be from either the same or different species of animals.

The term “variant” as used herein refers either to a naturally occurring allelic variation of a given peptide or a recombinantly prepared variation of a given peptide or protein in which one or more amino acid residues have been modified by amino acid substitution, addition, or deletion.

The term “derivative” as used herein refers to a variation of given peptide or protein that are otherwise modified, i.e., by covalent attachment of any type of molecule, preferably having bioactivity, to the peptide or protein, including non-naturally occurring amino acids.

### 6.2. General Description of Certain Embodiments of the Invention

Diagnosis, prognosis, and treatment of Alzheimer's disease in a patient is greatly aided by the identification of changes in levels and types of cells in the plaque microenvironment, expression patterns for sets of genes of cells associated with the plaque microenvironment, cytokine expression levels, immunological response factors, or other changes in the plaque microenvironment, referred to herein generally as “biomarkers” or more specifically in relation to gene expression patterns as “gene signatures,” “gene expression biomarkers,” or “molecular signatures,” or in relation to protein expression patterns as “protein signatures,” “protein expression biomarkers,” or “proteome signatures,” or in relation to cell-type composition patterns as “cell signatures” (i.e., microglial cell signatures), which are characteristic of Alzheimer's disease. Such biomarkers may be associated with clinical outcomes. If such an association is predictive of a clinical response, the biomarker is advantageously used in methods of selecting or stratifying patients as more (or less, as the case may be) likely to benefit from a treatment regimen, such as one of those disclosed herein.

Biological samples from a patient with biomarker profiles that are predictive of a positive response to treatment are referred to herein as “biomarker positive” or “biomarker high.” Conversely, biological samples from a patient with biomarker profiles that are not predictive of a positive response are referred to herein as “biomarker negative” or “biomarker low.” Alternative terms can be used depending upon the biomarker, but a higher amount, or “biomarker high” usually can be described using alternative terminology, such as “biomarker positive” or “biomarker +” while a lower amount of a biomarker or “biomarker low” usually can be described using alternative terminology, such as “biomarker negative” or “biomarker −.”

In some embodiments, a biomarker used in the present invention is a biomarker panel, such as a gene expression panel. In other embodiments, a biomarker panel is a cytokine panel. In other embodiments a biomarker panel is a characterization of cell types present in the plaque microenvironment.

Such a “panel,” as used herein, refers to a group of specific biomarkers, e.g., specific genes or specific cell type populations in the plaque microenvironment, that respond to a particular stimulus (e.g., treatment of the patient with a TREM2 agonist), in a way that tends to predict the likelihood of a particular clinical outcome. Individual biomarkers, e.g., expression of a gene or prevalence of a particular cell type, in a panel need not each respond in the same way. Some may be up-regulated and some may be down-regulated; accordingly, the overall response of the panel is generally the most useful in predicting the likelihood of a clinical response.

In some embodiments, a biomarker used in the present invention is a gene signature. In other embodiments, a biomarker is a cytokine signature. In other embodiments a biomarker panel is a cell type signature of cells in the plaque microenvironment. Similar to a panel, a “signature” as used herein refers to a group of biomarkers, such as specific genes or specific cell type populations present in the plaque microenvironment, that respond to a stimulus to provide a fingerprint (distinctive pattern) of biomarker response to treatment.

Furthermore, while Alzheimer's disease patient derived biomarkers are an important tool in improving the diagnosis, prognosis, and treatment of Alzheimer's disease, the invasiveness of collecting biological samples may increase the risk of serious complications, including anesthetic catastrophes, hemorrhage, infection, seizures and death (Warren et al, Brain, 2005). Both the surgical removal of brain tissue (biopsy) and the aspiration of cells from plaque sites (fine needle aspiration cytology) have the potential to expose abnormal cells to the cranial cavity. The reduced invasiveness of collecting serum samples for biomarker analysis relative to biopsy allows for more continuous monitoring of patient response to treatment. Consequently, minimally invasive diagnostic tools and methods that avoid disrupting cranial integrity or causing inflammation, such as “serum biomarkers,” present opportunities to improve patient care while mitigating risks associated with current treatment regimens. Serum biomarkers include biomarkers that may be obtained by a bodily fluid sample obtained remote from plaque sites (e.g., venous blood and lymph fluid). Examples of serum biomarkers include, for example, circulating cytokines and growth factors, as well as phenotypic and genotypic markers in circulating immune cells.

### 6.3. Disease-Related Biomarkers

It has been surprisingly found that levels of activation of microglia towards disease activated (DAM), interferon-responsive (IFN-R), cycling (Cyc-M), and MHC-II RNA expressing (MHC II) microglia cell type trajectories may be used as a biomarker in a method described herein, such as a method of treating Alzheimer's disease in a patient, diagnosing Alzheimer's disease in a patient, or predicting patient response to treatment of Alzheimer's disease. In some embodiments of the invention, the biomarker comprises a status of microglial cell state transition. In some embodiments the microglial cell state transition is from a homeostatic state. In some embodiments, the microglial cell state transition is towards a DAM, IFN-R, Cyc-M, or MHC-II microglial cell type trajectory. In some embodiments, determination of the microglial cell state is by cell sorting.

A gene from an expression profile that is characteristic of a microglial cell is suitable as a biomarker in a method described herein. In some embodiments, a biomarker is a gene selected from C1QA/B/C, CD81, HEXB, IL1B, LGMN, OLFML3, P2RY12, SPARC, TMEM119, MRC1, PF4, CD3G, or MS4A4B. In some embodiments, a biomarker is a protein or variant thereof encoded by a gene selected from C1QA/B/C, CD81, HEXB, IL1B, LGMN, OLFML3, P2RY12, SPARC, TMEM119, MRC1, PF4, CD3G, or MS4A4B.

Any gene from an expression profile characteristic of a particular microglial cell state trajectory (e.g., towards a DAM, Cyc-M, IFN-R, or MHC-II microglial cell type) is suitable as a biomarker in a method described herein. In some embodiments, a biomarker is a gene from an expression profile characteristic of a DAM microglia trajectory. In some embodiments, a biomarker is a gene selected from FTL1, MLF, CD63, LPL, CTSB, CST7, APOE, CCL4, CD9, or CCL3. In some embodiments, a biomarker is a protein or variant thereof encoded by a gene selected from Ftl1, MLF, CD63, LPL, CTSB, CST7, APOE, CCL4, CD9, or CCL3. In some embodiments, a biomarker is a gene from an expression profile characteristic of a Cyc-M microglia trajectory. In some embodiments, a biomarker is a gene selected from H2AFZ, HMGB2, TUBA1B, HMGN2, H2AFV, IFI2712A, TUBB5, BIRC5, STMN1, or CCNB2. In some embodiments, a biomarker is a protein or variant thereof encoded by a gene selected from H2AFZ, HMGB2, TUBA1B, HMGN2, H2AFV, IFI2712A, TUBB5, BIRC5, STMN1, or CCNB2. In some embodiments, a biomarker is a gene from an expression profile characteristic of a IFN-R microglia trajectory. In some embodiments, a biomarker is a gene selected from CCL12, IFITM3, ISG15, IFIT3, BST2, OASL2, LGALS3BP, RTP4, IFI204, or IRF7. In some embodiments, a biomarker is a protein or variant thereof encoded by a gene selected from CCL12, IFITM3, ISG15, IFIT3, BST2, OASL2, LGALS3BP, RTP4, IFI204, or IRF7. In some embodiments, a biomarker is a gene from an expression profile characteristic of a MCH II microglia trajectory. In some embodiments, a biomarker is a gene selected from H2-K1, H2-Q7, H2-EB1, CD74, H2-AA, H2-D1, H2-AB1, H2-DMA, H2-T23, or LY6E. In some embodiments, a biomarker is a protein or variant thereof encoded by a gene selected from H2-K1, H2-Q7, H2-EB1, CD74, H2-AA, H2-D1, H2-AB1, H2-DMA, H2-T23, or LY6E.

In some embodiments, a biomarker is a gene associated with the interferon pathway. In some embodiments, a biomarker is a gene selected from BST2, CCL2, IFI204, IFI2712A, IFIT3, IFITM3, IRF7, ISG15, LGALS3BP, OASL2, RTP4, SLFN2, or USP18. In some embodiments, a biomarker is a protein or variant thereof encoded by a gene selected from BST2, CCL2, IFI204, IFI2712A, IFIT3, IFITM3, IRF7, ISG15, LGALS3BP, OASL2, RTP4, SLFN2, or USP18.

In some embodiments, a biomarker is a gene associated with a MHC class I protein complex. In some embodiments, a biomarker is a gene selected from B2M, H2-D1, H2-K1, H2-Q10, H2-Q4, H2-Q6, H2-Q7, H2-T23, or MR1. In some embodiments, a biomarker is a protein or variant thereof encoded by a gene selected from B2M, H2-D1, H2-K1, H2-Q10, H2-Q4, H2-Q6, H2-Q7, H2-T23, or MR1. In some embodiments, a biomarker is a gene related to the MHC class II protein complex. In some embodiments, a biomarker is a gene selected from CD74, H2-AA, H2-AB1, H2-DMA, H2-DMB1, H2-DMB2, H2-EB1, H2-OA, or H2-OB. In some embodiments, a biomarker is a protein or variant thereof encoded by a gene selected from CD74, H2-AA, H2-AB1, H2-DMA, H2-DMB1, H2-DMB2, H2-EB1, H2-OA, or H2-OB.

It has been found that treatment with a TREM2 agonist increases the levels of CCL4, CCL2, CST7, CXCL2, CXCL10, IL1B, and TMEM119 in an Alzheimer's animal disease model, the levels of which may be used as a biomarker in a method described herein, such as a method of treating Alzheimer's disease in a patient, diagnosing Alzheimer's disease in a patient, or predicting patient response to treatment of Alzheimer's disease. In some embodiments, a biomarker is a gene selected from CCL2, CCL4, CST7, CXCL2, CXCL10, IL1B, or TMEM119. In other embodiments, a biomarker is a protein or variant thereof encoded by a gene selected from CCL2, CCL4, CST7, CXCL2, CXCL10, IL1B, or TMEM119.

It is contemplated that in certain aspects of the invention, one or more biomarkers from the same class or different class of biomarkers (i.e., gene biomarkers and microglial cell state biomarkers) may be used alone, or in any combination therewith each other as a biomarker panel when used in a method described herein. In some embodiments, a biomarker panel comprises one or more biomarkers selected from APOE, B2M, BIRC5, BST2, C1QA/B/C, CCL12, CCL2, CCL3, CCL4, CCNB2, CD3G, CD63, CD74, CD81, CD9, CST7, CTSB, CXCL10, CXCL2, FTL1, H2-AA, H2-AB1, H2AFV, H2AFZ, H2-D1, H2-DMA, H2-DMB1, H2-DMB2, H2-EB1, H2-K1, H2-OA, H2-OB, H2-Q10, H2-Q4, H2-Q6, H2-Q7, H2-T23, HEXB, HMGB2, HMGN2, IFI204, IFI2712A, IFIT3, IFITM3, IRF7, ISG15, LGALS3BP, LGMN, LPL, LY6E, MLF, MR1, MRC1, MS4A4B, OASL2, OLFML3, P2RY12, PF4, RTP4, SLFN2, SPARC, STMN1, TMEM119, TUBA1B, TUBB5, or USP18. In some embodiments, the expression level of one or more of the above biomarkers are increased after administration of a TREM2 agonist. In some embodiments, the expression level of one or more of the above biomarkers are decreased after administration of a TREM2 agonist.

In some embodiments, the biomarker panel comprises one or more biomarkers selected from CCL2, CCL4, CST7, CXCL2, CXCL10, IL1B, or TMEM119. In some embodiments, the expression level of one or more biomarkers selected from CCL2, CCL4, CST7, CXCL2, CXCL10, IL1B, or TMEM119 are increased after administration of a TREM2 agonist. In some embodiments, one, two, three, four, or five of the one or more biomarkers selected from CCL2, CCL4, CST7, CXCL2, CXCL10, IL1B, or TMEM119 are increased after administration of a TREM2 agonist. In some embodiments, the biomarkers CCL2, CCL4, CST7, CXCL2, and CXCL10 are increased after administration of a TREM2 agonist. In certain embodiments, the TREM2 agonist is an anti-human TREM2 antibody.

It has also been found that treatment with a TREM2 agonist modulates the levels of one or more biomarkers selected from those shown below in Table A. In some embodiments, the expression level of one or more biomarkers selected from those in Table A are increased after administration of a TREM2 agonist. In some embodiments, the expression level of one or more biomarkers selected from those in Table A are decreased after administration of a TREM2 agonist. In some embodiments, one or more biomarkers listed in Table A can be measured alongside other biomarkers of the present disclosure in any methods contemplated herein.

TABLE A
Selected Additional Biomarkers

| | | | | | |
|---|---|---|---|---|---|
| ABCD1 | Arg1 | CCL8 | CCL19 | CCL21 | CCR7 |
| CD83 | CD86 | CD206 | CH25H | Chi313 | CNTF |
| CRP | CSF2 | CSF3 | CST7 | FasL | FIZZ1 |
| Fzd1 | GM-CSF | HMGB1 | IGF1 | IFN-a4 | IFN-β |
| IFN-γ | IL-1β | IL-4 | IL-6 | IL-8 | IL-10 |
| IL-11 | IL-12 | IL-12p40 | IL-12P70 | IL-17 | IL-18 |
| IL-20 | IL-22 | IL-27 | IL-33 | LIF | MCP-1 |
| OSM | sTREM2 | sCSF1R | TNF | TNF-α | Ym1 |

In some embodiments, the increase or decrease in the level of a biomarker in a patient is a measurable increase or decrease that correlates with an increased (or decreased, as the case may be) likelihood of therapeutic benefit for the patient, or for a group of patients, or a patient or group of patients yet to be selected. In some embodiments, the increase or decrease is a statistically significant increase or decrease. The term “statistical significance” is well-known in the art and may be determined using methods known in the art, such as those described herein. In some embodiments, statistical significance means, e.g., p<0.1, p<0.05, p<0.04, p<0.03, p<0.02, or p<0.01 relative to baseline.

In some embodiments, the increase or decrease in the level of a biomarker is observed after the patient has completed one cycle of treatment. In some embodiments, the increase or decrease is observed after two or more cycles of treatment, such as three, four, five, six, seven, eight, nine, or 10 or more cycles. The term “cycle of treatment” is well-known in the art and refers to a physician-defined treatment regimen followed by a patient for a period of time such as 1, 2, 3, or 4 weeks, optionally followed by a period of, e.g., 1, 2, 3, or 4 weeks of patient recovery and/or disease progression monitoring, during which, in some cases, a lower dose of therapeutic agent (or no therapeutic agent at all) is administered. In some embodiments, a cycle of treatment refers to administering a TREM2 agonist, such as hT2AB described herein or a pharmaceutically acceptable salt thereof, either as a monotherapy, or in combination with another therapy.

### 6.4. Methods of the Invention

Certain aspects of the present invention provide a molecule that increases activity of TREM2 (i.e., a TREM2 agonist) for use in treating, preventing, or ameliorating the risk of developing conditions associated with TREM2 deficiency in a patient in need thereof. Conditions or disorders associated with TREM2 deficiency or loss of TREM2 function that may be prevented, treated, or ameliorated according to the methods of the invention include, but are not limited to, Nasu-Hakola disease, Alzheimer's disease, frontotemporal dementia, multiple sclerosis, Guillain-Barre syndrome, amyotrophic lateral sclerosis, Parkinson's disease, traumatic brain injury, spinal cord injury, systemic lupus erythematosus, rheumatoid arthritis, prion disease, stroke, osteoporosis, osteopetrosis, and osteosclerosis. While aspects of certain methods of the invention are described below for patients having Alzheimer's disease, the methods of the invention are contemplated to encompass patients having any of the above listed conditions or disorders associated with a TREM2 deficiency or loss of TREM2 function. For example, the methods described below are intended to encompass a patient having Nasu-Hakola disease, frontotemporal dementia, multiple sclerosis, prion disease, or stroke. As such, the methods described herein for treating Alzheimer's disease can also be applied to another disease described herein.

In one aspect, the present invention provides a method of identifying a patient with Alzheimer's disease who will benefit from treatment with a TREM2 agonist, comprising:
(a) obtaining a first biological sample from the patient prior to administration of the TREM2 agonist to the patient;
(b) measuring a level in the first biological sample of one or more biomarkers selected from APOE, B2M, BIRC5, BST2, C1QA/B/C, CCL12, CCL2, CCL3, CCL4, CCNB2, CD3G, CD63, CD74, CD81, CD9, CST7, CTSB, CXCL10, CXCL2, FTL1, H2-AA, H2-AB1, H2AFV, H2AFZ, H2-D1, H2-DMA, H2-DMB1, H2-DMB2, H2-EB1, H2-K1, H2-OA, H2-OB, H2-Q10, H2-Q4, H2-Q6, H2-Q7, H2-T23, HEXB, HMGB2, HMGN2, IFI204, IFI2712A, IFIT3, IFITM3, IL1B, IRF7, ISG15, LGALS3BP, LGMN, LPL, LY6E, MLF, MR1, MRC1, MS4A4B, OASL2, OLFML3, P2RY12, PF4, RTP4, SLFN2, SPARC, STMN1, TMEM119, TUBA1B, TUBB5, or USP18;
(c) administering to the patient an effective amount of a TREM2 agonist;
(d) obtaining a second biological sample from the patient after administration of the TREM2 agonist to the patient; and
(e) measuring a level in the second biological sample of one or more biomarkers selected from APOE, B2M, BIRC5, BST2, C1QA/B/C, CCL12, CCL2, CCL3, CCL4, CCNB2, CD3G, CD63, CD74, CD81, CD9, CST7, CTSB, CXCL10, CXCL2, FTL1, H2-AA, H2-AB1, H2AFV, H2AFZ, H2-D1, H2-DMA, H2-DMB1, H2-DMB2, H2-EB1, H2-K1, H2-OA, H2-OB, H2-Q10, H2-Q4, H2-Q6, H2-Q7, H2-T23, HEXB, HMGB2, HMGN2, IFI204, IFI2712A, IFIT3, IFITM3, IL1B, IRF7, ISG15, LGALS3BP, LGMN, LPL, LY6E, MLF, MR1, MRC1, MS4A4B, OASL2, OLFML3, P2RY12, PF4, RTP4, SLFN2, SPARC, STMN1, TMEM119, TUBA1B, TUBB5, or USP18.

In certain embodiments, the one or more biomarkers are selected from CCL2, CCL4, CST7, CXCL2, CXCL10, IL1B, or TMEM119. In certain embodiments, the TREM2 agonist is an anti-hTREM2 antibody. In some embodiments, steps (b) and (e) further comprise measuring the level of one or more biomarkers selected from those listed in Table A.

In another aspect, the present invention provides a method of identifying a patient with a Alzheimer's disease who is likely to benefit, or has an increased probability of benefitting relative to an otherwise similar patient, from treatment with a TREM2 agonist, comprising:
(a) obtaining a first biological sample from the patient prior to administration of the TREM2 agonist to the patient;
(b) measuring a level in the first biological sample of one or more biomarkers selected APOE, B2M, BIRC5, BST2, C1QA/B/C, CCL12, CCL2, CCL3, CCL4, CCNB2, CD3G, CD63, CD74, CD81, CD9, CST7, CTSB, CXCL10, CXCL2, FTL1, H2-AA, H2-AB1, H2AFV, H2AFZ, H2-D1, H2-DMA, H2-DMB1, H2-DMB2, H2-EB1, H2-K1, H2-OA, H2-OB, H2-Q10, H2-Q4, H2-Q6, H2-Q7, H2-T23, HEXB, HMGB2, HMGN2, IFI204, IFI2712A, IFIT3, IFITM3, IL1B, IRF7, ISG5, LGALS3BP, LGMN, LPL, LY6E, MLF, MR1, MRC1, MS4A4B, OASL2, OLFML3, P2RY12, PF4, RTP4, SLFN2, SPARC, STMN1, TMEM119, TUBA1B, TUBB5, or USP18;
(c) administering to the patient an effective amount of a TREM2 agonist;
(d) obtaining a second biological sample from the patient after administration of the TREM2 agonist to the patient; and
(e) measuring a level in the second biological sample of one or more biomarkers selected from APOE, B2M, BIRC5, BST2, C1QA/B/C, CCL12, CCL2, CCL3, CCL4, CCNB2, CD3G, CD63, CD74, CD81, CD9, CST7, CTSB, CXCL10, CXCL2, FTL1, H2-AA, H2-AB1, H2AFV, H2AFZ, H2-D1, H2-DMA, H2-DMB, H2-DMB2, H2-EB1, H2-K1, H2-OA, H2-OB, H2-Q10, H2-Q4, H2-Q6, H2-Q7, H2-T23, HEXB, HMGB2, HMGN2, IFI204, IFI2712A, IFIT3, IFITM3, IL1B, IRF7, ISG15, LGALS3BP, LGMN, LPL, LY6E, MLF, MR1, MRC1, MS4A4B, OASL2, OLFML3, P2RY12, PF4, RTP4, SLFN2, SPARC, STMN1, TMEM119, TUBA1B, TUBB5, or USP18;
   wherein the Alzheimer's disease response to step (c) is predictive of the likelihood of successful treatment of Alzheimer's disease based on a greater or lesser response of the Alzheimer's disease compared with one or more similar patients and as evaluated using one or more of the biomarkers.

In certain embodiments, the one or more biomarkers are selected from CCL2, CCL4, CST7, CXCL2, CXCL10, IL1B, or TMEM119. In certain embodiments, the TREM2 agonist is an anti-hTREM2 antibody. In some embodiments, steps (b) and (e) further comprise measuring the level of one or more biomarkers selected from those listed in Table A.

In another aspect, the present invention provides a method of assaying a biological sample taken from a patient in vitro or ex vivo to determine if Alzheimer's disease in the patient will respond, or has an increased probability of responding, to treatment with a TREM2 agonist, comprising:
(a) obtaining a first biological sample from the patient prior to administration of the TREM2 agonist to the patient;
(b) measuring a level in the first biological sample of one or more biomarkers selected from APOE, B2M, BIRC5, BST2, C1QA/B/C, CCL12, CCL2, CCL3, CCL4, CCNB2, CD3G, CD63, CD74, CD81, CD9, CST7, CTSB, CXCL10, CXCL2, FTL1, H2-AA, H2-AB1, H2AFV, H2AFZ, H2-D1, H2-DMA, H2-DMB1, H2-DMB2, H2-EB1, H2-K1, H2-OA, H2-OB, H2-Q10, H2-Q4, H2-Q6, H2-Q7, H2-T23, HEXB, HMGB2, HMGN2, IFI204, IFI2712A, IFIT3, IFITM3, IL1B, IRF7, ISG15, LGALS3BP, LGMN, LPL, LY6E, MLF, MR1, MRC1, MS4A4B, OASL2, OLFML3, P2RY12, PF4, RTP4, SLFN2, SPARC, STMN1, TMEM119, TUBA1B, TUBB5, or USP18;
(c) administering to the patient an effective amount of a TREM2 agonist, if the patient with Alzheimer's Disease will respond, or has an increased probability of responding, to treatment with a TREM2 agonist.

In certain embodiments, the one or more biomarkers are selected from CCL2, CCL4, CST7, CXCL2, CXCL10, IL1B, or TMEM119. In certain embodiments, the TREM2 agonist is an anti-hTREM2 antibody. In some embodiments, step (b) further comprises measuring the level of one or more biomarkers selected from those listed in Table A.

In another aspect, the present invention provides a method of treating Alzheimer's disease in a patient who either does not respond to prior Alzheimer's treatment or whose Alzheimer's disease has become refractory after initially responding to prior Alzheimer's treatment, comprising:
(a) obtaining a first biological sample from the patient prior to administration of the TREM2 agonist to the patient;
(b) measuring a level in the first biological sample of one or more biomarkers selected from APOE, B2M, BIRC5, BST2, C1QA/B/C, CCL12, CCL2, CCL3, CCL4, CCNB2, CD3G, CD63, CD74, CD81, CD9, CST7, CTSB, CXCL10, CXCL2, FTL1, H2-AA, H2-AB1, H2AFV, H2AFZ, H2-D1, H2-DMA, H2-DMB1, H2-DMB2, H2-EB1, H2-K1, H2-OA, H2-OB, H2-Q10, H2-Q4, H2-Q6, H2-Q7, H2-T23, HEXB, HMGB2, HMGN2, IFI204, IFI2712A, IFIT3, IFITM3, IL1B, IRF7, ISG15, LGALS3BP, LGMN, LPL, LY6E, MLF, MR1, MRC1, MS4A4B, OASL2, OLFML3, P2RY12, PF4, RTP4, SLFN2, SPARC, STMN1, TMEM119, TUBA1B, TUBB5, or USP18;
(c) administering to the patient an effective amount of a TREM2 agonist, if the patient with Alzheimer's disease will respond, or has an increased probability of responding, to treatment with a TREM2 agonist;
(d) obtaining a second biological sample after administration of the TREM2 agonist to the patient; and
(e) measuring a level in the second biological sample of one or more biomarkers selected from APOE, B2M, BIRC5, BST2, C1QA/B/C, CCL12, CCL2, CCL3, CCL4, CCNB2, CD3G, CD63, CD74, CD81, CD9, CST7, CTSB, CXCL10, CXCL2, FTL1, H2-AA, H2-AB1, H2AFV, H2AFZ, H2-D1, H2-DMA, H2-DMB1, H2-DMB2, H2-EB1, H2-K1, H2-OA, H2-OB, H2-Q10, H2-Q4, H2-Q6, H2-Q7, H2-T23, HEXB, HMGB2, HMGN2, IFI204, IFI2712A, IFIT3, IFITM3, IL1B, IRF7, ISG15, LGALS3BP, LGMN, LPL, LY6E, MLF, MR1, MRC1, MS4A4B, OASL2, OLFML3, P2RY12, PF4, RTP4, SLFN2, SPARC, STMN1, TMEM119, TUBA1B, TUBB5, or USP18;
   wherein the Alzheimer's disease response to step (c) is predictive of the likelihood of successful treatment of Alzheimer's disease based on a greater or lesser response of the Alzheimer's disease compared with one or more similar patients and as evaluated using one or more of the biomarkers.

In certain embodiments, the one or more biomarkers are selected from CCL2, CCL4, CST7, CXCL2, CXCL10, IL1B, or TMEM119. In certain embodiments, the TREM2 agonist is an anti-hTREM2 antibody. In some embodiments, steps (b) and (e) further comprise measuring the level of one or more biomarkers selected from those listed in Table A.

In another aspect, the present invention provides a method of predicting whether Alzheimer's disease will respond to treatment with a second Alzheimer's disease treatment following treatment with a TREM2 agonist, comprising:
(a) obtaining a first biological sample from the patient prior to administration of the TREM2 agonist to the patient;
(b) measuring a level in the first biological sample of one or more biomarkers selected from APOE, B2M, BIRC5, BST2, C1QA/B/C, CCL12, CCL2, CCL3, CCL4, CCNB2, CD3G, CD63, CD74, CD81, CD9, CST7, CTSB, CXCL10, CXCL2, FTL1, H2-AA, H2-AB1, H2AFV, H2AFZ, H2-D1, H2-DMA, H2-DMB1, H2-DMB2, H2-EB1, H2-K1, H2-OA, H2-OB, H2-Q10, H2-Q4, H2-Q6, H2-Q7, H2-T23, HEXB, HMGB2, HMGN2, IFI204, IFI2712A, IFIT3, IFITM3, IL1B, IRF7, ISG15, LGALS3BP, LGMN, LPL, LY6E, MLF, MR1, MRC1, MS4A4B, OASL2, OLFML3, P2RY12, PF4, RTP4, SLFN2, SPARC, STMN1, TMEM119, TUBA1B, TUBB5, or USP18;
(c) administering to the patient an effective amount of a TREM2 agonist;
(d) obtaining a second biological sample after administration of the TREM2 agonist to the patient; and
(e) measuring a level in the second biological sample of one or more biomarkers selected from APOE, B2M, BIRC5, BST2, C1QA/B/C, CCL12, CCL2, CCL3, CCL4, CCNB2, CD3G, CD63, CD74, CD81, CD9, CST7, CTSB, CXCL10, CXCL2, FTL1, H2-AA, H2-AB1, H2AFV, H2AFZ, H2-D1, H2-DMA, H2-DMB1, H2-DMB2, H2-EB1, H2-K1, H2-OA, H2-OB, H2-Q10, H2-Q4, H2-Q6, H2-Q7, H2-T23, HEXB, HMGB2, HMGN2, IFI204, IFI2712A, IFIT3, IFITM3, IL1B, IRF7, ISG15, LGALS3BP, LGMN, LPL, LY6E, MLF, MR1, MRC1, MS4A4B, OASL2, OLFML3, P2RY12, PF4, RTP4, SLFN2, SPARC, STMN1, TMEM119, TUBA1B, TUBB5, or USP18;
   wherein the Alzheimer's disease to step (c) is predictive of the likelihood of successful treatment of the Alzheimer's disease based on a greater or lesser response of the Alzheimer's disease compared with one or more similar patients and as evaluated using one or more of the biomarkers.

In certain embodiments, the one or more biomarkers are selected from CCL2, CCL4, CST7, CXCL2, CXCL10, IL1B, or TMEM119. In certain embodiments, the TREM2 agonist is an anti-hTREM2 antibody. In some embodiments, steps (b) and (e) further comprise measuring the level of one or more biomarkers selected from those listed in Table A.

In some embodiments, treatment with a TREM2 agonist primes the plaque microenvironment such that the plaque becomes more likely to respond to a second Alzheimer's disease therapeutic agent. In some embodiments, the plaque does not respond to monotherapy with an Alzheimer's disease treatment, but becomes primed and responds to the Alzheimer's disease treatment when combined with a TREM2 agonist. In some embodiments, the plaque initially responds to the monotherapy with an Alzheimer's disease treatment, but becomes refractory. In some embodiments, after treatment with a TREM2 agonist, the plaque can be treated effectively with the Alzheimer's disease treatment.

In some embodiments, the above method is useful in the identification of a patient who will benefit from treatment with a TREM2 agonist. Such a patient is characterized in that the level of one or more biomarkers selected from APOE, B2M, BIRC5, BST2, C1QA/B/C, CCL12, CCL2, CCL3, CCL4, CCNB2, CD3G, CD63, CD74, CD81, CD9, CST7, CTSB, CXCL10, CXCL2, FTL1, H2-AA, H2-AB1, H2AFV, H2AFZ, H2-D1, H2-DMA, H2-DMB1, H2-DMB2, H2-EB1, H2-K1, H2-OA, H2-OB, H2-Q10, H2-Q4, H2-Q6, H2-Q7, H2-T23, HEXB, HMGB2, HMGN2, IFI204, IFI2712A, IFIT3, IFITM3, IL1B, IRF7, ISG15, LGALS3BP, LGMN, LPL, LY6E, MLF, MR1, MRC1, MS4A4B, OASL2, OLFML3, P2RY12, PF4, RTP4, SLFN2, SPARC, STMN1, TMEM119, TUBA1B, TUBB5, or USP18 is higher in the second biological sample from the patient than in the first biological sample from the patient. In some embodiments, when the level of one or more biomarkers selected from APOE, B2M, BIRC5, BST2, C1QA/B/C, CCL12, CCL2, CCL3, CCL4, CCNB2, CD3G, CD63, CD74, CD81, CD9, CST7, CTSB, CXCL10, CXCL2, FTL1, H2-AA, H2-AB1, H2AFV, H2AFZ, H2-D1, H2-DMA, H2-DMB1, H2-DMB2, H2-EB1, H2-K1, H2-OA, H2-OB, H2-Q10, H2-Q4, H2-Q6, H2-Q7, H2-T23, HEXB, HMGB2, HMGN2, IFI204, IFI2712A, IFIT3, IFITM3, IL1B, IRF7, ISG15, LGALS3BP, LGMN, LPL, LY6E, MLF, MR1, MRC1, MS4A4B, OASL2, OLFML3, P2RY12, PF4, RTP4, SLFN2, SPARC, STMN1, TMEM119, TUBA1B, TUBB5, or USP18 is higher in the second biological sample from the patient than in the first biological sample from the patient, then the patient is administered one or more additional doses of the TREM2 agonist. This is because such a patient is considered likely to benefit from continued treatment with the TREM2 agonist. In certain embodiments, the one or more biomarkers are selected from CCL2, CCL4, CST7, CXCL2, CXCL10, IL1B, or TMEM119. In certain embodiments, the TREM2 agonist is an anti-hTREM2 antibody. In some embodiments, the patient is characterized in that the level of one or more additional biomarkers selected from those listed in Table A is also higher in the second biological sample than in the first biological sample.

In another aspect, the present invention provides a method of treating Alzheimer's disease with a TREM2 agonist, comprising:
(a) obtaining a first biological sample from the patient prior to administration of the TREM2 agonist to the patient;
(b) measuring a level in the first biological sample of one or more biomarkers selected from APOE, B2M, BIRC5, BST2, C1QA/B/C, CCL12, CCL2, CCL3, CCL4, CCNB2, CD3G, CD63, CD74, CD81, CD9, CST7, CTSB, CXCL10, CXCL2, FTL1, H2-AA, H2-AB1, H2AFV, H2AFZ, H2-D1, H2-DMA, H2-DMB1, H2-DMB2, H2-EB1, H2-K1, H2-OA, H2-OB, H2-Q10, H2-Q4, H2-Q6, H2-Q7, H2-T23, HEXB, HMGB2, HMGN2, IFI204, IFI2712A, IFIT3, IFITM3, IL1B, IRF7, ISG15, LGALS3BP, LGMN, LPL, LY6E, MLF, MR1, MRC1, MS4A4B, OASL2, OLFML3, P2RY12, PF4, RTP4, SLFN2, SPARC, STMN1, TMEM119, TUBA1B, TUBB5, or USP18;
(c) administering to the patient an effective amount of a TREM2 agonist;
(d) obtaining a second biological sample after administration of the TREM2 agonist to the patient; and
(e) measuring a level in the second biological sample of one or more biomarkers selected from APOE, B2M, BIRC5, BST2, C1QA/B/C, CCL12, CCL2, CCL3, CCL4, CCNB2, CD3G, CD63, CD74, CD81, CD9, CST7, CTSB, CXCL10, CXCL2, FTL1, H2-AA, H2-AB1, H2AFV, H2AFZ, H2-D1, H2-DMA, H2-DMB1, H2-DMB2, H2-EB1, H2-K1, H2-OA, H2-OB, H2-Q10, H2-Q4, H2-Q6, H2-Q7, H2-T23, HEXB, HMGB2, HMGN2, IFI204, IFI2712A, IFIT3, IFITM3, IL1B, IRF7, ISG15, LGALS3BP, LGMN, LPL, LY6E, MLF, MR1, MRC1, MS4A4B, OASL2, OLFML3, P2RY12, PF4, RTP4, SLFN2, SPARC, STMN1, TMEM119, TUBA1B, TUBB5, or USP18;
   wherein when the level of one or more biomarkers selected from APOE, B2M, BIRC5, BST2, C1QA/B/C, CCL12, CCL2, CCL3, CCL4, CCNB2, CD3G, CD63, CD74, CD81, CD9, CST7, CTSB, CXCL10, CXCL2, FTL1, H2-AA, H2-AB1, H2AFV, H2AFZ, H2-D1, H2-DMA, H2-DMB1, H2-DMB2, H2-EB1, H2-K1, H2-OA, H2-OB, H2-Q10, H2-Q4, H2-Q6, H2-Q7, H2-T23, HEXB, HMGB2, HMGN2, IFI204, IFI2712A, IFIT3, IFITM3, IL1B, IRF7, ISG15, LGALS3BP, LGMN, LPL, LY6E, MLF, MR1, MRC1, MS4A4B, OASL2, OLFML3, P2RY12, PF4, RTP4, SLFN2, SPARC, STMN1, TMEM119, TUBA1B, TUBB5, or USP18 is higher in the second biological sample from the patient than in the first biological sample from the patient, then the patient is administered one or more additional doses of the TREM2 agonist.

In certain embodiments, the one or more biomarkers are selected from CCL2, CCL4, CST7, CXCL2, CXCL10, IL1B, or TMEM119. In certain embodiments, the TREM2 agonist is an anti-hTREM2 antibody. In some embodiments, steps (b) and (e) further comprise measuring the level of one or more biomarkers selected from those listed in Table A.

In another aspect, the present invention provides a method of evaluating a patient response to a TREM2 agonist, comprising the steps of:
(a) obtaining a first biological sample from the patient prior to administration of the TREM2 agonist to the patient;
(b) measuring a level in the first biological sample of one or more biomarkers selected from APOE, B2M, BIRC5, BST2, C1QA/B/C, CCL12, CCL2, CCL3, CCL4, CCNB2, CD3G, CD63, CD74, CD81, CD9, CST7, CTSB, CXCL10, CXCL2, FTL1, H2-AA, H2-AB1, H2AFV, H2AFZ, H2-D1, H2-DMA, H2-DMB1, H2-DMB2, H2-EB1, H2-K1, H2-OA, H2-OB, H2-Q10, H2-Q4, H2-Q6, H2-Q7, H2-T23, HEXB, HMGB2, HMGN2, IFI204, IFI2712A, IFIT3, IFITM3, IL1B, IRF7, ISG15, LGALS3BP, LGMN, LPL, LY6E, MLF, MR1, MRC1, MS4A4B, OASL2, OLFML3, P2RY12, PF4, RTP4, SLFN2, SPARC, STMN1, TMEM119, TUBA1B, TUBB5, or USP18;
(c) administering to the patient an effective amount of a TREM2 agonist;
(d) obtaining a second biological sample after administration of the TREM2 agonist to the patient; and
(e) measuring a level in the second biological sample of one or more biomarkers selected from APOE, B2M, BIRC5, BST2, C1QA/B/C, CCL12, CCL2, CCL3, CCL4, CCNB2, CD3G, CD63, CD74, CD81, CD9, CST7, CTSB, CXCL10, CXCL2, FTL1, H2-AA, H2-AB1, H2AFV, H2AFZ, H2-D1, H2-DMA, H2-DMB1, H2-DMB2, H2-EB1, H2-K1, H2-OA, H2-OB, H2-Q10, H2-Q4, H2-Q6, H2-Q7, H2-T23, HEXB, HMGB2, HMGN2, IFI204, IFI2712A, IFIT3, IFITM3, IL1B, IRF7, ISG15, LGALS3BP, LGMN, LPL, LY6E, MLF, MR1, MRC1, MS4A4B, OASL2, OLFML3, P2RY12, PF4, RTP4, SLFN2, SPARC, STMN1, TMEM119, TUBA1B, TUBB5, or USP18;
   wherein the Alzheimer's disease response to step (c) is evaluated to split, classify, or stratify the patient into one of two or more groups based on a greater or lesser response of the plaque compared with one or more similar patients.

In certain embodiments, the one or more biomarkers are selected from CCL2, CCL4, CST7, CXCL2, CXCL10, IL1B, or TMEM119. In certain embodiments, the TREM2 agonist is an anti-hTREM2 antibody. In some embodiments, steps (b) and (e) further comprise measuring the level of one or more biomarkers selected from those listed in Table A.

In another aspect, the present invention provides a method of predicting a patient response to a TREM2 agonist, comprising the steps of:
(a) obtaining a first biological sample from the patient prior to administration of the TREM2 agonist to the patient;
(b) measuring a level in the first biological sample from the patient of one or more biomarkers selected from APOE, B2M, BIRC5, BST2, C1QA/B/C, CCL12, CCL2, CCL3, CCL4, CCNB2, CD3G, CD63, CD74, CD81, CD9, CST7, CTSB, CXCL10, CXCL2, FTL1, H2-AA, H2-AB1, H2AFV, H2AFZ, H2-D1, H2-DMA, H2-DMB1, H2-DMB2, H2-EB1, H2-K1, H2-OA, H2-OB, H2-Q10, H2-Q4, H2-Q6, H2-Q7, H2-T23, HEXB, HMGB2, HMGN2, IFI204, IFI2712A, IFIT3, IFITM3, IL1B, IRF7, ISG15, LGALS3BP, LGMN, LPL, LY6E, MLF, MR1, MRC1, MS4A4B, OASL2, OLFML3, P2RY12, PF4, RTP4, SLFN2, SPARC, STMN1, TMEM119, TUBA1B, TUBB5, or USP18;
(c) administering to the patient an effective amount of a TREM2 agonist;
(d) obtaining a second biological sample from the patient after administration of the TREM2 agonist to the patient; and
(e) measuring a level in the second biological sample from the patient of one or more biomarkers selected from APOE, B2M, BIRC5, BST2, C1QA/B/C, CCL12, CCL2, CCL3, CCL4, CCNB2, CD3G, CD63, CD74, CD81, CD9, CST7, CTSB, CXCL10, CXCL2, FTL1, H2-AA, H2-AB1, H2AFV, H2AFZ, H2-D1, H2-DMA, H2-DMB1, H2-DMB2, H2-EB1, H2-K1, H2-OA, H2-OB, H2-Q10, H2-Q4, H2-Q6, H2-Q7, H2-T23, HEXB, HMGB2, HMGN2, IFI204, IFI2712A, IFIT3, IFITM3, IL1B, IRF7, ISG15, LGALS3BP, LGMN, LPL, LY6E, MLF, MR1, MRC1, MS4A4B, OASL2, OLFML3, P2RY12, PF4, RTP4, SLFN2, SPARC, STMN1, TMEM119, TUBA1B, TUBB5, or USP18;
   wherein the Alzheimer's disease response to step (c) is predictive of the likelihood of successful treatment based on a greater or lesser response of the Alzheimer's disease compared with one or more similar patients and as evaluated using one or more of the biomarkers.

In certain embodiments, the one or more biomarkers are selected from CCL2, CCL4, CST7, CXCL2, CXCL10, IL1B, or TMEM119. In certain embodiments, the TREM2 agonist is an anti-hTREM2 antibody. In some embodiments, steps (b) and (e) further comprise measuring the level of one or more biomarkers selected from those listed in Table A.

In another aspect, the present invention provides a method of predicting a treatment response of Alzheimer's disease in a patient to a TREM2 agonist, comprising the steps of:
(a) obtaining a first biological sample from the patient prior to administration of the TREM2 agonist to the patient;
(b) measuring a level in the first biological sample of one or more biomarkers selected from APOE, B2M, BIRC5, BST2, C1QA/B/C, CCL12, CCL2, CCL3, CCL4, CCNB2, CD3G, CD63, CD74, CD81, CD9, CST7, CTSB, CXCL10, CXCL2, FTL1, H2-AA, H2-AB1, H2AFV, H2AFZ, H2-D1, H2-DMA, H2-DMB1, H2-DMB2, H2-EB1, H2-K1, H2-OA, H2-OB, H2-Q10, H2-Q4, H2-Q6, H2-Q7, H2-T23, HEXB, HMGB2, HMGN2, IFI204, IFI2712A, IFIT3, IFITM3, IL1B, IRF7, ISG15, LGALS3BP, LGMN, LPL, LY6E, MLF, MR1, MRC1, MS4A4B, OASL2, OLFML3, P2RY12, PF4, RTP4, SLFN2, SPARC, STMN1, TMEM119, TUBA1B, TUBB5, or USP18;
(c) treating the biological sample from the patient or a reference sample;
(d) measuring a level in the treated biological sample of one or more biomarkers selected from APOE, B2M, BIRC5, BST2, C1QA/B/C, CCL12, CCL2, CCL3, CCL4, CCNB2, CD3G, CD63, CD74, CD81, CD9, CST7, CTSB, CXCL10, CXCL2, FTL1, H2-AA, H2-AB1, H2AFV, H2AFZ, H2-D1, H2-DMA, H2-DMB1, H2-DMB2, H2-EB1, H2-K1, H2-OA, H2-OB, H2-Q10, H2-Q4, H2-Q6, H2-Q7, H2-T23, HEXB, HMGB2, HMGN2, IFI204, IFI2712A, IFIT3, IFITM3, IL1B, IRF7, ISG15, LGALS3BP, LGMN, LPL, LY6E, MLF, MR1, MRC1, MS4A4B, OASL2, OLFML3, P2RY12, PF4, RTP4, SLFN2, SPARC, STMN1, TMEM119, TUBA1B, TUBB5, or USP18;
(e) comparing one of more biomarkers in the pre-treatment biological sample with one or more biomarkers in the treated biological sample or treated reference sample; and
(f) optionally, proceeding with administration of the TREM2 agonist to the patient, if such administration is predicted to have an equivalent or higher likelihood of success relative to an alternative method of treating the Alzheimer's disease;
   wherein the biomarker change in response to step (c) is predictive of the likelihood of successful treatment of the Alzheimer's disease based on a greater or lesser biomarker change compared with one or more similar patients and as evaluated using one or more of the biomarkers.

In certain embodiments, the one or more biomarkers are selected from CCL2, CCL4, CST7, CXCL2, CXCL10, IL1B, or TMEM119. In certain embodiments, the TREM2 agonist is an anti-hTREM2 antibody. In some embodiments, steps (b) and (d) further comprise measuring the level of one or more biomarkers selected from those listed in Table A.

In some embodiments, the reference sample is from another patient, such as a patient with a similar pathology of Alzheimer's disease; or the reference sample may be a culture or other in vitro sample of a similar pathology of Alzheimer's disease.

In another aspect, the present invention provides a method of monitoring a patient response to a TREM2 agonist, comprising the steps of:
(a) obtaining a first biological sample from the patient prior to administration of the TREM2 agonist to the patient;
(b) measuring a level in the first biological sample from the patient of one or more biomarkers selected from APOE, B2M, BIRC5, BST2, C1QA/B/C, CCL12, CCL2, CCL3, CCL4, CCNB2, CD3G, CD63, CD74, CD81, CD9, CST7, CTSB, CXCL10, CXCL2, FTL1, H2-AA, H2-AB1, H2AFV, H2AFZ, H2-D1, H2-DMA, H2-DMB1, H2-DMB2, H2-EB1, H2-K1, H2-OA, H2-OB, H2-Q10, H2-Q4, H2-Q6, H2-Q7, H2-T23, HEXB, HMGB2, HMGN2, IFI204, IFI2712A, IFIT3, IFITM3, IL1B, IRF7, ISG15, LGALS3BP, LGMN, LPL, LY6E, MLF, MR1, MRC1, MS4A4B, OASL2, OLFML3, P2RY12, PF4, RTP4, SLFN2, SPARC, STMN1, TMEM119, TUBA1B, TUBB5, or USP18;
(c) administering to the patient an effective amount of a TREM2 agonist;
(d) obtaining a one or more subsequent biological samples from the patient after administration of the TREM2 agonist to the patient; and
(e) measuring a level in the subsequent biological sample(s) of one or more biomarkers selected from APOE, B2M, BIRC5, BST2, C1QA/B/C, CCL12, CCL2, CCL3, CCL4, CCNB2, CD3G, CD63, CD74, CD81, CD9, CST7, CTSB, CXCL10, CXCL2, FTL1, H2-AA, H2-AB1, H2AFV, H2AFZ, H2-D1, H2-DMA, H2-DMB1, H2-DMB2, H2-EB1, H2-K1, H2-OA, H2-OB, H2-Q10, H2-Q4, H2-Q6, H2-Q7, H2-T23, HEXB, HMGB2, HMGN2, IFI204, IFI2712A, IFIT3, IFITM3, IL1B, IRF7, ISG15, LGALS3BP, LGMN, LPL, LY6E, MLF, MR1, MRC1, MS4A4B, OASL2, OLFML3, P2RY12, PF4, RTP4, SLFN2, SPARC, STMN1, TMEM119, TUBA1B, TUBB5, or USP18;
   wherein the levels of one of more biomarkers in the first biological sample and subsequent biological samples can be compared and changes in one or more of the biomarkers indicate a patient response.

In certain embodiments, the one or more biomarkers are selected from CCL2, CCL4, CST7, CXCL2, CXCL10, IL1B, or TMEM119. In certain embodiments, the TREM2 agonist is an anti-hTREM2 antibody. In some embodiments, steps (b) and (e) further comprise measuring the level of one or more biomarkers selected from those listed in Table A.

In some embodiments, the patient response to a TREM2 agonist is measured once per week or every two weeks. In some embodiments, the patient response is measured once a month. In some embodiments, the patient's response is measured bimonthly. In some embodiments, the patient's response is measured quarterly (once every three months). In some embodiments, the patient's response is measured annually.

In some embodiments, the patient response to a TREM2 agonist is monitored while undergoing treatment. In some embodiments, the patient response is monitored after treatment is concluded.

In another aspect, the present invention provides a method of deriving a biomarker signature that is predictive of response to treatment with a TREM2 agonist, comprising:
(a) obtaining a pre-treatment biological sample from each patient in a patient cohort diagnosed with Alzheimer's disease;
(b) obtaining, for each patient in the cohort, a response value following treatment with the a TREM2 agonist;
(c) measuring the raw biomarker levels in each biological sample for each gene in a biomarker platform, wherein the biomarker platform comprises a clinical response biomarker set of APOE, B2M, BIRC5, BST2, C1QA/B/C, CCL12, CCL2, CCL3, CCL4, CCNB2, CD3G, CD63, CD74, CD81, CD9, CST7, CTSB, CXCL10, CXCL2, FTL1, H2-AA, H2-AB1, H2AFV, H2AFZ, H2-D1, H2-DMA, H2-DMB1, H2-DMB2, H2-EB1, H2-K1, H2-OA, H2-OB, H2-Q10, H2-Q4, H2-Q6, H2-Q7, H2-T23, HEXB, HMGB2, HMGN2, IFI204, IFI2712A, IFIT3, IFITM3, IL1B, IRF7, ISG15, LGALS3BP, LGMN, LPL, LY6E, MLF, MR1, MRC1, MS4A4B, OASL2, OLFML3, P2RY12, PF4, RTP4, SLFN2, SPARC, STMN1, TMEM119, TUBA1B, TUBB5, or USP18;
(d) normalizing, for each biological sample, each of the measured raw biomarker levels for the clinical response biomarkers using the measured biomarker levels of a set of normalization biomarkers; and
(e) comparing the biomarker levels for all of the biological samples and the response values for all of the patients in the cohort to select a cutoff for the biomarker signature score that divides the patient cohort to meet a target biomarker clinical utility criterion.

In some embodiments, the biomarker platform comprises a gene expression platform that comprises a clinical response gene set. In some embodiments, the method further comprises the steps of:
(f) weighting, for each biological sample and each biomarker, such as a gene in a gene signature of interest, the normalized biomarker (e.g., RNA biomarker) expression levels using a pre-defined multiplication coefficient for that gene;
(g) adding, for each patient, the weighted biomarker (e.g., RNA biomarker) expression levels to generate a biomarker signature score, e.g., a gene signature score, for each patient in the cohort.

In certain embodiments, the one or more biomarkers are selected from CCL2, CCL4, CST7, CXCL2, CXCL10, IL1B, or TMEM119. In certain embodiments, the TREM2 agonist is an anti-hTREM2 antibody. In some embodiments, the biomarker platform further comprises one or more biomarkers selected from those listed in Table A.

In another aspect, the present invention provides a method of testing a biological sample from a patient for the presence or absence of a gene signature biomarker of response of Alzheimer's disease to a TREM2 agonist, comprising:
(a) measuring the raw RNA level in the biological sample for each gene in a gene expression platform, wherein the gene expression platform comprises a clinical response gene set selected from an APOE, B2M, BIRC5, BST2, C1QA/B/C, CCL12, CCL2, CCL3, CCL4, CCNB2, CD3G, CD63, CD74, CD81, CD9, CST7, CTSB, CXCL10, CXCL2, FTL1, H2-AA, H2-AB1, H2AFV, H2AFZ, H2-D1, H2-DMA, H2-DMB1, H2-DMB2, H2-EB1, H2-K1, H2-OA, H2-OB, H2-Q10, H2-Q4, H2-Q6, H2-Q7, H2-T23, HEXB, HMGB2, HMGN2, IFI204, IFI2712A, IFIT3, IFITM3, IL1B, IRF7, ISG15, LGALS3BP, LGMN, LPL, LY6E, MLF, MR1, MRC1, MS4A4B, OASL2, OLFML3, P2RY12, PF4, RTP4, SLFN2, SPARC, STMN1, TMEM119, TUBA1B, TUBB5, or USP18 and a normalization gene set of housekeeping genes, and optionally wherein about 80%, or about 90%, of the clinical response genes exhibit RNA levels that are positively correlated with the Alzheimer's disease response;
(b) normalizing the measured raw RNA level for each clinical response gene in a pre-defined gene signature for the biological sample using the measured RNA levels of the normalization genes, wherein the pre-defined gene signature consists of at least 2 of the clinical response genes, thus obtaining a gene signature score;
(c) comparing the gene signature score to a reference score for the gene signature; and (d) classifying the biological sample as biomarker high or biomarker low;
   wherein if the generated score is equal to or greater than the reference score, then the biological sample is classified as biomarker high, and if the generated score is less than the reference score, then the biological sample is classified as biomarker low.

In certain embodiments, the one or more biomarkers are selected from CCL2, CCL4, CST7, CXCL2, CXCL10, IL1B, or TMEM119. In certain embodiments, the TREM2 agonist is an anti-hTREM2 antibody. In some embodiments, the gene expression platform further comprises one or more biomarkers selected from those listed in Table A.

In some embodiments, after step (b) the method comprises the further steps of:
(i) weighting each normalized RNA value using a pre-defined multiplication co-efficient;
(ii) adding the weighted RNA expression levels to generate a weighted gene signature score.

In some embodiments, the normalization gene set comprises about 1 to 5 housekeeping genes, 5 to 10 housekeeping genes, 10 to about 20 housekeeping genes, or about 30-40 housekeeping genes.

In another aspect, the present invention provides a method of testing a biological sample from a patient diagnosed with Alzheimer's for the presence or absence of a biomarker signature of response of the Alzheimer's disease to a TREM2 agonist, comprising:
(a) measuring the raw biomarker level in the biological sample for each biomarker in a biomarker platform, wherein the biomarker platform comprises a clinical response biomarker set selected from APOE, B2M, BIRC5, BST2, C1QA/B/C, CCL12, CCL2, CCL3, CCL4, CCNB2, CD3G, CD63, CD74, CD81, CD9, CST7, CTSB, CXCL10, CXCL2, FTL1, H2-AA, H2-AB1, H2AFV, H2AFZ, H2-D1, H2-DMA, H2-DMB1, H2-DMB2, H2-EB1, H2-K1, H2-OA, H2-OB, H2-Q10, H2-Q4, H2-Q6, H2-Q7, H2-T23, HEXB, HMGB2, HMGN2, IFI204, IFI2712A, IFIT3, IFITM3, IL1B, IRF7, ISG15, LGALS3BP, LGMN, LPL, LY6E, MLF, MR1, MRC1, MS4A4B, OASL2, OLFML3, P2RY12, PF4, RTP4, SLFN2, SPARC, STMN1, TMEM119, TUBA1B, TUBB5, or USP18 and a normalization biomarker set, and optionally wherein about 80%, or about 90%, of the clinical response biomarkers exhibit levels that are positively correlated with the Alzheimer's disease response;
(b) normalizing the measured raw biomarker level for each clinical response biomarker in a pre-defined biomarker signature for the biological sample using the measured biomarker levels of the normalization biomarkers, wherein the pre-defined biomarker signature consists of at least 2 of the clinical response biomarkers;
(c) comparing the normalized biomarker levels and a set of reference biomarker levels for the biological sample; and
(d) classifying the biological sample as biomarker high or biomarker low;
   wherein if the normalized biomarker levels are equal to or greater than the reference biomarker levels, then the biological sample is classified as biomarker high, and if the normalized biomarker levels are less than the reference biomarker levels, then the biological sample is classified as biomarker low.

In certain embodiments, the one or more biomarkers are selected from CCL2, CCL4, CST7, CXCL2, CXCL10, IL1B, or TMEM119. In certain embodiments, the TREM2 agonist is an anti-hTREM2 antibody. In some embodiments, the biomarker platform further comprises one or more biomarkers selected from those listed in Table A.

In some embodiments, the normalization biomarker set comprises about 10 to about 12 housekeeping genes, or about 30-40 housekeeping genes.

In another aspect, the present invention provides a system for testing a sample of a biological sample removed from a patient having Alzheimer's disease for the presence or absence of a biomarker signature of response to a TREM2 agonist, comprising:
(i) a sample analyzer for measuring raw biomarker levels in a biomarker platform, wherein the biomarker platform consists of a set of clinical response biomarkers selected from APOE, B2M, BIRC5, BST2, C1QA/B/C, CCL12, CCL2, CCL3, CCL4, CCNB2, CD3G, CD63, CD74, CD81, CD9, CST7, CTSB, CXCL10, CXCL2, FTL1, H2-AA, H2-AB1, H2AFV, H2AFZ, H2-D1, H2-DMA, H2-DMB1, H2-DMB2, H2-EB1, H2-K1, H2-OA, H2-OB, H2-Q10, H2-Q4, H2-Q6, H2-Q7, H2-T23, HEXB, HMGB2, HMGN2, IFI204, IFI2712A, IFIT3, IFITM3, IL1B, IRF7, ISG5, LGALS3BP, LGMN, LPL, LY6E, MLF, MR1, MRC1, MS4A4B, OASL2, OLFML3, P2RY12, PF4, RTP4, SLFN2, SPARC, STMN1, TMEM119, TUBA1B, TUBB5, or USP18; and a set of normalization biomarkers; and
(ii) a computer program for receiving and analyzing the measured biomarker levels to:
   (a) normalize the measured raw biomarker level for each clinical response biomarker in a pre-defined biomarker signature for the biological sample using the measured levels of the normalization biomarkers;
   (b) compare the generated biomarker level to a reference level for the biomarker signature; and
   (c) classify the biological sample as biomarker high or biomarker low, wherein if the generated score is equal to or greater than the reference score, then the biological sample is classified as biomarker high, and if the generated score is less than the reference score, then the biological sample is classified as biomarker low.

In certain embodiments, the one or more biomarkers are selected from CCL2, CCL4, CST7, CXCL2, CXCL10, IL1B, or TMEM119. In certain embodiments, the TREM2 agonist is an anti-hTREM2 antibody. In some embodiments, the biomarker platform further includes one or more biomarkers selected from those listed in Table A.

In another aspect, the present invention provides a system for testing a biological sample from a patient diagnosed with Alzheimer's disease for the presence or absence of a biomarker signature of response of the Alzheimer's disease to a TREM2 agonist, comprising:
(i) a sample analyzer for measuring raw biomarker levels in a biomarker platform, wherein the biomarker platform consists of a set of clinical response biomarkers selected from APOE, B2M, BIRC5, BST2, C1QA/B/C, CCL12, CCL2, CCL3, CCL4, CCNB2, CD3G, CD63, CD74, CD81, CD9, CST7, CTSB, CXCL10, CXCL2, FTL1, H2-AA, H2-AB1, H2AFV, H2AFZ, H2-D1, H2-DMA, H2-DMB1, H2-DMB2, H2-EB1, H2-K1, H2-OA, H2-OB, H2-Q10, H2-Q4, H2-Q6, H2-Q7, H2-T23, HEXB, HMGB2, HMGN2, IFI204, IFI2712A, IFIT3, IFITM3, IL1B, IRF7, ISG5, LGALS3BP, LGMN, LPL, LY6E, MLF, MR1, MRC1, MS4A4B, OASL2, OLFML3, P2RY12, PF4, RTP4, SLFN2, SPARC, STMN1, TMEM119, TUBA1B, TUBB5, or USP18; and a set of normalization biomarkers; and
(ii) a computer program for receiving and analyzing the measured biomarker levels to
   (a) normalize the measured raw biomarker level for each clinical response biomarker in a pre-defined biomarker signature for the biological sample using the measured levels of the normalization biomarkers;
   (b) weight each normalized biomarker level using a pre-defined multiplication coefficient;
   (c) add the weighted biomarker levels to generate a biomarker signature score;
   (d) compare the generated score to a reference score for the biomarker signature; and
   (e) classify the biological sample as biomarker high or biomarker low, wherein if the generated score is equal to or greater than the reference score, then the biological sample is classified as biomarker high, and if the generated score is less than the reference score, then the biological sample is classified as biomarker low.

In certain embodiments, the one or more biomarkers are selected from CCL2, CCL4, CST7, CXCL2, CXCL10, IL1B, or TMEM119. In certain embodiments, the TREM2 agonist is an anti-hTREM2 antibody. In some embodiments, the biomarker platform further includes one or more biomarkers selected from those listed in Table A.

In some embodiments, the biomarker comprises the RNA expression level of a gene described herein, such as APOE, B2M, BIRC5, BST2, C1QA/B/C, CCL12, CCL2, CCL3, CCL4, CCNB2, CD3G, CD63, CD74, CD81, CD9, CST7, CTSB, CXCL10, CXCL2, FTL1, H2-AA, H2-AB1, H2AFV, H2AFZ, H2-D1, H2-DMA, H2-DMB1, H2-DMB2, H2-EB1, H2-K1, H2-OA, H2-OB, H2-Q10, H2-Q4, H2-Q6, H2-Q7, H2-T23, HEXB, HMGB2, HMGN2, IFI204, IFI2712A, IFIT3, IFITM3, IL1B, IRF7, ISG15, LGALS3BP, LGMN, LPL, LY6E, MLF, MR1, MRC1, MS4A4B, OASL2, OLFML3, P2RY12, PF4, RTP4, SLFN2, SPARC, STMN1, TMEM119, TUBA1B, TUBB5, or USP18. In some embodiments, the biomarker further comprises the RNA expression level of one or more genes listed in Table A. In certain embodiments, the one or more biomarkers are selected from Ccl2, Ccl4, Cxcl10, Cst7, or Tmem119.

In another aspect, the present invention provides a kit for assaying a biological sample from a an Alzheimer's patient treated with a TREM2 agonist to obtain normalized RNA expression scores for a gene signature associated with the plaque microenvironment, wherein the kit comprises:
(a) a set of hybridization probes capable of specifically binding to a transcript expressed by each of the genes; and
(b) a set of reagents designed to quantify the number of specific hybridization complexes formed with each hybridization probe.

In some embodiments, the gene signature is selected from two or more of APOE, B2M, BIRC5, BST2, C1QA/B/C, CCL12, CCL2, CCL3, CCL4, CCNB2, CD3G, CD63, CD74, CD81, CD9, CST7, CTSB, CXCL10, CXCL2, FTL1, H2-AA, H2-AB1, H2AFV, H2AFZ, H2-D1, H2-DMA, H2-DMB1, H2-DMB2, H2-EB1, H2-K1, H2-OA, H2-OB, H2-Q10, H2-Q4, H2-Q6, H2-Q7, H2-T23, HEXB, HMGB2, HMGN2, IFI204, IFI2712A, IFIT3, IFITM3, IL1B, IRF7, ISG15, LGALS3BP, LGMN, LPL, LY6E, MLF, MR1, MRC1, MS4A4B, OASL2, OLFML3, P2RY12, PF4, RTP4, SLFN2, SPARC, STMN1, TMEM119, TUBA1B, TUBB5, or USP18. In some embodiments, the gene signature further comprises one or more biomarkers selected from those listed in Table A.

In certain embodiments, the one or more biomarkers are selected from CCL2, CCL4, CST7, CXCL2, CXCL10, IL1B, or TMEM119. In certain embodiments, the TREM2 agonist is an anti-hTREM2 antibody.

In another aspect, the present invention provides a method for treating a patient having Alzheimer's disease, comprising determining if a biological sample from the patient is positive or negative for a biomarker such as a gene signature biomarker and administering to the patient a TREM2 agonist if the biological sample is positive for the biomarker and administering to the subject an Alzheimer's disease treatment that does not include a TREM2 agonist if the biological sample from the patient is negative for the biomarker, wherein the biomarker such as gene signature biomarker is for a biomarker, e.g. gene signature biomarker, that comprises at least two of the clinical response biomarkers selected from APOE, B2M, BIRC5, BST2, C1QA/B/C, CCL12, CCL2, CCL3, CCL4, CCNB2, CD3G, CD63, CD74, CD81, CD9, CST7, CTSB, CXCL10, CXCL2, FTL1, H2-AA, H2-AB1, H2AFV, H2AFZ, H2-D1, H2-DMA, H2-DMB1, H2-DMB2, H2-EB1, H2-K1, H2-OA, H2-OB, H2-Q10, H2-Q4, H2-Q6, H2-Q7, H2-T23, HEXB, HMGB2, HMGN2, IFI204, IFI2712A, IFIT3, IFITM3, IL1B, IRF7, ISG15, LGALS3BP, LGMN, LPL, LY6E, MLF, MR1, MRC1, MS4A4B, OASL2, OLFML3, P2RY12, PF4, RTP4, SLFN2, SPARC, STMN1, TMEM119, TUBA1B, TUBB5, or USP18. In some embodiments, a multi-gene signature score, such as an CCL2, CCL4, CST7, CXCL2, CXCL10, IL1B, or TMEM119 signature score can be used as one “biomarker” in the same grouping as other individual gene biomarkers, to calculate a more predictive gene signature score. In some embodiments, the clinical response biomarkers further comprise one or more biomarkers listed in Table A.

In another aspect, the present invention provides a method of testing a biological sample from a patient to generate a signature score for a gene signature that is correlated with an Alzheimer's disease response to a TREM2 agonist, wherein the method comprises:
(a) measuring the raw RNA level in the biological sample for each gene in the gene signature and for each gene in a normalization gene set, wherein the gene signature comprises genes selected from APOE, B2M, BIRC5, BST2, C1QA/B/C, CCL12, CCL2, CCL3, CCL4, CCNB2, CD3G, CD63, CD74, CD81, CD9, CST7, CTSB, CXCL10, CXCL2, FTL1, H2-AA, H2-AB1, H2AFV, H2AFZ, H2-D1, H2-DMA, H2-DMB1, H2-DMB2, H2-EB1, H2-K1, H2-OA, H2-OB, H2-Q10, H2-Q4, H2-Q6, H2-Q7, H2-T23, HEXB, HMGB2, HMGN2, IFI204, IFI2712A, IFIT3, IFITM3, IL1B, IRF7, ISG15, LGALS3BP, LGMN, LPL, LY6E, MLF, MR1, MRC1, MS4A4B, OASL2, OLFML3, P2RY12, PF4, RTP4, SLFN2, SPARC, STMN1, TMEM119, TUBA1B, TUBB5, or USP18;
(b) normalizing the measured raw RNA level for each gene in the gene signature using the measured RNA levels of the normalization genes;
(c) multiplying each normalized RNA value by a calculated scoring weight set to generate a weighted RNA expression value; and
(d) adding the weighted RNA expression values to generate the gene signature score.

In certain embodiments, the one or more biomarkers are selected from CCL2, CCL4, CST7, CXCL2, CXCL10, IL1B, or TMEM119. In certain embodiments, the TREM2 agonist is an anti-hTREM2 antibody.

In some embodiments, a multi-gene signature score, such as an interferon signature score, can be used as one “biomarker” in the same grouping as other individual gene biomarkers, to calculate a more predictive gene signature score.

In some embodiments of the invention, the measuring step comprises isolating RNA from the biological sample from the patient and incubating the sample with a set of probes that are designed to specifically hybridize to gene target regions of the RNA. In some embodiments, the first biological sample from the patient and/or second biological sample from the patient are assayed in vitro or ex vivo.

In some embodiments of any of the aforementioned methods, the method further comprises measuring the level of one or more additional biomarker, or gene expression level as appropriate to the method, selected from those listed in Table A. In any of the aforementioned embodiments, during any step wherein the level of a biomarker is measured, said step optionally further comprises measuring one or more additional biomarkers selected from those listed in Table A. In some embodiments, the method optionally further comprises measuring two or more additional biomarkers selected from those listed in Table A. In some embodiments, the method optionally further comprises measuring three or more additional biomarkers selected from those listed in Table A.

In another aspect, the present invention provides a method of inducing microglial activation in a patient towards specific microglia cell type trajectories, comprising administering to the patient an effective amount of a TREM2 agonist. In some embodiments, microglial activation is towards a disease-associated (DAM) microglia type trajectory. In some embodiments, microglial activation is towards an interferon-responsive (IFN-R) microglia type trajectory. In some embodiments, microglial activation is towards a cycling (Cyc-M) microglia type trajectory. In some embodiments, microglial activation is towards an MHC-II expressing (MHC-II) microglia type trajectory. In some embodiments, the patient is diagnosed with Alzheimer's disease. In some embodiments, the TREM2 agonist is an anti-hTREM2 antibody.

### 6.5. Binding Targets

The present invention is directed to the use of therapeutic molecules that specifically bind to TREM2, particularly human TREM2. TREM2 is a member of the Ig superfamily of receptors that is expressed on cells of myeloid lineage, including macrophages, dendritic cells, and microglia (Schmid et al., Journal of Neurochemistry, Vol. 83: 1309-1320, 2002; Colonna, Nature Reviews Immunology, Vol. 3: 445-453, 2003; Kiialainen et al., Neurobiology of Disease, 2005, 18: 314-322). TREM2 is an immune receptor that binds many endogenous substrates, including ApoE, LPS, exposed phospholipids, phosphatidylserine and amyloid beta and signals through a short intracellular domain that complexes with the adaptor protein DAP12, the cytoplasmic domain of which comprises an ITAM motif (Bouchon et al., The Journal of Experimental Medicine, 2001, 194: 1111-1122). Upon activation of TREM2, tyrosine residues within the ITAM motif in DAP12 are phosphorylated by the Src family of kinases, providing docking sites for the tyrosine kinase ζ-chain-associated protein 70 (ZAP70) and spleen tyrosine kinase (Syk) via their SH2 domains (Colonna, Nature Reviews Immunology, 2003, 3:445-453; Ulrich and Holtzman, ACS Chem. Neurosci., 2016, 7:420-427). The ZAP70 and Syk kinases induce activation of several downstream signaling cascades, including phosphatidylinositol 3-kinase (PI3K), protein kinase C (PKC), extracellular regulated kinase (ERK), and elevation of intracellular calcium (Colonna, Nature Reviews Immunology, 2003, 3:445-453; Ulrich and Holtzman, ACS Chem. Neurosci., 2016, 7:420-427).

TREM2 has been implicated in several myeloid cell processes, including phagocytosis, proliferation, survival, and regulation of inflammatory cytokine production (Ulrich and Holtzman, ACS Chem. Neurosci., 2016, 7: 420-427). In the last few years, TREM2 has been linked to several diseases. For instance, mutations in both TREM2 and DAP12 have been linked to the autosomal recessive disorder Nasu-Hakola Disease, which is characterized by bone cysts, muscle wasting and demyelination phenotypes (Guerreiro et al., New England Journal of Medicine, 2013, 368: 117-127). More recently, variants in the TREM12 gene have been linked to increased risk for Alzheimer's disease (AD) and other forms of dementia including frontotemporal dementia and amyotrophic lateral sclerosis (Jonsson et al., New England Journal of Medicine, 2013, 368:107-116; Guerreiro et al., JAMA Neurology, 2013, 70:78-84; Jay et al., Journal of Experimental Medicine, 2015, 212: 287-295; Cady et al, JAMA Neurol. 2014 April; 71(4):449-53). In particular, the R47H variant has been identified in genome-wide studies as being associated with increased risk for late-onset AD with an overall adjusted odds ratio (for populations of all ages) of 2.3, second only to the strong genetic association of ApoE to Alzheimer's. The R47H mutation resides on the extracellular Ig V-set domain of the TREM2 protein and has been shown to impact lipid binding and uptake of apoptotic cells and Abeta (Wang et al., Cell, 2015, 160: 1061-1071; Yeh et al., Neuron, 2016, 91: 328-340), suggestive of a loss-of-function linked to disease. Further, postmortem comparison of AD patients' brains with and without the R47H mutation are supportive of a novel loss-of-microglial barrier function for the carriers of the mutation, with the R47H carrier microglia putatively demonstrating a reduced ability to compact plaques and limit their spread (Yuan et al., Neuron, 2016, 90: 724-739). Impairment in microgliosis has been reported in animal models of prion disease, multiple sclerosis, and stroke, suggesting that TREM2 may play an important role in supporting microgliosis in response to pathology or damage in the central nervous system (Ulrich and Holtzman, ACS Chem. Neurosci., 2016, 7: 420-427).

In humans, the TREM2 gene is located within a TREM gene cluster at chromosome 6p21.1. The TREM gene cluster encodes four TREM proteins (TREM1, TREM2, TREM4, and TREM5) as well as two TREM-like proteins (TLT-1 and TLT-2). The TREM2 gene encodes a 230 amino acid protein consisting of an extracellular domain, a transmembrane region, and a short cytoplasmic tail (Paradowska-Gorycka et ah, Human Immunology, Vol. 74: 730-737, 2013). The extracellular domain contains a single type V Ig-super family domain, with three potential N-glycosylation sites. The 230 amino acid wild-type hTREM2 amino acid sequence (NCBI Reference Sequence: NP_061838.1) is provided below as SEQ ID NO: 1.

| (SEQ ID NO: 1) | |
|---|---|
| 1 | MEPLRLLILLFVTELSGAHNTTVFQGVAGQSLQVSCPYDSMKHWGRRKAWCRQLGEKGPC |
| 61 | QRVVSTHNLWLLSFLRRWNGSTAITDDTLGGTLTITLRNLQPHDAGLYQCQSLHGSEADT |
| 121 | LRKVLVEVLADPLDHRDAGDLWFPGESESFEDAHVEHSISRSLLEGEIPFPPTSILLLLA |
| 181 | CIFLIKILAASALWAAAWHGQKPGTHPPSELDCGHDPGYQLQTLPGLRDT |

Amino acids 1 to 18 of the wild-type human TREM2 protein (SEQ ID NO: 1) is a signal peptide, which is generally removed from the mature protein. The mature human TREM2 protein comprises an extracellular domain at amino acids 19-174 of SEQ ID NO:1, a transmembrane domain at amino acids 175-195 of SEQ ID NO: 1, and a cytoplasmic domain at amino acids 196-230 of SEQ ID NO: 1. The amino acid sequence of the extracellular domain (including the signal peptide) of human TREM2 is provided below as SEQ ID NO:2.

| (SEQ ID NO: 2) | |
|---|---|
| 1 | MEPLRLLILLFVTELSGAHNTTVFQGVAGQSLQVSCPYDSMKHWGRRKAWCRQLGEKGPC |
| 61 | QRWSTHNLWLLSFLRRWNGSTAITDDTLGGTLTITLRNLQPHDAGLYQCQSLHGSEADT |
| 121 | LRKVLVEVLADPLDHRDAGDLWFPGESESFEDAHVEHSISRSLLEGEIPFPPTS |

The term “human triggering receptor expressed on myeloid cells-2” or “human TREM2” can refer to a polypeptide of SEQ ID NO: 1, a polypeptide of SEQ ID NO:2, polypeptides of SEQ ID NO:1 or SEQ ID NO:2 minus the signal peptide (amino acids 1-18), allelic variants of human TREM2, or splice variants of human TREM2. In some embodiments, the term “human TREM2” includes naturally occurring variants of TREM2, such as mutations R47H, Q33X (X is a stop codon), Y38C, T66M, D87N, H157Y, R98W, and S116C.

Because the cytoplasmic domain of TREM2 lacks signaling capability, it must interact with other proteins to transduce TREM2-activating signals. One such protein is DNAX-activating protein of 12 kDa (DAP 12). DAP12 is also known as killer cell activating receptor-associated protein (KARAP) and tyrosine kinases binding protein (TYROBP). DAP 12 is a type I transmembrane adaptor protein that comprises an ITAM motif in its cytoplasmic domain. The ITAM motif mediates signal propagation by activation of the ZAP70 and Syk tyrosine kinases, which in turn activate several downstream signaling cascades, including PI3K, PKC, ERK, and elevation of intracellular calcium (Colonna, Nature Reviews Immunology, Vol. 3: 445-453, 2003; Ulrich and Holtzman, ACS Che Neurosci., Vol. 7: 420-427, 2016). DAP12 and TREM2 associate through their transmembrane domains; a charged lysine residue within the transmembrane domain of TREM2 interacts with a charged aspartic acid residue within the transmembrane domain of DAP12.

Human DAP12 is encoded by the TYROBP gene located on chromosome 19q13.1. The human protein is 113 amino acids in length and comprises a leader sequence (amino acids 1-27 of SEQ ID NO:3), a short extracellular domain (amino acids 28-41 of SEQ ID NO:3), a transmembrane domain (amino acids 42-65 of SEQ ID NO:3) and a cytoplasmic domain (amino acids 66-113 of SEQ ID NO:3)(Paradowska-Gorycka et al, Human Immunology, Vol. 74: 730-737, 2013). DAP12 forms a homodimer through two cysteine residues in the short extracellular domain. The wild-type human DAP12 amino acid sequence (NCBI Reference Sequence: NP_003323.1) is provided below as SEQ ID NO:3.

| (SEQ ID NO: 3) | |
|---|---|
| 1 | MGGLEPCSRLLLLPLLLAVSGLRPVQAQAQSDCSCSTVSPGVLAGIVMGDLVLTVLIALA |
| 61 | VYFLGRLVPRGRGAAEAATRKQRITETESPYQELQGQRSDVYSDLNTQRPYY |

The term “human DAP 12” can refer to a polypeptide of SEQ ID NO:3, a polypeptide of SEQ ID NO:3 minus the leader peptide (amino acids 1-27), allelic variants of human DAP 12, or splice variants of human DAP 12.

### 6.6. Treatment Methods of the Invention

In one aspect, the present invention provides a method of treating a disease or disorder caused by and/or associated with a TREM2 dysfunction in a human patient, the method comprising administering to the patient a molecule that increases activity of TREM2. In some embodiments, the molecule that increases activity of TREM2 is an agonist of TREM2. In some embodiments, the agonist of TREM2 is an anti-hTREM2 antibody, or an antigen binding-fragment thereof. In some embodiments, the agonist of TREM2 is a small molecule. In some embodiments, the molecule that increases activity of TREM2 is a molecule that prevents the degradation of TREM2. In some embodiments, the molecule that increases activity of TREM2 is an anti-hTREM2 antibody, or an antigen binding-fragment thereof. In some embodiments, the molecule that increases activity of TREM2 is a small molecule.

In some embodiments, administration of the agonist of TREM2 activates DAP12 signaling pathways in the patient, resulting in an increase in microglia proliferation, microglia survival and/or microglia phagocytosis. In some embodiments, administration of the agonist of TREM2 results in a slowing of disease progression.

In some embodiments, the agonist of TREM2 activates TREM2/DAP12 signaling in myeloid cells, including monocytes, dendritic cells, microglial cells and/or macrophages. In some embodiments, an agonist of TREM2 activates, induces, promotes, stimulates, or otherwise increases one or more TREM2 activities. TREM2 activities that are activated or increased by the agonist, include but are not limited to: TREM2 binding to DAP12; DAP12 binding to TREM2; TREM2 phosphorylation, DAP12 phosphorylation; PI3K activation; increased levels of soluble TREM2 (sTREM2); increased levels of soluble CSF1R (sCSF1R); increased expression of one or more anti-inflammatory mediators (e.g., cytokines) selected from the group consisting of IL-12p70, IL-6, and IL-10; reduced expression of one or more pro-inflammatory mediators selected from the group consisting of IFN-a4, IFN-b, IL-6, IL-12 p70, IL-10, TNF, TNF-α, IL-10, IL-8, CRP, TGF-beta members of the chemokine protein families, IL-20 family members, IL-33, LIF, IFN-gamma, OSM, CNTF, TGF-beta, GM-CSF, IL-11, IL-12, IL-17, IL-18, and CRP; increased expression of one or more chemokines selected from the group consisting of CCL2, CCL4, CXCL10, CXCL2, CST7; reduced expression of TNF-α, IL-6, or both; extracellular signal-regulated kinase (ERK) phosphorylation; increased expression of C—C chemokine receptor 7 (CCR7); induction of microglial cell chemotaxis toward CCL19 and CCL21 expressing cells; an increase, normalization, or both of the ability of bone marrow-derived dendritic cells to induce antigen-specific T-cell proliferation; induction of osteoclast production, increased rate of osteoclastogenesis, or both; increasing the survival and/or function of one or more of dendritic cells, macrophages, microglial cells, M1 macrophages and/or microglial cells, activated M1 macrophages and/or microglial cells, M2 macrophages and/or microglial cells, monocytes, osteoclasts, Langerhans cells of skin, and Kupffer cells; induction of one or more types of clearance selected from the group consisting of apoptotic neuron clearance, nerve tissue debris clearance, non-nerve tissue debris clearance, bacteria or other foreign body clearance, disease-causing protein clearance, disease-causing peptide clearance, and disease-causing nucleic acid clearance; induction of phagocytosis of one or more of apoptotic neurons, nerve tissue debris, non-nerve tissue debris, bacteria, other foreign bodies, disease-causing proteins, disease-causing peptides, or disease-causing nucleic acids; normalization of disrupted TREM2/DAP12-dependent gene expression; recruitment of Syk, ZAP70, or both to the TREM2/DAP12 complex; Syk phosphorylation; increased expression of CD83 and/or CD86 on dendritic cells, macrophages, monocytes, and/or microglia; reduced secretion of one or more inflammatory cytokines selected from the group consisting of TNF-α, IL-10, IL-6, MCP-1, IFN-a4, IFN-b, IL-1β, IL-8, CRP, TGF-beta members of the chemokine protein families, IL-20 family members, IL-33, LIF, IFN-gamma, OSM, CNTF, TGF-beta, GM-CSF, IL-11, IL-12, IL-17, IL-18, and CRP; reduced expression of one or more inflammatory receptors; increasing phagocytosis by macrophages, dendritic cells, monocytes, and/or microglia under conditions of reduced levels of MCSF; decreasing phagocytosis by macrophages, dendritic cells, monocytes, and/or microglia in the presence of normal levels of MCSF; increasing activity of one or more TREM2-dependent genes; or any combination thereof.

In another aspect, the invention provides a TREM2 agonist for the manufacture of a medicament for the treatment of a disease or disorder caused by and/or associated with a TREM2 dysfunction.

### 6.7. Diseases and Disorders

Certain aspects of the present invention provide a TREM2 agonist for use in treating, preventing, or ameliorating the risk of developing conditions associated with TREM2 deficiency in a patient in need thereof. In particular embodiments, the use comprises administering to the patient an effective amount of a TREM2 agonist.

Conditions or disorders associated with TREM2 deficiency or loss of TREM2 function that may be prevented, treated, or ameliorated according to the methods of the invention include, but are not limited to, Nasu-Hakola disease, Alzheimer's disease, frontotemporal dementia, multiple sclerosis, Guillain-Barre syndrome, amyotrophic lateral sclerosis, Parkinson's disease, traumatic brain injury, spinal cord injury, systemic lupus erythematosus, rheumatoid arthritis, prion disease, stroke, osteoporosis, osteopetrosis, and osteosclerosis. In certain embodiments, the condition or disorder to be prevented, treated, or ameliorated according to the methods of the invention is Alzheimer's disease, Nasu-Hakola disease, frontotemporal dementia, multiple sclerosis, prion disease, or stroke.

In some embodiments, the condition to be treated, prevented, or ameliorated is Alzheimer's disease. In some embodiments, the patient to be administered a TREM2 agonist antigen binding protein is a patient at risk of developing Alzheimer's disease. The patient in need of treatment may be determined to have one or more genotypes associated with an increased risk of developing a disease or condition that can be treated according to the methods of the invention. For instance, in some embodiments, the patient has a genotype associated with an increased risk of developing Alzheimer's disease, such as the genotypes described herein. In further embodiments, the patient may be determined to carry an allele encoding a TREM2 variant associated with an increased risk of developing Alzheimer's disease. For instance, in one embodiment, the patient has been determined to have at least one allele containing the rs75932628-T mutation in the TREM2 gene, e.g. the patient has a genotype of CT at rs75932628. In related embodiments, the patient having or at risk of developing Alzheimer's disease is a patient who has been determined to carry a TREM2 variant allele that encodes a histidine in place of arginine at position 47 in SEQ ID NO:1 (R47H TREM2 variant). In other embodiments, the patient has been determined to have at least one allele containing the rs143332484-T mutation in the TREM2 gene, e.g. the patient has a genotype of CT at rs143332484. In related embodiments, the patient having or at risk of developing Alzheimer's disease is a patient who has been determined to carry a TREM2 variant allele that encodes a histidine in place of arginine at position 62 in SEQ ID NO:1 (R62H TREM2 variant). In some embodiments, a patient at risk of developing Alzheimer's disease has been determined to have at least one allele containing the rs6910730-G mutation in the TREM1 gene, at least one allele containing the rs7759295-C mutation upstream of the TREM2 gene, and/or at least one E4 allele of the APOE gene.

In another embodiment, the present invention provides a method for preventing, treating, or ameliorating frontotemporal dementia or Nasu-Hakola disease in a patient in need thereof comprising administering to the patient an effective amount of a TREM2 agonist antigen binding protein described herein. In some embodiments, the patient to be administered a TREM2 agonist antigen binding protein is a patient at risk of developing frontotemporal dementia or Nasu-Hakola disease. For example, in one such embodiment, the patient has been determined to have at least one allele containing the rs104894002-A mutation in the TREM2 gene, e.g. the patient has a genotype of GA or AA at rs 104894002. In related embodiments, the patient at risk of developing frontotemporal dementia or Nasu-Hakola disease is a patient who has been determined to carry a TREM2 variant allele that encodes a truncated TREM2 protein as a result of the substitution of a stop codon in place of glutamine at position 33 in SEQ ID NO: 1. In another embodiment, the patient has been determined to have at least one allele containing the rs201258663-A mutation in the TREM2 gene, e.g. the patient has a genotype of GA or AA at rs201258663. In related embodiments, the patient at risk of developing frontotemporal dementia or Nasu-Hakola disease is a patient who has been determined to have any TREM2 variant allele that encodes a methionine in place of threonine at position 66 in SEQ ID NO: 1. In some embodiments, the patient at risk of developing frontotemporal dementia or Nasu-Hakola disease is a patient who has been determined to carry a TREM2 variant allele that encodes a cysteine in place of tyrosine at position 38 in SEQ ID NO: 1.

In yet another embodiment, the present invention provides a method for preventing, treating, or ameliorating multiple sclerosis in a patient in need thereof comprising administering to the patient an effective amount of a TREM2 agonist described herein. In some embodiments, the patient to be administered a TREM2 agonist is a patient at risk of developing multiple sclerosis.

The present invention also includes methods of increasing survival or proliferation of myeloid cells, such as macrophages, microglia, and dendritic cells, in a patient in need thereof. In some embodiments, TREM2 agonist described herein can be used to activate TREM2/DAP12 signaling in myeloid cells, thereby modulating the biological activity of these cells. Such biological activities include cytokine release, phagocytosis, and microgliosis.

The TREM2 agonists described herein can be used in the manufacture of a pharmaceutical composition or medicament for the treatment or prevention of conditions associated with TREM2 deficiency or loss of TREM2 biological activity as described herein, including, inter alia, Alzheimer's disease, Nasu-Hakola disease, frontotemporal dementia, multiple sclerosis, prion disease, or stroke. Thus, the present invention also provides a pharmaceutical composition comprising a TREM2 agonist antigen binding protein described herein and a pharmaceutically acceptable excipient.

In one embodiment, the present invention provides a method for preventing, treating, or ameliorating Alzheimer's disease in a patient in need thereof comprising administering to the patient an effective amount of a TREM2 agonist antigen binding protein described herein. In certain embodiments, the TREM2 agonist administered to the patient is an anti-hTREM2 monoclonal antibody, such as the antibodies whose CDR sequences, variable region sequences, and heavy and light chain sequences are set forth in TABLES 2A-2B and 3.

### 6.8. Target Agonists

The present invention provides for methods of treating, preventing, or ameliorating the risk of developing conditions associated with TREM2 deficiency in a patient in need thereof, the method comprising administering to the patient an effective amount of a molecule that specifically binds to hTREM2, which increases the activity of hTREM2. In some embodiments, the molecule is an agonist of TREM2. In some embodiments, the agonist of TREM2 is a small molecule. In some embodiments, the agonist of TREM2 is an antibody, or antigen-binding fragment thereof.

The TREM2 agonist specifically bind to human TREM2 (SEQ ID NO: 1) or an extra cellular domain (ECD) of human TREM2 (e.g. ECD set forth in SEQ ID NO:2), for example with an equilibrium dissociation constant (K_{D}) less than 50 nM, less than 25 nM, less than 10 nM, or less than 5 nM.

6.8.1. Anti-hTREM2 Antibodies

Antibodies

In one aspect, the invention relates to administration of anti-hTREM2 antibodies, or antigen-binding fragments thereof. While certain embodiments are provided for in the context of intact antibodies, it is contemplated that molecules derived from the antigen-binding fragment of said antibodies may maintain binding specificity and can also be used in the present invention.

In some embodiments, the anti-hTREM2 antibodies are agonists of hTREM2. In some embodiments, the anti-hTREM2 antibodies do not cross-react with other TREM proteins, such as human TREM1 (hTREM1). In some embodiments, the anti-hTREM2 antibodies do not bind to hTREM1, or an isoform or truncation thereof. The amino acid sequence of precursor hTREM1 isoform I (NCBI Reference Sequence: NP_061113.1) is provided below as SEQ ID NO:4.

| (SEQ ID NO: 4) | |
|---|---|
| 1 | MRKTRLWGLLWMLFVSELRAATKLTEEKYELKEGQTLDVKCDYTLEKFASSQKAWQIIRD |
| 61 | GEMPKTLACTERPSKNSHPVQVGRIILEDYHDHGLLRVRMVNLQVEDSGLYQCVIYQPPK |
| 121 | EPHMLFDRIRLVVTKGFSGTPGSNENSTQNVYKIPPTTTKALCPLYTSPRTVTQAPPKST |
| 181 | ADVSTPDSEINLTNVTDIIRVPVFNIVILLAGGFLSKSLVFSVLFAVTLRSFVP |

In some embodiments, the anti-hTREM2 antibodies specifically bind to human TREM2 hTREM2 residues 19-174. In some embodiments, the anti-hTREM2 antibodies specifically bind to IgV region of hTREM2, for example human TREM2 residues 19-140.

In certain embodiments, anti-hTREM2 antibodies of the present disclosure bind to one or more amino acids within amino acid residues 29-112 of hTREM 2 (SEQ ID NO: 1), or within amino acid residues on a TREM2 protein corresponding to amino acid residues 29-112 of SEQ ID NO: 1. In some embodiments, anti-hTREM2 antibodies of the present disclosure bind to one or more amino acids within amino acid residues 29-41 of hTREM 2 (SEQ ID NO: 1), or within amino acid residues on a TREM2 protein corresponding to amino acid residues 29-41 of SEQ ID NO: 1. In some embodiments, anti-hTREM2 antibodies of the present disclosure bind to one or more amino acids within amino acid residues 47-69 of hTREM 2 (SEQ ID NO: 1), or within amino acid residues on a TREM2 protein corresponding to amino acid residues 47-69 of SEQ ID NO: 1. In some embodiments, anti-hTREM2 antibodies of the present disclosure bind to one or more amino acids within amino acid residues 76-86 of hTREM 2 (SEQ ID NO: 1), or within amino acid residues on a TREM2 protein corresponding to amino acid residues 76-86 of SEQ ID NO: 1. In some embodiments, anti-hTREM2 antibodies of the present disclosure bind to one or more amino acids within amino acid residues 91-100 of hTREM2 (SEQ ID NO: 1), or within amino acid residues on a TREM2 protein corresponding to amino acid residues 91-100 of SEQ ID NO: 1. In some embodiments, anti-hTREM2 antibodies of the present disclosure bind to one or more amino acids within amino acid residues 99-115 of hTREM 2 (SEQ ID NO: 1), or within amino acid residues on a TREM2 protein corresponding to amino acid residues 99-115 of SEQ ID NO: 1. In some embodiments, anti-hTREM2 antibodies of the present disclosure bind to one or more amino acids within amino acid residues 104-112 of hTREM 2 (SEQ ID NO: 1), or within amino acid residues on a TREM2 protein corresponding to amino acid residues 104-112 of SEQ ID NO: 1. In some embodiments, anti-hTREM2 antibodies of the present disclosure bind to one or more amino acids within amino acid residues 114-118 of hTREM 2 (SEQ ID NO: 1), or within amino acid residues on a TREM2 protein corresponding to amino acid residues 114-118 of SEQ ID NO: 1. In some embodiments, anti-hTREM2 antibodies of the present disclosure bind to one or more amino acids within amino acid residues 130-171 of hTREM 2 (SEQ ID NO: 1), or within amino acid residues on a TREM2 protein corresponding to amino acid residues 130-171 of SEQ ID NO: 1. In some embodiments, anti-hTREM2 antibodies of the present disclosure bind to one or more amino acids within amino acid residues 139-153 of hTREM 2 (SEQ ID NO: 1), or within amino acid residues on a TREM2 protein corresponding to amino acid residues 139-153 of SEQ ID NO: 1. In some embodiments, anti-hTREM2 antibodies of the present disclosure bind to one or more amino acids within amino acid residues 139-146 of hTREM 2 (SEQ ID NO: 1), or within amino acid residues on a TREM2 protein corresponding to amino acid residues 139-146 of SEQ ID NO: 1. In some embodiments, anti-hTREM2 antibodies of the present disclosure bind to one or more amino acids within amino acid residues 130-144 of hTREM 2 (SEQ ID NO: 1), or within amino acid residues on a TREM2 protein corresponding to amino acid residues 130-144 of SEQ ID NO: 1. In some embodiments, anti-hTREM2 antibodies of the present disclosure bind to one or more amino acids within amino acid residues 158-171 of hTREM 2 (SEQ ID NO: 1), or within amino acid residues on a TREM2 protein corresponding to amino acid residues 158-171 of SEQ ID NO: 1.

In some embodiments, anti-hTREM2 antibodies of the present disclosure bind to one or more amino acids within amino acid residues 43-50 of hTREM 2 (SEQ ID NO: 1), or within amino acid residues on a TREM2 protein corresponding to amino acid residues 43-50 of SEQ ID NO: 1. In some embodiments, anti-hTREM2 antibodies of the present disclosure bind to one or more amino acids within amino acid residues 49-57 of hTREM 2 (SEQ ID NO: 1), or within amino acid residues on a TREM2 protein corresponding to amino acid residues 49-57 of SEQ ID NO: 1. In some embodiments, anti-hTREM2 antibodies of the present disclosure bind to one or more amino acids within amino acid residues 139-146 of hTREM 2 (SEQ ID NO: 1), or within amino acid residues on a TREM2 protein corresponding to amino acid residues 139-146 of SEQ ID NO: 1. In some embodiments, anti-hTREM2 antibodies of the present disclosure bind to one or more amino acids within amino acid residues 140-153 of hTREM 2 (SEQ ID NO: 1), or within amino acid residues on a TREM2 protein corresponding to amino acid residues 140-153 of SEQ ID NO: 1. In some embodiments, anti-hTREM2 antibodies specifically bind to the stalk region of human TREM2, for example amino acid residues 145-174 of human TREM2.

In some embodiments, the anti-hTREM2 antibody, or an antigen-binding fragment thereof, specifically prevents the degradation or cleavage of hTREM2.

As used herein, the term “antibody” (Ab) refers to an immunoglobulin molecule that specifically binds to a particular antigen, e.g., hTREM2. In some embodiments, an anti-hTREM2 antibody is suitable for administration to humans.

In some embodiments, the anti-hTREM2 antibody is a polyclonal antibody. In some embodiments, the anti-hTREM2 antibody is a monoclonal antibody. In some embodiments, the anti-hTREM2 antibody is a chimeric antibody. In some embodiments, the anti-hTREM2 antibody is a humanized antibody. In some embodiments, the anti-hTREM2 antibody is a human antibody, particularly a fully human antibody. In some embodiments, the anti-hTREM2 antibody is a bispecific or other multivalent antibody. In some embodiments, the antibody is a single chain antibody.

In some embodiments, the antibodies comprise all or a portion of a constant region of an antibody. In some embodiments, the constant region is a selected from an isotype selected from: IgA (e.g., IgA₁ or IgA₂), IgD, IgE, IgG (e.g., IgG₁, IgG2, IgG₃ or IgG₄), or IgM. In specific embodiments, the anti-hTREM2 antibodies described herein comprise an IgG₁. In some embodiments, the constant region of the IgG₁ comprises a substitution selected from R292C, N297G, V302C, D356E, or L358M (according to EU numbering). In other embodiments, the anti-hTREM2 antibodies comprise an IgG2. In other embodiments, the anti-hTREM2 antibodies comprise an IgG₄. As used herein, the “constant region” of an antibody includes the natural constant region, or any allotypes or natural variants thereof.

The light constant region of an anti-hTREM2 antibody may comprise a lambda (λ) light region or a kappa (κ) light region. The a light region can be any one of the known subtypes, e.g., λ₁, λ₂, λ₃, or λ₄

The term “monoclonal antibody” as used herein is not limited to antibodies produced through hybridoma technology. A monoclonal antibody is derived from a single clone, including any eukaryotic, prokaryotic, or phage clone, by any means available or known in the art. Monoclonal antibodies useful with the present disclosure can be prepared using a wide variety of techniques known in the art including the use of hybridoma, recombinant, and phage display technologies, or a combination thereof.

The term “chimeric” antibody as used herein refers to an antibody having variable sequences derived from an immunoglobulin of one species, such as a rat or a mouse antibody, and an immunoglobulin constant region of another species, such as a human immunoglobulin template.

Other examples of a chimeric antibody include a human derived immunoglobulin variable region with a murine immunoglobulin constant region.

“Humanized” forms of non-human (e.g., murine) antibodies comprise substantially all of the CDR regions and variable regions of a non-human immunoglobulin and all or substantially all of the FR regions of a human immunoglobulin sequence. The humanized antibody may also comprise at least a portion of an immunoglobulin constant region (Fc).

“Human antibodies” include antibodies having the amino acid sequence of a human immunoglobulin. Human antibodies can be from animals that are transgenic for one or more human immunoglobulins. For example, transgenic animals may lack endogenous production of one or more immunoglobulins, such as the Xenomouse®, and be engineered to produce antibodies with fully human protein sequences upon immunization. Human antibodies can also be made by a variety of methods known in the art, including isolation from human immunoglobulin libraries, or phage display methods using antibody libraries derived from human immunoglobulin sequences.

Anti-hTREM2 antibodies of the disclosure include full-length (intact) antibody molecules, or portions thereof. The anti-hTREM2 antibodies may be antibodies whose sequences have been modified to alter at least one constant region-mediated biological effector function (e.g., improved or reduced binding to one or more of the Fc receptors (FcγR) such as FcγRI, FcγRIIA, FcγRIIB, FcγRIIIA and/or FcγRIIIB).

Anti-hTREM2 antibodies with high affinity for hTREM2 may be desirable for therapeutic and diagnostic uses. Accordingly, the present disclosure contemplates antibodies having a high binding affinity to hTREM2. In specific embodiments, the anti-hTREM2 antibodies binds to hTREM2 with an affinity of at least about 100 nM, but may exhibit higher affinity, for example, at least about 90 nM, 80 nM, 70 nM, 60 nM, 50 nM, 40 nM, 30 nM, 25 nM, 20 nM, 15 nM, 10 nM, 7 nM, 6 nM, 5 nM, 4 nM, 3 nM, 2 nM, 1 nM, 0.1 nM, 0.01 nM, or even higher. In some embodiments, the antibodies bind hTREM2 with an affinity in the range of about 1 pM to about 10 nM, of about 100 pM to about 10 nM, about 100 pM to about 1 nM, or an affinity ranging between any of the foregoing values.

Affinity of anti-hTREM2 antibodies for hTREM2 can be determined using techniques well known in the art or described herein, such as for example, but not by way of limitation, ELISA, isothermal titration calorimetry, surface plasmon resonance, biolayer inferotometry, filter binding, or fluorescent polarization.

Anti-hTREM2 antibodies of the disclosure comprise complementarity determining regions (CDRs) in both the light chain and the heavy chain variable domains. Anti-hTREM2 antibodies comprise a light chain variable region comprising complementarity determining regions CDRL1, CDRL2, and CDRL3 and a heavy chain variable region comprising complementarity determining regions CDRH1, CDRH2, and CDRH3 described herein.

In some embodiments, the TREM2 agonist antigen binding protein comprises a CDRL1 or a variant thereof having one, two, three or four amino acid substitutions; a CDRL2, or a variant thereof having one, two, three or four amino acid substitutions; a CDRL3, or a variant thereof having one, two, three or four amino acid substitutions; a CDRH1, or a variant thereof having one, two, three or four amino acid substitutions; a CDRH2, or a variant thereof having one, two, three or four amino acid substitutions; and a CDRH3, or a variant thereof having one, two, three or four amino acid substitutions, where the amino acid sequences of the CDRL1, CDRL2, CDRL3, CDRH1, CDRH2, and CDRH3 are provided in TABLES 1A and 1B below, along with exemplary light chain and variable regions.

TABLE 1A
Exemplary Anti-hTREM2 Antibody Light Chain Variable Regions

| VL Amino Acid Sequence | CDRL1 | CDRL2 | CDRL3 |
|---|---|---|---|
| EIVMTQSPATLSVSPGERATLSCRASQSVSS | RASQSVSSNLA | GASTRAT | LQDNNFPPT |
| NLAWFQQKPGQAPRLLIYGASTRATGIPARF | SEQ ID NO:6 | SEQ ID NO:7 | SEQ ID NO:8 |
| SGSGSGTEFTLTISSLQPEDFAVYYCLQDNN | | | |
| FPPTFGQGTKVDIK | | | |
| SEQ ID NO: 5 | | | |

TABLE 1B
Exemplary Anti-hTREM2 Antibody Heavy Chain Variable Regions

| VH Amino Acid Sequence | CDRH1 | CDRH2 | CDRH3 |
|---|---|---|---|
| EVQLVQSGAEVKKPGESLKISCKGSGY | SYWIG | IIYPGDADARYSPSFQG | RRQGIFGDALDF |
| SFTSYWIGWVRQMPGKGLEWMGIIYPG | SEQ ID NO: 10 | SEQ ID NO: 11 | SEQ ID NO: 12 |
| DADARYSPSFQGQVTISADKSISTAYL | | | |
| QWSSLKASDTAMYFCARRRQGIFGDAL | | | |
| DFWGQGTLVTVSS | | | |
| SEQ ID NO: 9 | | | |

As noted above, anti-hTREM2 antibodies may comprise one or more of the CDRs presented in TABLE 1A (light chain CDRs; i.e. CDRLs) and TABLE 1B (heavy chain CDRs, i.e. CDRHs).

In some embodiments, an anti-hTREM2 antibody comprises a light chain comprising a CDRL1 having an amino acid sequence according to SEQ ID NO:6, a CDRL2 having an amino acid sequence according to SEQ ID NO:7, a CDRL3 having an amino acid sequence according to SEQ ID NO:8, or any CDRL1, CDRL2, or CDRL3 amino acid sequence that contains one or more, e.g., one, two, three, four or more amino acid substitutions (e.g., conservative amino acid substitutions), deletions or insertions of no more than five, four, three, two, or one amino acids to any of SEQ ID NOS:6-8. Such substitutions, deletions, and insertions would retain significant anti-hTREM2 binding activity In these and other embodiments, an anti-hTREM2 antibody comprises a CDRH1 having an amino acid sequence according to SEQ ID NO: 10, a CDRH2 having an amino acid sequence according to SEQ ID NO: 11, a CDRH3 having an amino acid sequence according to SEQ ID NO: 12, or any CDRH1, CDRH2, or CDRH3 having an amino acid sequence that contains one or more, e.g., one, two, three, four or more amino acid substitutions (e.g., conservative amino acid substitutions), deletions or insertions of no more than five, four, three, two, or one amino acids to any of SEQ ID NOS: 10-12. Such substitutions, deletions, and insertions would retain significant anti-hTREM2 binding activity

In some embodiments, an anti-hTREM2 antibody comprises a light chain variable region comprising a CDRL1 having an amino acid sequence according to SEQ ID NO:6; a CDRL2 having an amino acid sequence according to SEQ ID NO:7; and a CDRL3 having an amino acid sequence according to SEQ ID NO:8, and a heavy chain variable region comprising a CDRH1 having an amino acid sequence according to SEQ ID NO: 10; a CDRH2 having an amino acid sequence according to SEQ ID NO: 11; and a CDRH3 having an amino acid sequence according to SEQ ID NO:12.

In some embodiments, an anti-hTREM2 antibody comprises a light chain variable region having an amino acid sequence according to SEQ ID NO:5, or any amino acid sequence that contains one or more, e.g., one, two, three, four or more amino acid substitutions (e.g., conservative amino acid substitutions), deletions or insertions of no more than five, four, three, two, or one amino acids to SEQ ID NO:5. Such substitutions, deletions, and insertions would retain significant anti-hTREM2 binding activity. In some embodiments, an anti-hTREM2 antibody comprises a heavy chain variable region having an amino acid sequence according to SEQ ID NO:9, or any amino acid sequence that contains one or more, e.g., one, two, three, four or more amino acid substitutions (e.g., conservative amino acid substitutions), deletions or insertions of no more than five, four, three, two, or one amino acids to SEQ ID NO:9. Such substitutions, deletions, and insertions would retain significant anti-hTREM2 binding activity.

In specific embodiments, an anti-hTREM2 antibody comprises a light chain variable region having an amino acid sequence according to SEQ ID NO:5, and a heavy chain variable region having an amino acid sequence according to SEQ ID NO: 9.

In some embodiments, an anti-hTREM2 antibody comprises a heavy chain amino acid sequence, and/or a light chain amino acid sequence selected from TABLE 2.

TABLE 2
Exemplary Anti-hTREM2 Antibody Heavy and Light Chains

| Chain | Description | Amino Acid Sequence |
|---|---|---|
| Light | hT2AB | EIVMTQSPATLSVSPGERATLSCRASQSVSSNLAWFQQKPGQAPRLLIYGASTRAT |
| Chain | | GIPARFSGSGSGTEFTLTISSLQPEDFAVYYCLQDNNFPPTFGQGTKVDIKRTVAA |
| | | PSVFIFPPSDEQLKSGTASVVCLLNNFYPREAKVQWKVDNALQSGNSQESVTEQDS |
| | | KDSTYSLSSTLTLSKADYEKHKVYACEVTHQGLSSPVTKSFNRGEC |
| | | (SEQ ID NO: 13) |
| | hT2AB w/ | MDMRVPAQLLGLLLLWLRGARCEIVMTQSPATLSVSPGERATLSCRASQSVSSNLA |
| | leader | WFQQKPGQAPRLLIYGASTRATGIPARFSGSGSGTEFTLTISSLQPEDFAVYYCLQ |
| | sequence | DNNFPPTFGQGTKVDIKRTVAAPSVFIFPPSDEQLKSGTASVVCLLNNFYPREAKV |
| | | QWKVDNALQSGNSQESVTEQDSKDSTYSLSSTLTLSKADYEKHKVYACEVTHQGLS |
| | | SPVTKSFNRGEC |
| | | (SEQ ID NO: 14) |
| | mT2Ab | EIVMTQSPATLSVSPGERATLSCRASQSVSSNLAWFQQKPGQAPRLLIYGASTRAT |
| | (mIgG1) | GIPARFSGSGSGTEFTLTISSLQPEDFAVYYCLQDNNFPPTFGQGTKVDIKRADAA |
| | | PTVSIFPPSSEQLTSGGASVVCFLNNFYPKDINVKWKIDGSERQNGVLNSWTDQDS |
| | | KDSTYSMSSTLTLTKDEYERHNSYTCEATHKTSTSPIVKSFNRNEC |
| | | (SEQ ID NO: 15) |
| Heavy | hT2AB | EVQLVQSGAEVKKPGESLKISCKGSGYSFTSYWIGWVRQMPGKGLEWMGIIYPGDA |
| Chain | | DARYSPSFQGQVTISADKSISTAYLQWSSLKASDTAMYFCARRRQGIFGDALDFWG |
| | | QGTLVTVSSASTKGPSVFPLAPSSKSTSGGTAALGCLVKDYFPEPVTVSWNSGALT |
| | | SGVHTFPAVLQSSGLYSLSSVVTVPSSSLGTQTYICNVNHKPSNTKVDKKVEPKSC |
| | | DKTHTCPPCPAPELLGGPSVFLFPPKPKDTLMISRTPEVTCVVVDVSHEDPEVKFN |
| | | WYVDGVEVHNAKTKPCEEQYGSTYRCVSVLTVLHQDWLNGKEYKCKVSNKALPAPI |
| | | EKTISKAKGQPREPQVYTLPPSREEMTKNQVSLTCLVKGFYPSDIAVEWESNGQPE |
| | | NNYKTTPPVLDSDGSFFLYSKLTVDKSRWQQGNVFSCSVMHEALHNHYTQKSLSLS |
| | | PGK |
| | | (SEQ ID NO: 16) |
| | hT2AB w/ | MDMRVPAQLLGLLLLWLRGARCEVQLVQSGAEVKKPGESLKISCKGSGYSFTSYWI |
| | leader | GWVRQMPGKGLEWMGIIYPGDADARYSPSFQGQVTISADKSISTAYLQWSSLKASD |
| | sequence | TAMYFCARRRQGIFGDALDFWGQGTLVTVSSASTKGPSVFPLAPSSKSTSGGTAAL |
| | | GCLVKDYFPEPVTVSWNSGALTSGVHTFPAVLQSSGLYSLSSVVTVPSSSLGTQTY |
| | | ICNVNHKPSNTKVDKKVEPKSCDKTHTCPPCPAPELLGGPSVFLFPPKPKDTLMIS |
| | | RTPEVTCVVVDVSHEDPEVKFNWYVDGVEVHNAKTKPCEEQYGSTYRCVSVLTVLH |
| | | QDWLNGKEYKCKVSNKALPAPIEKTISKAKGQPREPQVYTLPPSREEMTKNQVSLT |
| | | CLVKGFYPSDIAVEWESNGQPENNYKTTPPVLDSDGSFFLYSKLTVDKSRWQQGNV |
| | | FSCSVMHEALHNHYTQKSLSLSPGK |
| | | (SEQ ID NO: 17) |
| | mT2AB | EVQLVQSGAEVKKPGESLKISCKGSGYSFTSYWIGWVRQMPGKGLEWMGIIYPGDA |
| | (mIgG1) | DARYSPSFQGQVTISADKSISTAYLQWSSLKASDTAMYFCARRRQGIFGDALDFWG |
| | | QGTLVTVSSAKTTPPSVYPLAPGSAAQTNSMVTLGCLVKGYFPEPVTVTWNSGSLS |
| | | SGVHTFPAVLQSDLYTLSSSVTVPSSPRPSETVTCNVAHPASSTKVDKKIVPRDCG |
| | | CKPCICTVPEVSSVFIFPPKPKDVLTITLTPKVTCVVVDISKDDPEVQFSWFVDDV |
| | | EVHTAQTQPREEQFNSTFRSVSELPIMHQDWLNGKEFKCRVNSAAFPAPIEKTISK |
| | | TKGRPKAPQVYTIPPPKEQMAKDKVSLTCMITDFFPEDITVEWQWNGQPAENYKNT |
| | | QPIMNTNGSYFVYSKLNVQKSNWEAGNTFTCSVLHEGLHNHHTEKSLSHSPGK |
| | | (SEQ ID NO: 18) |

In some embodiments, an anti-hTREM2 antibody comprises a light chain having an amino acid sequence according to any one of SEQ ID NOS: 13-15, or any amino acid sequence that contains one or more, e.g., one, two, three, four or more amino acid substitutions (e.g., conservative amino acid substitutions), deletions or insertions of no more than five, four, three, two, or one amino acids to any one of SEQ ID NOS: 13-15. Such substitutions, deletions, and insertions would retain significant anti-hTREM2 binding activity. In these and other embodiments, an anti-hTREM2 antibody comprises a heavy chain having an amino acid sequence according to any one of SEQ ID NOS: 16-18, or any amino acid sequence that contains one or more, e.g., one, two, three, four or more amino acid substitutions (e.g., conservative amino acid substitutions), deletions or insertions of no more than five, four, three, two, or one amino acids to any one of SEQ ID NOS: 16-18. Such substitutions, deletions, and insertions would retain significant anti-hTREM2 binding activity.

In some embodiments, an anti-hTREM2 antibody comprises a light chain having an amino acid sequence according to SEQ ID NO: 13, and/or a heavy chain variable region having an amino acid sequence according to SEQ ID NO: 16. In other embodiments, an anti-hTREM2 antibody comprises a light chain having an amino acid sequence according to SEQ ID NO: 14, and/or a heavy chain variable region having an amino acid sequence according to SEQ ID NO: 17. In other embodiments, an anti-hTREM2 antibody comprises a light chain having an amino acid sequence according to SEQ ID NO: 15, and/or a heavy chain variable region having an amino acid sequence according to SEQ ID NO: 18.

In certain embodiments, the anti-TREM2 antibody is hT2AB, which is an hTREM2 agonist comprising a light chain having an amino acid sequence according to SEQ ID NO: 13, and a heavy chain variable region having an amino acid sequence according to SEQ ID NO: 16. In certain embodiments, the anti-TREM2 antibody is hT2AB having N-terminal leader sequences, comprising a light chain having an amino acid sequence according to SEQ ID NO: 14, and a heavy chain variable region having an amino acid sequence according to SEQ ID NO: 17. In certain embodiments, the anti-TREM2 antibody is mT2AB, which is a chimera of hT2AB variable regions with murine kappa and IgG1 constant regions, comprising a light chain having an amino acid sequence according to SEQ ID NO: 14 or an effectorless variant thereof, and a heavy chain having an amino acid sequence according to SEQ ID NO: 17 or an effectorless variant thereof.

Polynucleotides

In another aspect, the present disclosure provides polynucleotides encoding the antibodies or antigen binding regions of the described herein. In particular, the polynucleotides are isolated polynucleotides. The polynucleotides may be operatively linked to one or more heterologous control sequences that control gene expression to create a recombinant polynucleotide capable of expressing the polypeptide of interest. Expression constructs containing a heterologous polynucleotide encoding the relevant polypeptide or protein can be introduced into appropriate host cells to express the corresponding polypeptide.

As will be appreciated by those in the art, due to the degeneracy of the genetic code, where the same amino acids are encoded by alternative or synonymous codons, an extremely large number of nucleic acids can be made, all of which encode the CDRs, variable regions, and heavy and light chains or other components of the antigen binding proteins described herein. Thus, having identified a particular amino acid sequence, those skilled in the art could make any number of different nucleic acids, by simply modifying the sequence of one or more codons in a way which does not change the amino acid sequence of the encoded protein. In this regard, the present disclosure includes each and every possible variation of polynucleotides that encode the polypeptides disclosed herein.

An “isolated nucleic acid,” which is used interchangeably herein with “isolated polynucleotide,” is a nucleic acid that has been separated from adjacent genetic sequences present in the genome of the organism from which the nucleic acid was isolated, in the case of nucleic acids isolated from naturally-occurring sources. In the case of nucleic acids synthesized enzymatically from a template or chemically, such as PCR products, cDNA molecules, or oligonucleotides for example, it is understood that the nucleic acids resulting from such processes are isolated nucleic acids. An isolated nucleic acid molecule refers to a nucleic acid molecule in the form of a separate fragment or as a component of a larger nucleic acid construct. In one preferred embodiment, the nucleic acids are substantially free from contaminating endogenous material.

In some embodiments, the polynucleotide encodes a CDR L1, CDR L2 and CDR L3 of a light chain variable region described herein. In some embodiments, the polynucleotide encodes a CDR H1, CDR H2 and CDR H3 of a heavy chain variable region described herein.

In some embodiments, the polynucleotide encodes a CDR L1, CDR L2 and CDR L3 of a light chain variable region and a CDR H1, CDR H2 and CDR H3 of a heavy chain variable region described herein.

In some embodiments, the polynucleotide encodes a light chain variable region VL having at least 80%, 85%, 90%, 91%, 92%, 93%, 94%, 95%, 96%, 97%, 98%, 99% or greater sequence identity to the amino acid sequence of a variable light chain disclosed herein.

In some embodiments, the polynucleotide encodes a heavy chain variable region VH having at least 80%, 85%, 90%, 91%, 92%, 93%, 94%, 95%, 96%, 97%, 98%, 99% or greater sequence identity to the amino acid sequence of a variable heavy chain disclosed herein.

In some embodiments, the polynucleotides herein may be manipulated in a variety of ways to provide for expression of the encoded polypeptide. In some embodiments, the polynucleotide is operably linked to control sequences, including among others, transcription promoters, leader sequences, transcription enhancers, ribosome binding or entry sites, termination sequences, and polyadenylation sequences for expression of the polynucleotide and/or corresponding polypeptide. Manipulation of the isolated polynucleotide prior to its insertion into a vector may be desirable or necessary depending on the expression vector. The techniques for modifying polynucleotides and nucleic acid sequences utilizing recombinant DNA methods are well known in the art. Guidance is provided in Sambrook et al., Molecular Cloning: A Laboratory Manual, 3rd Ed., Cold Spring Harbor Laboratory Press (2001); and Current Protocols in Molecular Biology, Ausubel. F. ed., Greene Pub. Associates (1998), updates to 2013.

In some embodiments, variants of the antigen binding proteins, including the variants described herein, can be prepared by site-specific mutagenesis of nucleotides in the DNA encoding the polypeptide, using cassette or PCR mutagenesis or other techniques well known in the art, to produce DNA encoding the variant, and thereafter expressing the recombinant DNA in cell culture as outlined herein. However, antigen binding proteins comprising variant CDRs having up to about 100-150 residues may be prepared by in vitro synthesis using established techniques. The variants typically exhibit the same qualitative biological activity as the naturally occurring analogue, e.g., binding to antigen. Such variants include, for example, deletions and/or insertions and/or substitutions of residues within the amino acid sequences of the antigen binding proteins. Any combination of deletion, insertion, and substitution is made to arrive at the final construct, provided that the final construct possesses the desired characteristics. The amino acid changes also may alter post-translational processes of the antigen binding protein, such as changing the number or position of glycosylation sites. In some embodiments, antigen binding protein variants are prepared with the intent to modify those amino acid residues which are directly involved in epitope binding. In other embodiments, modification of residues which are not directly involved in epitope binding or residues not involved in epitope binding in any way, is desirable, for purposes discussed herein. Mutagenesis within any of the CDR regions, framework regions, and/or constant regions is contemplated. Covariance analysis techniques can be employed by the skilled artisan to design useful modifications in the amino acid sequence of the antigen binding protein. See, e.g., Choulier, et al., Proteins 41:475-484, 2000; Demarest et al., J. Mol. Biol., 2004, 335:41-48; Hugo et al., Protein Engineering, 2003, 16(5):381-86; Aurora et al., US Patent Publication No. 2008/0318207 A1; Glaser et al., US Patent Publication No. 2009/0048122 A1; Urech et al., WO 2008/110348 A1; Borras et al., WO 2009/000099 A2. Such modifications determined by covariance analysis can improve potency, pharmacokinetic, pharmacodynamic, and/or manufacturability characteristics of an antigen binding protein.

In another aspect, the present invention also provides vectors comprising one or more nucleic acids or polynucleotides encoding one or more components of the antigen binding proteins describe herein (e.g. variable regions, light chains, and heavy chains). As used herein, the term “vector” refers to any molecule or entity (e.g., nucleic acid, plasmid, bacteriophage or virus) used to transfer protein coding information into a host cell. Examples of vectors include, but are not limited to, plasmids, viral vectors, non-episomal mammalian vectors and expression vectors, for example, recombinant expression vectors. The term “expression vector” or “expression construct” as used herein refers to a recombinant DNA molecule containing a desired coding sequence and appropriate nucleic acid control sequences necessary for the expression of the operably linked coding sequence in a particular host cell. An expression vector can include, but is not limited to, sequences that affect or control transcription, translation, and, if introns are present, affect RNA splicing of a coding region operably linked thereto. Nucleic acid sequences necessary for expression in prokaryotes include a promoter, optionally an operator sequence, a ribosome binding site and possibly other sequences. Eukaryotic cells are known to utilize promoters, enhancers, and termination and polyadenylation signals. A secretory signal peptide sequence can also, optionally, be encoded by the expression vector, operably linked to the coding sequence of interest, so that the expressed polypeptide can be secreted by the recombinant host cell, for more facile isolation of the polypeptide of interest from the cell, if desired.

The recombinant expression vector may be any vector (e.g., a plasmid or virus), which can be conveniently subjected to recombinant DNA procedures and can bring about the expression of the polynucleotide sequence. The choice of the vector will typically depend on the compatibility of the vector with the host cell into which the vector is to be introduced. The vectors may be linear or closed circular plasmids. Exemplary expression vectors include, among others, vectors based on T7 or T7lac promoters (pACY: Novagen; pET); vectors based on Baculovirus promoters (e.g., pBAC); vectors based on Ef1-α and HTLV promoters (e.g., pFUSE2; Invitrogen, CA, USA); vectors based on CMV enhancer and human ferritin light chain gene promoters (e.g., pFUSE: Invitrogen, CA, USA); vectors based on CMV promoters (e.g, pFLAG: Sigma, USA); and vectors based on dihydrofolate reductase promoters (e.g., pEASE: Amgen, USA). Various vectors can be used for transient or stable expression of the polypeptides of interest.

Host Cells

In another aspect, the polynucleotide encoding the antigen binding proteins described herein (e.g. variable regions, light chains, and heavy chains) is operatively linked to one or more control sequences for expression of the polypeptide in the host cell. Accordingly, in a further aspect, the present disclosure provides a host cell comprising one or more expression vectors encoding the components of the TREM2 agonist antigen binding proteins described herein.

Exemplary host cells include prokaryote, yeast, or higher eukaryote cells. Prokaryotic host cells include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacillus, such as B. subtilis and B. licheniformis, Pseudomonas, and Streptomyces. Eukaryotic microbes such as filamentous fungi or yeast are suitable cloning or expression hosts for recombinant polypeptides. Saccharomyces cerevisiae, or common baker's yeast, is the most commonly used among lower eukaryotic host microorganisms. However, a number of other genera, species, and strains are commonly available and useful herein, such as Pichia, e.g. P. pastoris, Schizosaccharomyces pombe; Kluyveromyces, Yarrowia; Candida; Trichoderma reesia; Neurospora crassa; Schwanniomyces, such as Schwanniomyces occidentalis; and filamentous fungi, such as, e.g., Neurospora, Penicillium, Tolypocladium, and Aspergillus hosts such as A. nidulans and A. niger.

Host cells for the expression of glycosylated antigen binding proteins can be derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection of such cells are publicly available, e.g., the L-1 variant of Autographa californica NPV and the Bm-5 strain of Bombyx mori NPV.

Vertebrate host cells are also suitable hosts, and recombinant production of antigen binding proteins from such cells has become routine procedure. Mammalian cell lines available as hosts for expression are well known in the art and include, but are not limited to, immortalized cell lines available from the American Type Culture Collection (ATCC), including but not limited to Chinese hamster ovary (CHO) cells, including CHOK1 cells (ATCC CCL61), DXB-11, DG-44, and Chinese hamster ovary cells/-DHFR (CHO, Urlaub et al., Proc. Natl. Acad. Sci. USA, 1980, 77: 4216); monkey kidney CV1 line transformed by SV40 (COS-7, ATCC CRL 1651); human embryonic kidney line (293 or 293 cells subcloned for growth in suspension culture, (Graham et al., J. Gen Virol. 36: 59, 1977); baby hamster kidney cells (BHK, ATCC CCL 10); mouse sertoli cells (TM4, Mather, Biol. Reprod., 1980, 23:243-251); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human hepatoma cells (Hep G2, HB 8065); mouse mammary tumor (MMT 060562, ATCC CCL51); TRI cells (Mather et al., Annals N.Y Acad. Sci., 1982, 383:44-68); MRC 5 cells or FS4 cells; mammalian myeloma cells, and a number of other cell lines. In certain embodiments, cell lines may be selected through determining which cell lines have high expression levels and constitutively produce antigen binding proteins with human TREM2 binding properties. In another embodiment, a cell line from the B cell lineage that does not make its own antibody but has a capacity to make and secrete a heterologous antibody can be selected. CHO cells are preferred host cells in some embodiments for expressing the TREM2 agonist antigen binding proteins of the invention.

In various embodiments, introduction and transformation of a host cell with a polynucleotide of the present disclosure, such as an expression vector for expressing an antigen binding protein, is accomplished by methods that including transfection, infection, calcium phosphate co-precipitation, electroporation, microinjection, lipofection, DEAE-dextran mediated transfection, or other known techniques. In some embodiments, the method selected can be guided by the type of host cell used. Suitable methods are described in, for example, Sambrook et al., 2001.

Expression and Isolation

In some embodiments, the host cell comprising a polynucleotide encoding one or more components of the antigen binding proteins described herein (e.g. variable regions, light chains, and heavy chains) is used to express the antigen binding protein of interest. In some embodiments, a method for expressing the antigen binding protein comprises culturing the host cell in suitable media and conditions appropriate for expression of the protein of interest.

The type of media and culture conditions selected is based on the type of host cell. In some embodiments, exemplary media for mammalian host cells include, by way of example and not limitation, Ham's F10 (Sigma), Minimal Essential Medium (MEM, Sigma), RPMI-1640 (Sigma), and Dulbecco's Modified Eagle's Medium (DMEM, Sigma. In some embodiments, the media can be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin™ drug), trace elements (defined as inorganic compounds usually present at final concentrations in the micromolar range), and glucose or an equivalent energy source. In some embodiments, culture conditions, such as temperature, pH, % CO₂, and the like, can use conditions available and known to the skilled artisan.

In some embodiments, the expressed antigen binding protein is isolate and/or purified from the host cell. In some embodiments in which the expressed protein in present in the media, the media containing the expressed protein is subject to isolation procedures. In some embodiments in which the antigen binding protein is produced intracellularly, the cells are subject to disruption, and as a first step, the particulate debris, either host cells or lysed fragments, is removed, for example, by centrifugation or ultrafiltration. Subsequently, the antigen binding protein can be isolated and further purified by various known techniques. Such isolation techniques include affinity chromatography with Protein-A Sepharose, size-exclusion chromatography, ion-exchange chromatography, high performance liquid chromatography, differential solubility, and the like (see, e.g., Fisher, Laboratory Techniques, In Biochemistry And Molecular Biology, Work and Burdon, eds., Elsevier (1980); Antibodies: A Laboratory Manual, Greenfield, E. A., ed., Cold Spring Harbor Laboratory Press, New York (2012); Coligan, et al., supra, sections 2.7.1-2.7.12 and sections 2.9.1-2.9.3; Barnes, et al., Purification of Immunoglobulin G (IgG), in Methods Mol. Biol., Vol. 10, pages 79-104, Humana Press (1992)).

In some embodiments, the isolated antibody can be further purified as measurable by: (1) weight of protein as determined using the Lowry method; (2) to a degree sufficient to obtain at least 15 residues of N-terminal or internal amino acid sequence by use of a spinning-cup sequencer; or (3) to homogeneity by SDS-PAGE under reducing or non-reducing conditions using Coomassie blue or, preferably, silver stain. The purified antibody can be 85% or greater, 90% or greater, 95% or greater, or at least 99% by weight as determined by the foregoing methods.

Antibody Formulations

In certain embodiments, the invention provides a composition (e.g. a pharmaceutical composition) comprising one or a plurality of the TREM2 activating antibodies and TREM2 agonist antibodies and antigen binding proteins disclosed herein together with pharmaceutically acceptable diluents, carriers, excipients, solubilizers, emulsifiers, preservatives, and/or adjuvants. Pharmaceutical compositions of the invention include, but are not limited to, liquid, frozen, and lyophilized compositions. “Pharmaceutically-acceptable” refers to molecules, compounds, and compositions that are non-toxic to human recipients at the dosages and concentrations employed and/or do not produce allergic or adverse reactions when administered to humans. In some embodiments, the pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. Methods and suitable materials for formulating molecules for therapeutic use are known in the pharmaceutical arts, and are described, for example, in Remington's Pharmaceutical Sciences, 18th Ed., (A. R. Genrmo, ed.), 1990, Mack Publishing Company.

In some embodiments, the pharmaceutical composition of the invention comprises a standard pharmaceutical carrier, such as a sterile phosphate buffered saline solution, bacteriostatic water, and the like. A variety of aqueous carriers may be used, e.g., water, buffered water, 0.4% saline, 0.3% glycine and the like, and may include other proteins for enhanced stability, such as albumin, lipoprotein, globulin, etc., subjected to mild chemical modifications or the like.

Exemplary concentrations of the antigen binding proteins in the formulation may range from about 0.1 mg/ml to about 200 mg/ml or from about 0.1 mg/mL to about 50 mg/mL, or from about 0.5 mg/mL to about 25 mg/mL, or alternatively from about 2 mg/mL to about 10 mg/mL. An aqueous formulation of the antigen binding protein may be prepared in a pH-buffered solution, for example, at pH ranging from about 4.5 to about 6.5, or from about 4.8 to about 5.5, or alternatively about 5.0. Examples of buffers that are suitable for a pH within this range include acetate (e.g. sodium acetate), succinate (such as sodium succinate), gluconate, histidine, citrate and other organic acid buffers. The buffer concentration can be from about 1 mM to about 200 mM, or from about 10 mM to about 60 mM, depending, for example, on the buffer and the desired isotonicity of the formulation.

A tonicity agent, which may also stabilize the antigen binding protein, may be included in the formulation. Exemplary tonicity agents include polyols, such as mannitol, sucrose or trehalose. Preferably the aqueous formulation is isotonic, although hypertonic or hypotonic solutions may be suitable. Exemplary concentrations of the polyol in the formulation may range from about 1% to about 15% w/v.

A surfactant may also be added to the antigen binding protein formulation to reduce aggregation of the formulated antigen binding protein and/or minimize the formation of particulates in the formulation and/or reduce adsorption. Exemplary surfactants include nonionic surfactants such as polysorbates (e.g., polysorbate 20 or polysorbate 80) or poloxamers (e.g., poloxamer 188). Exemplary concentrations of surfactant may range from about 0.001% to about 0.5%, or from about 0.005% to about 0.2%, or alternatively from about 0.004% to about 0.01% w/v.

In one embodiment, the formulation contains the above-identified agents (i.e. antigen binding protein, buffer, polyol and surfactant) and is essentially free of one or more preservatives, such as benzyl alcohol, phenol, m-cresol, chlorobutanol and benzethonium chloride. In another embodiment, a preservative may be included in the formulation, e.g., at concentrations ranging from about 0.1% to about 2%, or alternatively from about 0.5% to about 1%. One or more other pharmaceutically acceptable carriers, excipients or stabilizers such as those described in REMINGTON'S PHARMACEUTICAL SCIENCES, 18th Edition, (A. R. Genrmo, ed.), 1990, Mack Publishing Company, may be included in the formulation provided that they do not adversely affect the desired characteristics of the formulation.

Therapeutic formulations of the antigen binding protein are prepared for storage by mixing the antigen binding protein having the desired degree of purity with optional physiologically acceptable carriers, excipients or stabilizers (Remington's Pharmaceutical Sciences, 18th Ed., (A. R. Genrmo, ed.), 1990, Mack Publishing Company), in the form of lyophilized formulations or aqueous solutions. Acceptable carriers, excipients, or stabilizers are nontoxic to recipients at the dosages and concentrations employed, and include buffers (e.g. phosphate, citrate, and other organic acids); antioxidants (e.g. ascorbic acid and methionine); preservatives (such as octadecyldimethylbenzyl ammonium chloride, hexamethonium chloride, benzalkonium chloride, benzethonium chloride, phenol, butyl or benzyl alcohol, alkyl parabens such as methyl or propyl paraben, catechol; resorcinol, cyclohexanol, 3-pentanol, and m-cresol); low molecular weight (e.g. less than about 10 residues) polypeptides; proteins (such as serum albumin, gelatin, or immunoglobulins); hydrophilic polymers (e.g. polyvinylpyrrolidone); amino acids (e.g. glycine, glutamine, asparagine, histidine, arginine, or lysine); monosaccharides, disaccharides, and other carbohydrates including glucose, mannose, maltose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants, such as polysorbates (e.g. polysorbate 20 or polysorbate 80) or poloxamers (e.g. poloxamer 188); or polyethylene glycol (PEG).

In one embodiment, a suitable formulation of the claimed invention contains an isotonic buffer such as a phosphate, acetate, or TRIS buffer in combination with a tonicity agent, such as a polyol, sorbitol, sucrose or sodium chloride, which tonicifies and stabilizes. One example of such a tonicity agent is 5% sorbitol or sucrose. In addition, the formulation could optionally include a surfactant at 0.01% to 0.02% wt/vol, for example, to prevent aggregation or improve stability. The pH of the formulation may range from 4.5 to 6.5 or 4.5 to 5.5. Other exemplary descriptions of pharmaceutical formulations for antigen binding proteins may be found in US Patent Publication No. 2003/0113316 and U.S. Pat. No. 6,171,586, each of which is hereby incorporated by reference in its entirety.

Suspensions and crystal forms of antigen binding proteins are also contemplated. Methods to make suspensions and crystal forms are known to one of skill in the art.

The formulations to be used for in vivo administration must be sterile. The compositions of the invention may be sterilized by conventional, well-known sterilization techniques. For example, sterilization is readily accomplished by filtration through sterile filtration membranes. The resulting solutions may be packaged for use or filtered under aseptic conditions and lyophilized, the lyophilized preparation being combined with a sterile solution prior to administration.

The process of freeze-drying is often employed to stabilize polypeptides for long-term storage, particularly when the polypeptide is relatively unstable in liquid compositions. A lyophilization cycle is usually composed of three steps: freezing, primary drying, and secondary drying (see Williams and Polli, Journal of Parenteral Science and Technology, 1984, 38(2):48-59). In the freezing step, the solution is cooled until it is adequately frozen. Bulk water in the solution forms ice at this stage. The ice sublimes in the primary drying stage, which is conducted by reducing chamber pressure below the vapor pressure of the ice, using a vacuum. Finally, sorbed or bound water is removed at the secondary drying stage under reduced chamber pressure and an elevated shelf temperature. The process produces a material known as a lyophilized cake. Thereafter the cake can be reconstituted prior to use.

The standard reconstitution practice for lyophilized material is to add back a volume of pure water (typically equivalent to the volume removed during lyophilization), although dilute solutions of antibacterial agents are sometimes used in the production of pharmaceuticals for parenteral administration (see Chen, Drug Development and Industrial Pharmacy, Volume 18: 1311-1354, 1992).

Excipients have been noted in some cases to act as stabilizers for freeze-dried products (see Carpenter et al., Volume 74: 225-239, 1991). For example, known excipients include polyols (including mannitol, sorbitol and glycerol); sugars (including glucose and sucrose); and amino acids (including alanine, glycine and glutamic acid).

In addition, polyols and sugars are also often used to protect polypeptides from freezing and drying-induced damage and to enhance the stability during storage in the dried state. In general, sugars, in particular disaccharides, are effective in both the freeze-drying process and during storage. Other classes of molecules, including mono- and di-saccharides and polymers such as PVP, have also been reported as stabilizers of lyophilized products.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

Sustained-release preparations may be prepared. Suitable examples of sustained-release preparations include semipermeable matrices of solid hydrophobic polymers containing the antigen binding protein, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the Lupron Depot™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated polypeptides remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

The formulations of the invention may be designed to be short-acting, fast-releasing, long-acting, or sustained-releasing. Thus, the pharmaceutical formulations may also be formulated for controlled release or for slow release.

Specific dosages may be adjusted depending on the disease, disorder, or condition to be treated, the age, body weight, general health conditions, sex, and diet of the subject, dose intervals, administration routes, excretion rate, and combinations of drugs.

The TREM2 agonist antigen binding proteins of the invention can be administered by any suitable means, including parenteral, subcutaneous, intraperitoneal, intrapulmonary, intrathecal, intracerebral, intracerebroventricular, and intranasal, and, if desired for local treatment, intralesional administration. Parenteral administration includes intravenous, intraarterial, intraperitoneal, intramuscular, intradermal or subcutaneous administration. In addition, the antigen binding protein is suitably administered by pulse infusion, particularly with declining doses of the antigen binding protein. Preferably, the dosing is given by injections, most preferably intravenous or subcutaneous injections, depending in part on whether the administration is brief or chronic. Other administration methods are contemplated, including topical, particularly transdermal, transmucosal, rectal, oral or local administration e.g. through a catheter placed close to the desired site. In certain embodiments, the TREM2 agonist antigen binding protein of the invention is administered intravenously or subcutaneously in a physiological solution at a dose ranging between 0.01 mg/kg to 100 mg/kg at a frequency ranging from daily to weekly to monthly (e.g. every day, every other day, every third day, or 2, 3, 4, 5, or 6 times per week), preferably a dose ranging from 0.1 to 45 mg/kg, 0.1 to 15 mg/kg or 0.1 to 10 mg/kg at a frequency of once per week, once every two weeks, or once a month.

The TREM2 agonist antigen binding proteins described herein (e.g. anti-TREM2 agonist monoclonal antibodies and binding fragments thereof) are useful for preventing, treating, or ameliorating a condition associated with TREM2 deficiency or loss of biological function of TREM2 in a patient in need thereof. As used herein, the term “treating” or “treatment” is an intervention performed with the intention of preventing the development or altering the pathology of a disorder. Accordingly, “treatment” refers to both therapeutic treatment and prophylactic or preventative measures. Patients in need of treatment include those already diagnosed with or suffering from the disorder or condition as well as those in which the disorder or condition is to be prevented, such as patients who are at risk of developing the disorder or condition based on, for example, genetic markers. “Treatment” includes any indicia of success in the amelioration of an injury, pathology or condition, including any objective or subjective parameter such as abatement, remission, diminishing of symptoms, or making the injury, pathology or condition more tolerable to the patient, slowing in the rate of degeneration or decline, making the final point of degeneration less debilitating, or improving a patient's physical or mental well-being. The treatment or amelioration of symptoms can be based on objective or subjective parameters, including the results of a physical examination, self-reporting by a patient, cognitive tests, motor function tests, neuropsychiatric exams, and/or a psychiatric evaluation.

### 7. EXAMPLES

The following Examples, which highlight certain features and properties of the exemplary embodiments of the antibodies and binding fragments described herein are provided for purposes of illustration, and not limitation.

### 7.1. Example 1: Prior Activation State Shapes the Microglia Response to Anti-Human TREM2 Antibody in a Mouse Model of Alzheimer's Disease

### 7.1.1. Abstract

Triggering receptor expressed on myeloid cells 2 (TREM2) sustains microglia response to brain injury stimuli including apoptotic cells, myelin damage, and amyloid β (Aβ). Alzheimer's Disease (AD) risk is associated with the TREM2^{R47H} variant, which impairs ligand binding and consequently microglia responses to Aβ pathology. Here it is shown that TREM2 engagement by the mAb hT2AB as surrogate ligand activates microglia in 5XFAD transgenic mice that accumulate Aβ and express either the common TREM2 variant (TREM12cv) or TREM2^{R47H}. scRNA-seq of microglia from TREM2^{CV}-5XFAD mice treated once with control hIgG1 exposed four distinct trajectories of microglia activation leading to disease-associated (DAM), interferon-responsive (IFN-R), cycling (Cyc-M), and MHC-II expressing (MHC-II) microglia types. All of these were underrepresented in TREM2^{R47H}-5XFAD mice, suggesting that TREM2 ligand engagement is required for microglia activation trajectories. Moreover, Cyc-M and IFN-R microglia were more abundant in female than male TREM2^{CV}-5XFAD mice, likely due to greater Aβ load in female 5XFAD mice. A single systemic injection of hT2AB replenished Cyc-M, IFN-R, and MHC-II pools in TREM2^{R47H}-5XFAD mice. In TREM2^{CV}-5XFAD mice, however, hT2AB brought the representation of male Cyc-M and IFN-R microglia closer to that of females, in which these trajectories had already reached maximum capacity. Moreover, hT2AB induced shifts in gene expression patterns in all microglial pools without affecting representation. Repeated treatment with a murinized hT2AB version over 10 days increased chemokines brain content in TREM2^{R47H}-5XFAD mice, consistent with microglia expansion. Thus, the impact of hT2AB on microglia is shaped by the extent of TREM2 endogenous ligand engagement and basal microglia activation.

### 7.1.2. Significance

Alzheimer's disease (AD) is the most common dementia; no therapy halts its progression. Microglial responses that modulate disease course are triggered by AD pathology, including amyloid-β (Aβ) plaques, neurofibrillary tangles and synapse loss. The TREM2 receptor promotes microglia responses to pathology and a variant, TREM2^{R47H}, impairs ligand-binding and correlates with increased AD risk. Employing scRNA-seq, we asked: can an anti-TREM2 antibody, acting as a surrogate ligand, stimulate microglia in mice that accumulate Aβ and express either the common TREM2 variant (TREM12^{CV}) or TREM2^{R47H}? One systemic injection of anti-TREM2 restored microglia activation in TREM2^{R47H} mice, but promoted limited activation in mice carrying TREM2^{CV}, which binds endogenous ligands. Thus, anti-TREM2 can strengthen microglial responses during AD, contingent on pre-existing TREM2 engagement and basal activation.

### 7.1.3. Introduction

Alzheimer's disease (AD) is the most common cause of progressive dementia in older adults; it affects more than 5.5 million Americans, most 65 years of age or older, and represents the 6^{th} leading cause of death in the United States (https://www.nia.nih.gov/health/alzheimers-disease-fact-sheet). The pathological features of AD include extracellular amyloid plaques composed of the amyloid β (Aβ) peptide, intraneuronal neurofibrillary tangles consisting of aggregated, hyperphosphorylated tau protein, neuroimmune activation, and reductions in synaptic density (1). Longitudinal natural history studies such as the Alzheimer's Disease Neuroimaging Initiative have demonstrated that deposition of Aβ in the central nervous system (CNS) occurs early in disease and is followed by subsequent tau pathology followed by neuronal cell death and cognitive impairment (2). Aβ accumulation also elicits a response by microglia, brain resident macrophages that support development, function and immune defense of the CNS (3).

While all dominant mutations causing familial early-onset AD occur either in the substrate (amyloid precursor protein, APP) or the proteases (presenilins) of the proteolytic pathway that generates Aβ (4), genome-wide association studies found many genetic polymorphisms linked with late-onset AD in genes expressed by microglia, such as TREM2, CD33, SPI1, SHIP1 and PLCγ2 (5, 6), providing strong evidence that microglia actively control the onset and/or progression of AD pathology (7). Microglia accumulate around Aβ plaques to contain and compact them, thereby reducing markers of axonal dystrophy in surrounding neurons (8). During this process, microglia modify their phenotypic and transcriptional properties, transitioning from a “homeostatic” to an activated profile often defined as disease associated microglia (DAM) (6). This transition to the DAM phenotype is robustly activated in transgenic amyloid murine models of AD (9, 10), but is observed to a lesser extent in human AD post-mortem brains (11, 12), potentially reflecting insufficient microglial response to CNS damage in individuals who develop AD pathology.

Microglia transition to DAM has been shown to depend on Triggering Receptor Expressed in Myeloid cells 2 (TREM2), a macrophage cell surface receptor abundantly expressed in microglia (13). TREM2 is a member of the immunoglobulin superfamily that binds phospholipids, apoptotic cells, lipoproteins, such as HDL, LDL, and ApoE, as well as Aβ. TREM2 transmits intracellular signals through the associated adaptor DAP12, which recruits the protein tyrosine kinase Syk, leading to a cascade of protein tyrosine phosphorylation events that promote proliferation, survival, production of ATP and protein biosynthesis. The ectodomain of TREM2 is cleaved from the cell surface by proteases, thereby limiting TREM2 signaling and releasing soluble TREM2 (sTREM2) (14, 15). Genetic variants in TREM2 are associated with multiple neurodegenerative diseases, including Nasu-Hakola disease, fronto-temporal dementia, and AD. Because of its role in metabolic activation, TREM2 may function as a costimulatory molecule that sustains microglia activation during transition to DAM, which is initiated by various receptors engaged by CNS injury stimuli, such as Aβ, apoptotic cell debris and myelin damage (13).

Several observations have suggested that activation of microglia through TREM2 may provide a promising therapeutic approach in AD. First, an arginine-to-histidine missense substitution at amino acid 47 (TREM2^{R47H}) that increases AD risk 2 to 5 fold (16, 17) impairs ligand binding (18) and curtails microglia activation in humans (11) and mouse models of AD (8, 19). Second, complete deletion of Trem2 expression in mice that accumulate Aβ plaques weakens microglial encapsulation of Aβ plaques, which enhances their neurotoxicity (20, 21), and blocks the conversion of microglia from homeostatic to DAM (9). Conversely, mice overexpressing TREM2 evince less Aβ-induced pathology (22). Finally, anti-TREM2 activating antibodies were recently shown to boost microglia responses to Aβ in vitro (23), moderate Aβ plaque load after short-term treatment (24), and promote microglia proliferation as well as attenuate the neurotoxic effects of Aβ plaques after long-term administration (25).

In this study, we characterized the biologic effects in the 5XFAD model of a new anti-human agonistic TREM2 mAb, designated hT2AB, and a murinized version of this agonist TREM2 mAb, designated mT2AB. This antibody binds the common TREM2 variant (TREM2^{CV}) and the AD-associated TREM2^{R47H} variant. hT2AB was tested in transgenic mice that express either TREM2^{CV} or human TREM2^{R47H} in place of endogenous TREM2 (19). These mice were crossed with 5XFAD transgenic mice, which expresses human APP and PSEN1 transgenes with a total of five AD-linked mutations that promote the accumulation of Aβ plaques (26). One feature of this model is a sex bias in amyloid pathology: female 5XFAD mice have more pronounced amyloid pathology than do males (27, 28).

We first showed that hT2AB is a TREM2 agonist which can cross the blood-brain barrier (BBB) after systemic administration. We next examined the effects of a single intraperitoneal injection of hT2AB or control hIgG1 on microglia by single-cell RNA-seq (scRNA-seq). In control hIgG1-treated mice, microglia acquired a continuum of cell-state transitions from homeostatic towards four different types, including DAM, interferon-responsive (IFN-R), cycling (Cyc-M), and MHC-II expressing (MHC-II), which likely reflected engagement of different signaling pathways. All trajectories required TREM2, as indicated by a significant enrichment of terminal microglial types in TREM2^{CV}-5XFAD mice compared to TREM2^{R47H}-5XFAD and Trem2^{-/-}-5XFAD mice. Further, Cyc-M and IFN-R microglia were more abundant in females than males, which correlated with a higher degree of Aβ accumulation in females. hT2AB promoted cell cycle re-entry in TREM2^{R47H}-5XFAD mice and promoted Cyc-M expansion more effectively in males than in females within the TREM2^{CV}-5XFAD cohort. Likewise, hT2AB induced the terminal IFN-R population in TREM2^{R47H}-5XFAD of both sexes, as well as TREM2^{CV}-5XFAD males, but did not promote this cell fate in TREM2^{CV}-5XFAD females, in which this population was already robustly induced. Thus, mAb-mediated engagement of TREM2 mainly increased cycling and IFN-R fates when deficient or suboptimal. Analysis of expression dynamics of individual genes showed that hT2AB co-stimulated expression changes as soon as cell fates were determined, but did not alter the transcriptional identity of the terminal cell types.

We finally tested the effect of mT2AB on brain biochemical analytes and histological images in the 5XFAD mice after repeated injections every 3 days for a short period of time. mT2AB treatment resulted in the greatest increases in CCL4, CXCL10 and IL-1β, which are produced by microglia, in TREM2^{R47H}-5XFAD mice. This is consistent with the scRNA-seq data suggesting that mT2AB induces greater activation of microglia in TREM2^{R47H}-5XFAD than in TREM2^{CV}-5XFAD mice. Female 5XFAD mice carrying TREM2^{R47H} and TREM2^{CV} showed greater levels of amyloid deposition relative to their male counterparts, which is consistent with the observations of greater hT2AB induced shifts in males versus females. We conclude that the amplitude of hT2AB-mediated effects on microglia are shaped by their basal cycling and differentiation status, pre-existing TREM2 engagement and basal microglia activation prior to mAb-mediated stimulation.

### 7.1.4. Results

hT2AB is an Agonistic mAb Specific for Human TREM2

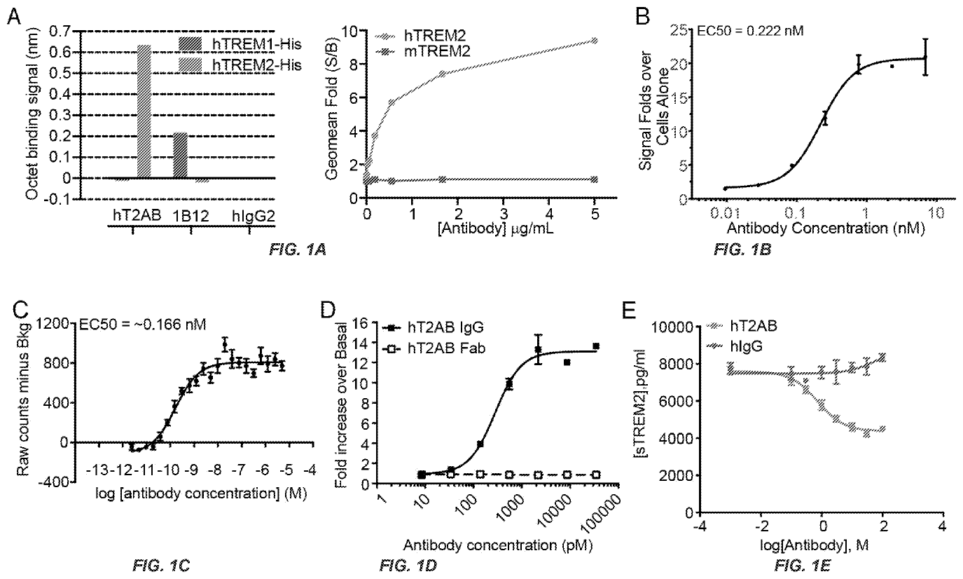

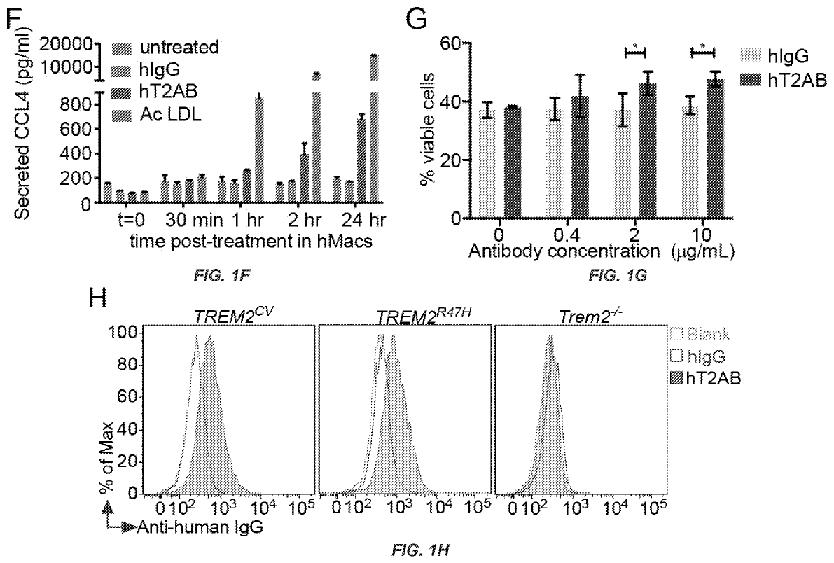

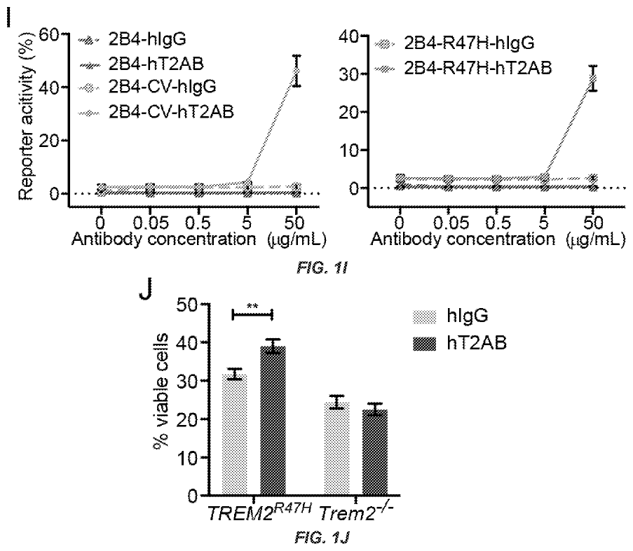

hT2AB was generated by gene gun-mediated immunization of XenoMouse® animals (29) with cDNA encoding human TREM2 and DAP12. Monoclonal antibody hT2AB was selected from a panel of agonistic anti-TREM2 antibodies. hT2AB selectively bound purified human TREM2 (affinity (K_{D})=50 nM) (FIG. 1A). Functional potency was determined by pSyk induction in HEK293 cells expressing human TREM2 and DAP12 (clone G13) and in human monocyte-derived macrophages (hMacs), revealing an EC50 of 222 pM and 166 pM, respectively (FIGS. 1-1C). hT2AB induction of intracellular signaling depended on bivalent binding and cross-linking of TREM2, as demonstrated by the lack of activity of its monomeric antigen-binding fragment (Fab) (FIG. 1D). Furthermore, release of sTREM2 following stimulation of hMacs was reduced by hT2AB (FIG. 1E).

To examine the effect of hT2AB-mediated activation of TREM2 on primary macrophages, hMacs were stimulated with hT2AB, isotype control, or acetylated LDL as a positive control, followed by assessment of chemokines released in culture supernatants at various timepoints after stimulation. hT2AB induced a time-dependent increase in the amount of CCL4 released by human macrophages (FIG. 1F), demonstrating that hT2AB activates primary macrophages. We previously showed that TREM2 enables macrophage survival in cultures with limiting concentrations of CSF1 (30) and tool mouse TREM2 antibodies that induce pSyk, also boost in vitro survival of macrophages under the same challenge conditions (23). Thus, we also tested whether hT2AB impacts in vitro survival of macrophages after CSF1 withdrawal. We prepared bone marrow macrophages (BMMs) from TREM2^{CV} mice by culture with CSF1, and then tested their survival in the presence or absence of hT2AB, 48 hours after removal of CSF1 from the medium (FIG. 1G). Indeed, hT2AB sustained survival of BMM in a dose-dependent manner.

Finally, we sought to determine whether the AD-associated R47H mutation affects hT2AB binding and/or signaling. First, we demonstrated that hT2AB stained BMM prepared from both TREM2^{CV} and TREM2^{R47H} mice (FIG. 1H). Then, we tested whether hT2AB can induce the expression of GFP in the Ca²⁺-NFAT-driven reporter cell line 2B4 stably transfected with either TREM2^{CV} or TREM2^{R47H} together with DAP12 (FIG. 1I) (18). hT2AB induced GFP in both TREM2^{CV} and TREM2^{R47H} transfected reporter cells. Moreover, hT2AB sustained survival of BMM derived from TREM2^{R47H} mice cultured without CSF1 (FIG. 1J), conclusively demonstrating that hT2AB activates both TREM2^{CV} and TREM2^{R47H}

hT2AB can Cross the BBB and Attain Effective Brain Concentrations

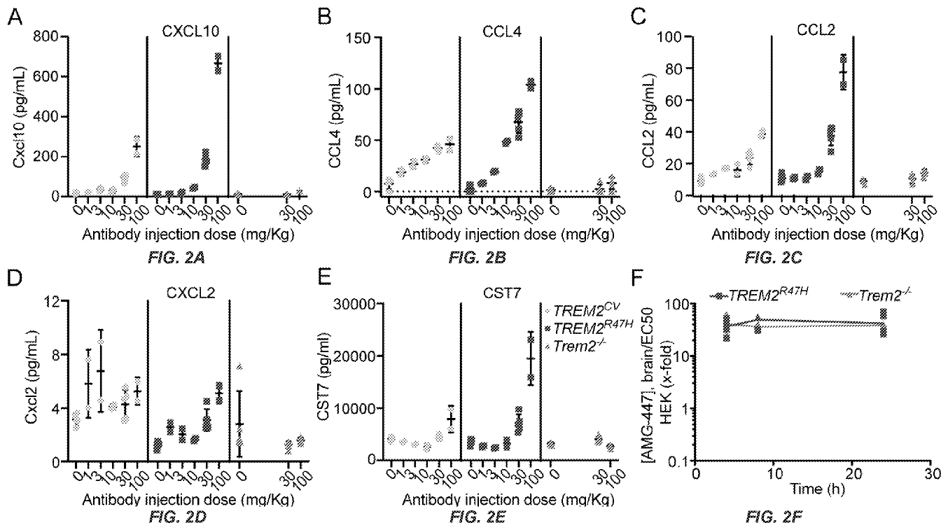

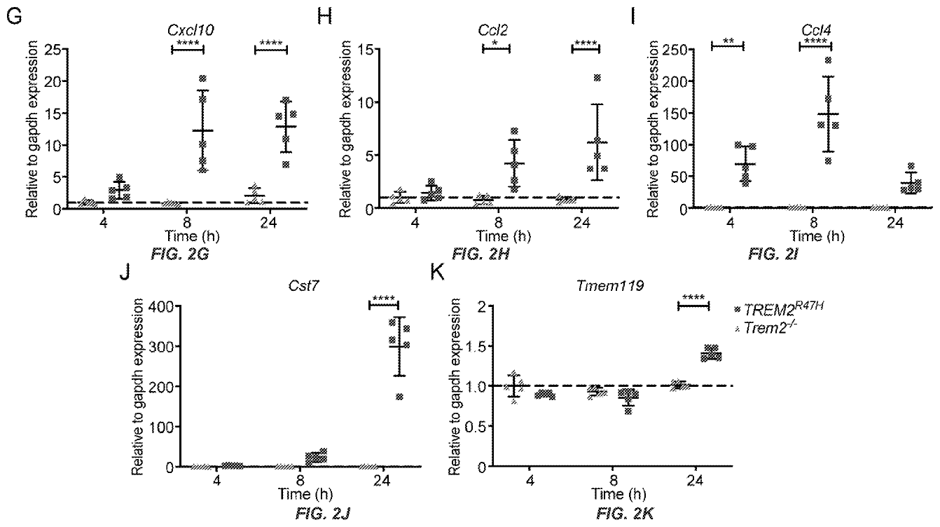

Since peripherally administered mAbs cross the blood-brain barrier (BBB) poorly, we performed pharmacokinetic/pharmacodynamic studies to define a dosing scheme that ensures effective concentrations of hT2AB in the brain. Groups of TREM2^{CV}, TREM2^{R47H} and Trem2^{-/-} mice were injected intravenously (i.v.) with different doses of hT2AB. After 24 hours, we measured the concentrations of CXCL10, CCL4, CCL2, CXCL2 and CST7 by MSD in brain lysates as proxies of microglia activation. hT2AB amplified all parameters at a dose between 30-100 mg/kg (FIGS. 2A-2E). TREM2^{R47H} mice were slightly more responsive than TREM2^{CV} mice. No response above baseline was noted in Trem2^{-/-} mice. A time course of hT2AB concentration in the brain lysates after single i.v. injection of 30 mg/kg in TREM2^{R47H} and Trem2^{-/-} mice revealed that the hT2AB brain concentration was 25-fold higher than its EC50 in clone G13 cells at all time points (4, 8 and 24 hrs) (FIG. 2F). Accordingly, an increase of CXCL10, CCL2, CCL4, CST7, and TMEM119 mRNAs was detectable in lysates of TREM2^{R47H} but not Trem2^{-/-} brains 8 hours after injection and further increased after 24 hours (FIGS. 2G-2K). We conclude that a single injection of 30 mg/Kg of hT2AB is sufficient to activate microglia in vivo for at least 24 hrs.

scRNA-Seq Reveals Four Microglia Trajectories in Control hIgG1-Treated TREM2^{CV}-5XFAD Mice

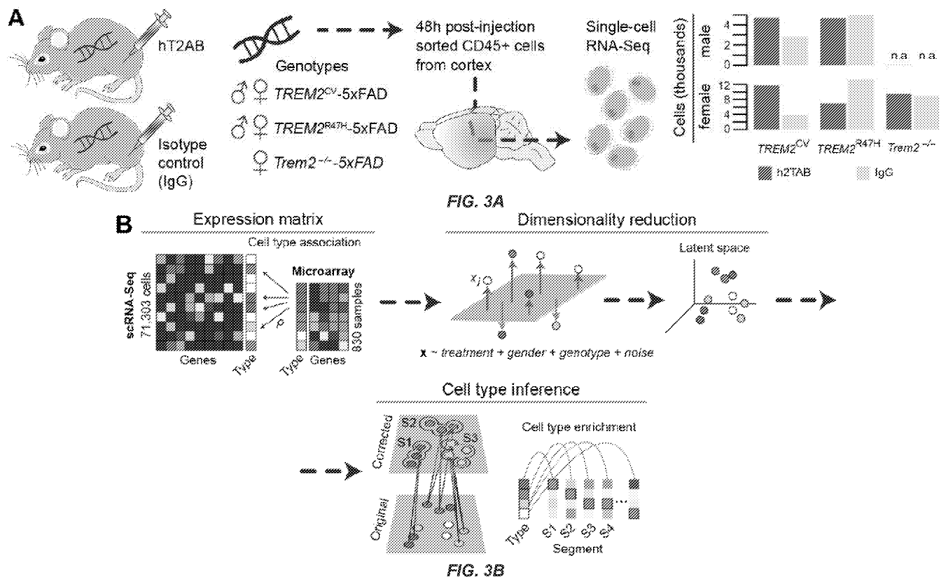

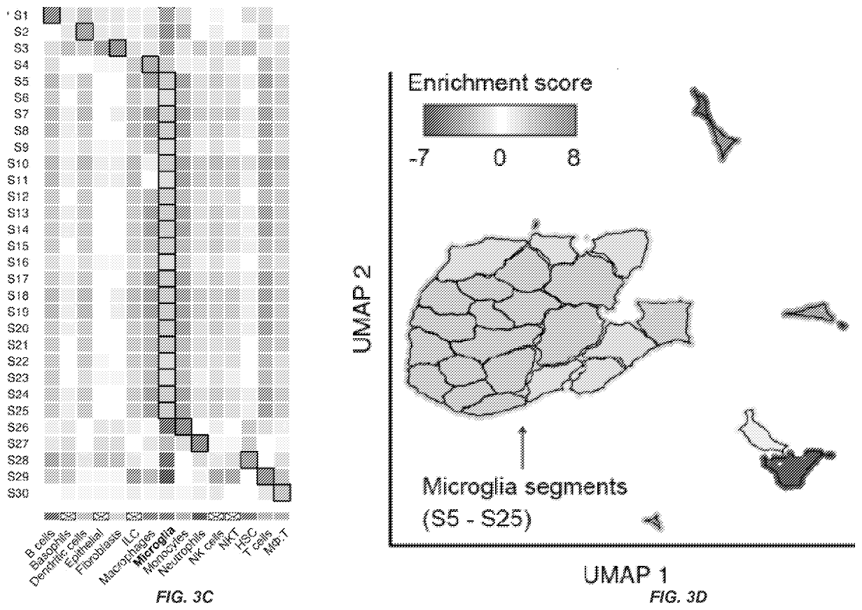

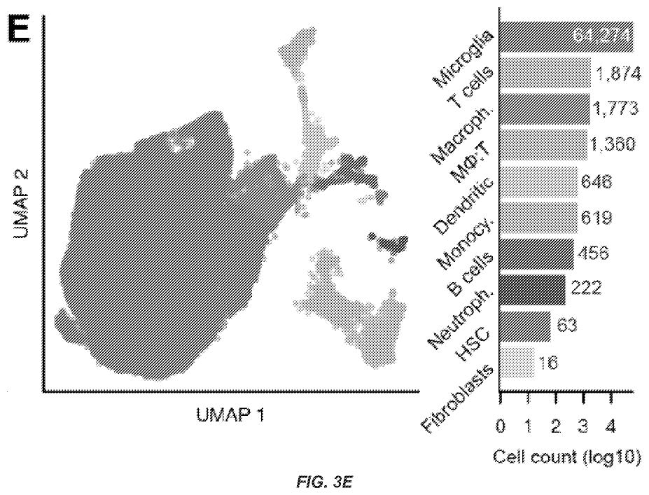

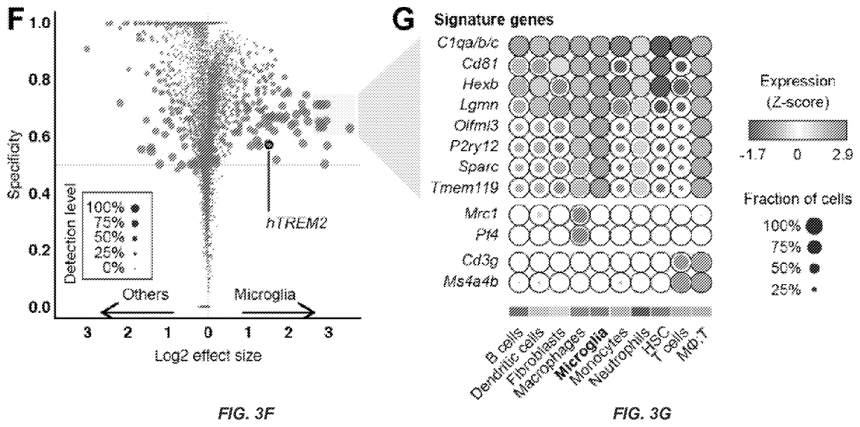

To examine the impact of hT2AB on microglia in vivo we resorted to scRNA-seq. We injected a single dose of hT2AB or control hIgG1 into the peritoneal cavity of 8-month-old 5XFAD mice crossed to either TREM2^{CV} (TREM2^{CV}-5XFAD) or TREM2^{R47H} (TREM2^{R47H}-5XFAD) mice. Both females and males were included and we also injected female Trem2^{-/-} mice to control for off target effects of hT2AB (FIG. 3A). Mice were sacrificed 48 hours after injection. Assessment of antibody levels in the cerebellum confirmed that hT2AB crossed the BBB and reached the brain parenchyma (TABLE S1). CD45⁺ cells were isolated from brain cortices and submitted for scRNA-seq using the 10× Genomics Chromium platform (FIG. 3A). 71,303 cells passed a rigorous multi-step quality control process (FIG. S1). We applied a supervised approach to initially classify cells into major immune cell subtypes by matching single-cell expression profiles to ImmGen gene signatures (31) (FIG. 3B). A lower-dimensional latent space correcting for treatment, sex, and genotype covariates, as well as blocking technical confounders was derived from the cellular expression profiles. A graph encoding cellular relationships was generated using the Jaccard similarity coefficient on each cell's nearest neighborhood and unbiasedly segmented using Louvain's community detection method. Each cell was assigned to the most enriched cell type in its segment resulting in a total classification of 10 major immune cell populations (FIGS. 3C-3E). Microglia accounted for more than 90% of total cells. The remaining cells were composed of T cells, macrophages, dendritic cells, monocytes, B cells, neutrophils, cycling cells, fibroblasts, as well as a population of cells that had a mixed expression profile of macrophages and T cells (MΦ:T), perhaps capturing the interaction of T cells infiltrating the brain of mice accumulating Aβ (32). Microglia differentially expressed common marker genes, including P2ry12, Hexb, Tmem119, and C1q family genes, amongst others, that were absent in other cell populations (FIGS. 3F-G). These genes were also preferentially expressed in a small cluster identified as perivascular macrophages that had increased Mrc1 and Pf4 expression. Notably, monocytes and perivascular macrophages were not found significantly impacted due to hT2AB treatment. The relative fraction of sampled monocytes or perivascular macrophages from mice treated with either control hIgG1 or hT2AB remained unchanged (hIgG1/hT2AB ratio for monocytes:TREM2^{CV}-5XFAD=1.3%/0.9%, TREM2^{R47H}-5XFAD=1.0%/0.9%, Trem2^{-/-}-5XFAD=0.7%/0.4%; macrophages: TREM2^{CV}-5XFAD=3.0%/3.0%, TREM2^{R47H}-5XFAD=2.4%/2.4%, Trem2^{-/-}-5XFAD=2.3%/1.8%), and no genes were found differentially expressed between treatment cohorts (absolute effect size threshold >1.5).

TABLE S1 shows cerebellar concentration of hT2AB after a single i.p. injection. Analysis of hT2AB dosed samples in serum/brain showed exposure to hT2AB and the ratio of BBB passing for hT2AB was around 0.19%-0.76%. Genotypes: A=TREM2 CV-5XFAD; B TREM2 R47H-5XFAD; C=Trem2−/−-5XFAD

| | | | | | % Ratio |
|---|---|---|---|---|---|
| | | Treatment | hT2AB (nM) | | (Concentration |
| Genotype | Gender | (48 h) | Serum | Brain | Br/Sr) |
| A | M | hT2AB | 2153.33 | 6.56 | 0.30 |
| A | M | hT2AB | 2020.00 | 3.75 | 0.19 |
| A | F | hT2AB | 394.67 | 2.98 | 0.76 |
| A | F | hT2AB | 2450.00 | 7.27 | 0.30 |
| A | F | hT2AB | 2793.33 | 7.53 | 0.27 |
| B | M | hT2AB | 1773.33 | 8.00 | 0.45 |
| B | F | hT2AB | 1733.33 | 4.18 | 0.24 |
| B | F | hT2AB | 1673.33 | 9.07 | 0.54 |
| C | F | hT2AB | 2100.00 | 7.20 | 0.34 |
| C | F | hT2AB | 1993.33 | 5.42 | 0.27 |
| C | F | hT2AB | 2280.00 | 5.69 | 0.25 |

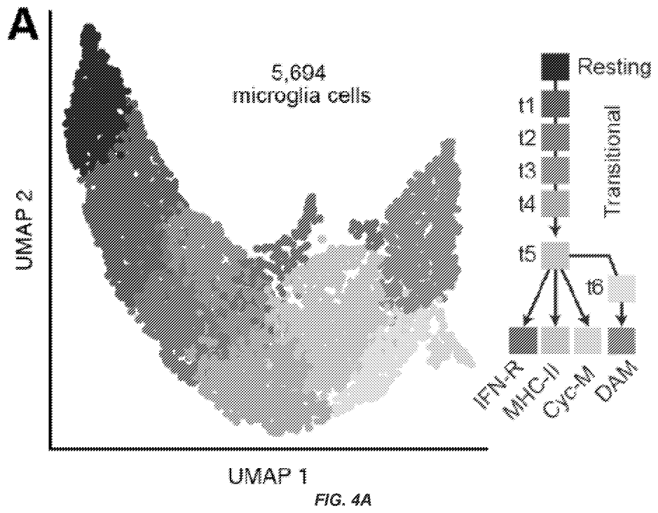

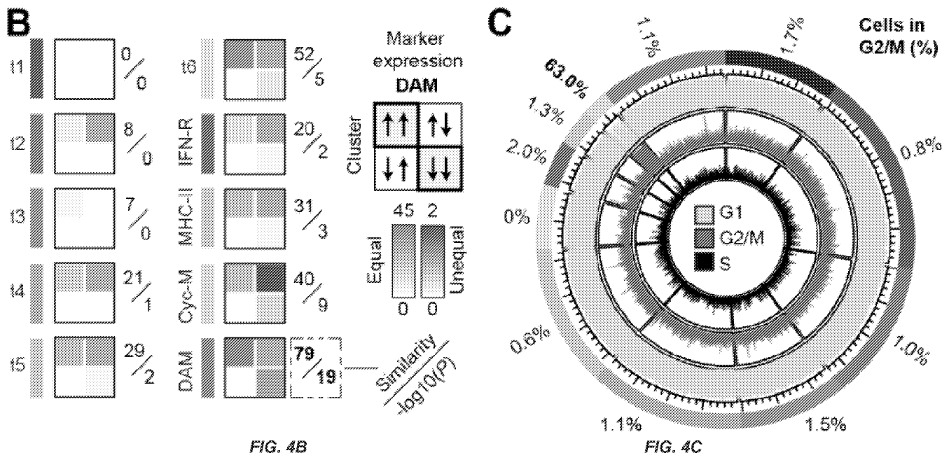

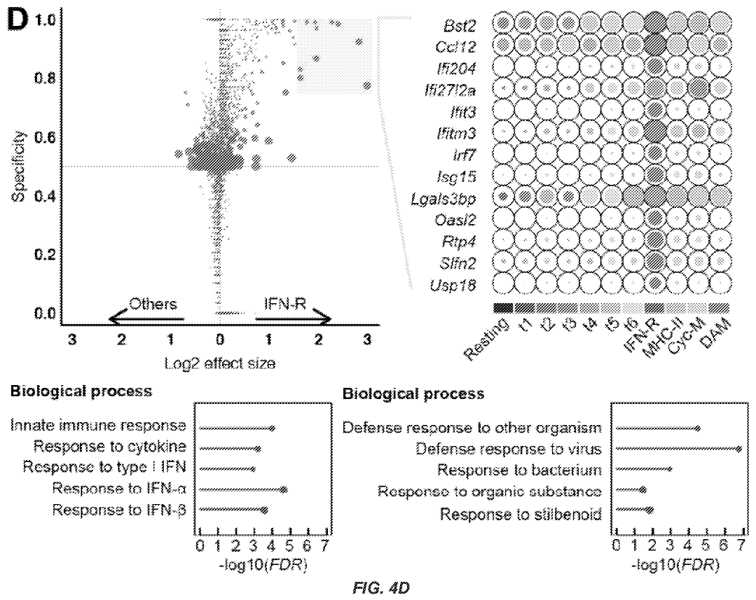

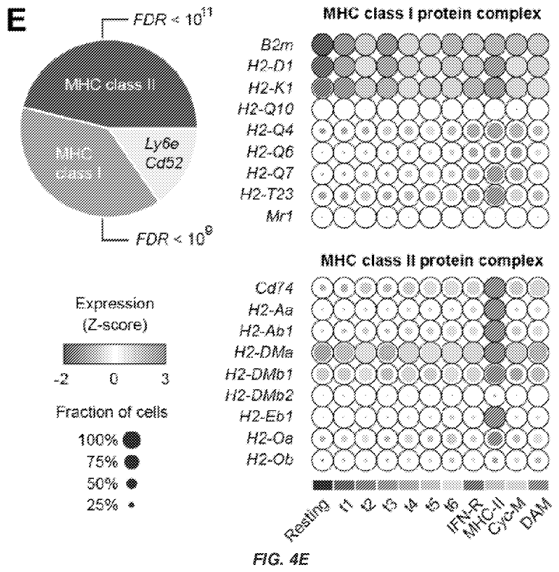

We next defined the baseline heterogeneity for 5,694 microglia cells from control hIgG1-injected TREM2^{CV}-5XFAD mice. We used diffusion maps to learn a lower-dimensional manifold best representing the latent temporal axis in the data. Clustering using Louvain's community detection method revealed 11 cell states, which were subsequently unbiasedly placed along a maximum parsimony tree resembling branching cell activation continua. The resulting trajectory originated from homeostatic microglia (highly expressing Tmem119, P2ry12, and Cx3cr1), progressed through 5 intermediate stages of differentiation (t1-t5) and then branched into 4 distinct terminal types: interferon-responsive microglia (IFN-R), MHC-II-expressing microglia (MHC-II), cycling microglia (Cyc-M) and a t6 stage that further differentiated into a DAM cluster (FIG. 4A). By scoring transcriptome similarities and inequalities with the DAM reported by Keren-Shaul et al., (9), we found an increasing expression resemblance along the trajectory from t1 to t6 and DAM clusters reported here, culminating in a significant similarity between terminal DAMs from both studies (P-value=1.4×10⁻¹⁹; FIG. 4B). We predicted the cell cycle state of each cell using a machine learning method that was trained on pairs of cell cycle state marker expression (33). Cyc-M evinced a significant enrichment of cells (63.0%) in G2/M phase across all clusters (average percentage in remaining clusters=1.1%; FIG. 4C). IFN-R microglia most abundantly expressed interferon-stimulated genes (ISG), such as Bst2, Ifit3, Ifitm3 and Isg15 (FIG. 4D). GO terms enrichment analysis corroborated the expression of a gene program induced by IFNs, particularly type I IFNs, i.e. IFNα and IFNβ (FIG. S2). Among all clusters, MHC-II microglia expressed the highest levels of MHC class II pathway genes, together with classical and non-classical MHC class I genes (FIG. 4E). Interestingly, this subset also expressed high levels of GPI-linked Cd52 and Ly6e, both of which have been associated with immunoregulation (34). Notably, IFN-imprinted and MHC-II presenting clusters were previously reported in another mouse AD model (35). Overall, our unbiased baseline scRNA-seq analysis of microglia in control hIgG1-treated TREM2^{CV}-5XFAD mice revealed four distinct fates in the microglial response to Aβ plaques, which may reflect the engagement of different signaling pathways.

### TREM2 Variants Profoundly Impact Microglia Trajectories

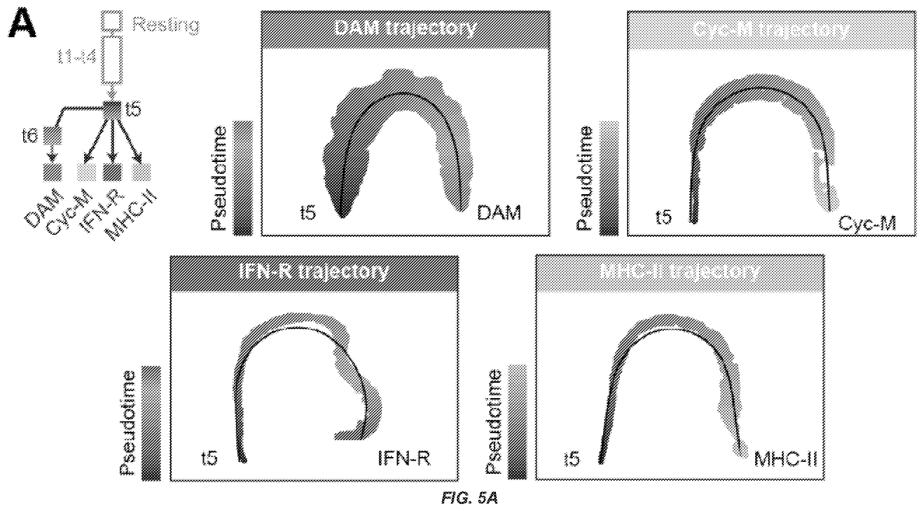

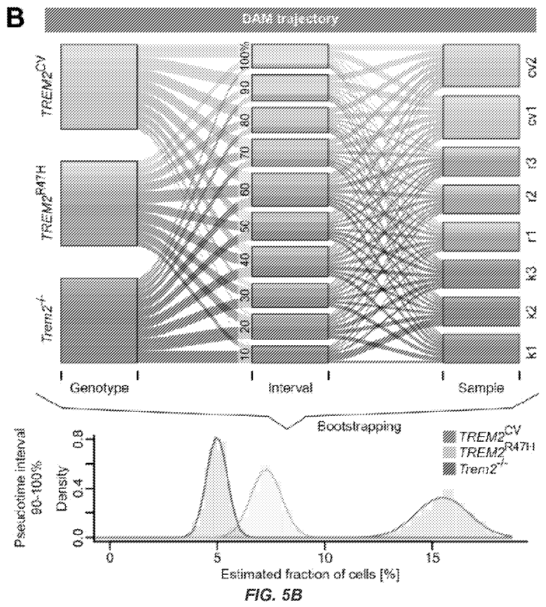

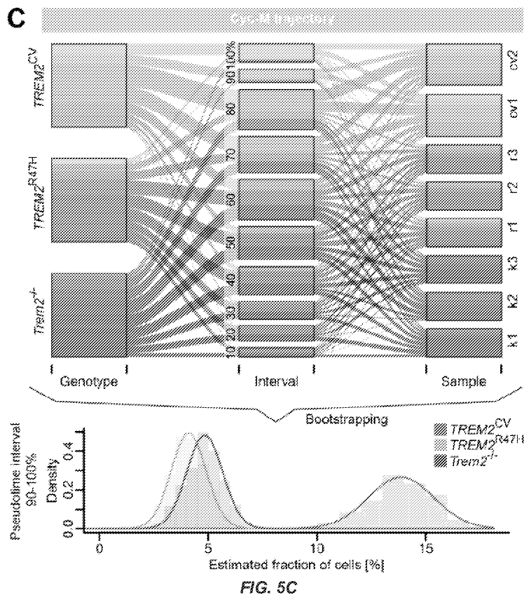

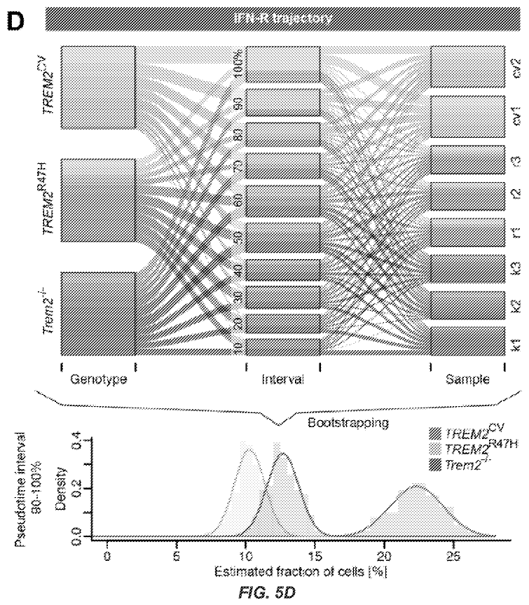

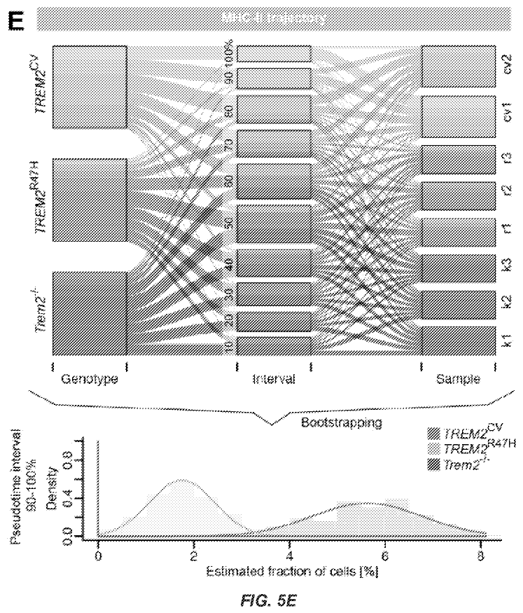

We intended to perform an integrative analysis on the impact of sex, genotype, and treatment on the differentiation of DAM, Cyc-M, IFN-R, and MHC-II microglia. For this purpose, we employed a machine learning-based approach to harmonize samples (FIG. S3) and we inferred trajectories from the branching cluster, t5, to each terminal end by fitting principal curves on the learned non-linear manifolds of all cells from all conditions (FIG. 5A). Using this method, we first assessed the baseline impact of TREM2^{CV}-5XFAD and TREM2^{R47H}-5XFAD genotypes on microglial trajectories. We particularly focused on females because they have more pronounced Aβ deposition than males (27, 28). Further, we included Trem2^{-/-}-5XFAD mice for further comparison. We split each cell type trajectory into 10 equally sized pseudotime intervals and quantified the number of cells from control hIgG1-treated female mice per interval. Proportions of cell populations were estimated using a Bootstrapping method to account for technical variance between replicates (FIGS. 5B-E). We found that for all four terminal time intervals, TREM2^{CV}-5XFAD mice had the highest fraction of cells (estimated mean±SE of 95% CI DAM: 15.5+0.1%, Cyc-M: 13.9+0.1%, IFN-R: 22.3±0.2%, MHC-II: 5.6±0.1%). This corroborated that TREM2 is required for full differentiation of DAM, consistent with previous studies (9), and extended this concept to the Cyc-M, IFN-R, and MHC-II fates induced by Aβ accumulation. TREM2^{R47H}-5XFAD mice harbored more late-stage DAMs and MHC-II cells (DAM: 7.3±0.1%, Cyc-M: 4.1±0.1%, IFN-R: 10.3±0.1%, MHC-II: 1.7±0.1%) than did Trem2^{-/-}-5XFAD mice (DAM: 5.0±0.0%, Cycling: 4.8±0.1%, IFN-R: 12.7±0.1%, MHC-II: 0.0±0.0%). This is in agreement with previous findings reporting that the TREM2^{R47H} mutant, although hypomorphic, is a partial loss-of-function with limited capacity to induce DAM maturation (19). Since TREM2^{R47H} poorly binds lipid ligands, constant TREM2 interaction with endogenous ligands is required to sustain the differentiation of DAM and to a minor extent MHC-II microglia.

Female Microglia are Prone to an IFN-R Fate in Control hIgG1-Treated TREM2^{CV}-5XFAD Mice

We compared the impact of sex on microglial trajectories in TREM2^{CV}-5XFAD mice. We did not observe a difference in proportions of late-stage DAMs (estimated mean±SE of 95% CI female: 15.5±0.1%, male: 14.5±0.1%; FIG. S4A). However, the representation of Cyc-M, IFN-R, and to a minor extend MHC-II cell types was biased. The relative fraction of cells in the terminal Cyc-M (FIG. S4B) and IFN-R (FIG. S4C) interval was considerably higher in females than in males (Cyc-M female: 13.9±0.1%, male: 8.1±0.1%; IFN-R female: 22.3+0.2, male: 17.0±0.2). Estimated terminal MHC-II cell fractions indicated a moderate tendency of this cell type to be more abundant in males than in females (female: 5.6+0.1%, male: 8.0±0.2%; FIG. S4D).

We also assessed the gene expression dynamics of the top 10 signature genes for each terminal type before and after the branching cluster t5 (FIGS. S4A-S4D). Gene expression was modeled as a function of pseudotime using negative binomial generalized additive models (36). This allows evaluation of gene expression patterns along the differentiation trajectory on a single-cell level and therefore, independent of cell sampling densities. For each cell type, signature genes showed a similar consecutive upregulation pattern towards the terminal end of each trajectory in both sexes. This implies that microglia types exhibit similar transcriptome compositions and thus, potentially perform qualitatively identical functions in males and females. However, microglia may be primed differently prompting a more dynamic microglial response, which ultimately results in a quantitative shift in the microglia population landscape between sexes. For example, the basal expression level of the interferon-stimulated gene Ifi27l2a was elevated in female mice along the Cyc-M and the IFN-R trajectory (Wald test on early and late terminal differences: Cyc-M P-value early=9.9×10⁻⁹, late=3.8×10⁻¹; IFN-R P-value early=1.0×10⁻⁴, late=1.8×10⁻²; FIG. S4B). Ifi27l2a is a regulator of the transcriptional activity of NR4A nuclear receptors, which coordinate cellular and systemic metabolic processes (37), as well as myeloid cell differentiation and their response to inflammatory stimuli (38-40). An induced basal expression level of Ifi27l2a may indicate increased cellular exposure to pathophysiological environmental cues. In fact, previous studies have shown that female 5XFAD mice accumulate more Aβ than male 5XFAD mice (27, 28). Accordingly, we also detected more insoluble Aβ in the brain of female than male TREM2^{CV}-5XFAD mice (FIG. S4E). Thus, altogether these data suggest that the enrichment of IFN-R and Cyc-M types in female TREM2^{CV}-5XFAD mice reflects a microglial response to more copious Aβ aggregates.

hT2AB Effects on Cycling, IFN-R and MHC-II Fates Depend on Pre-Existent Microglia State

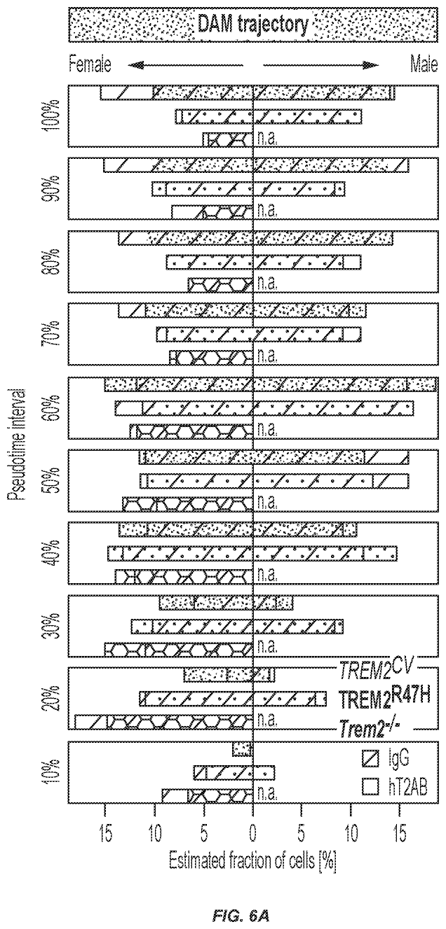

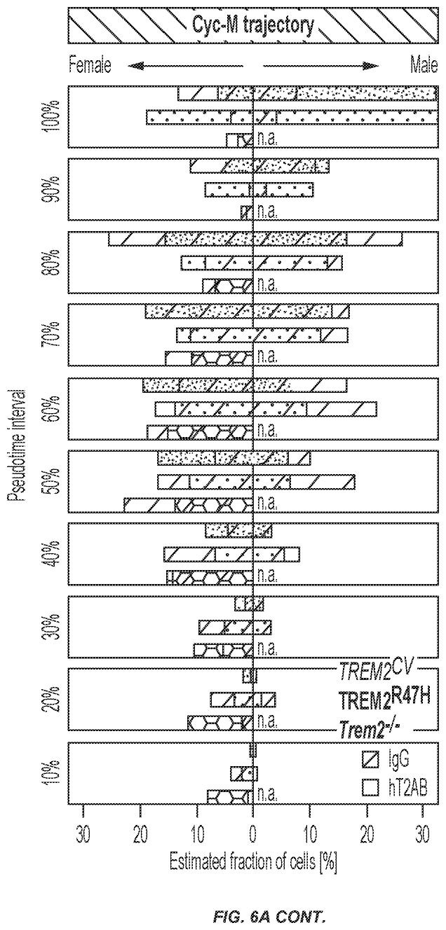

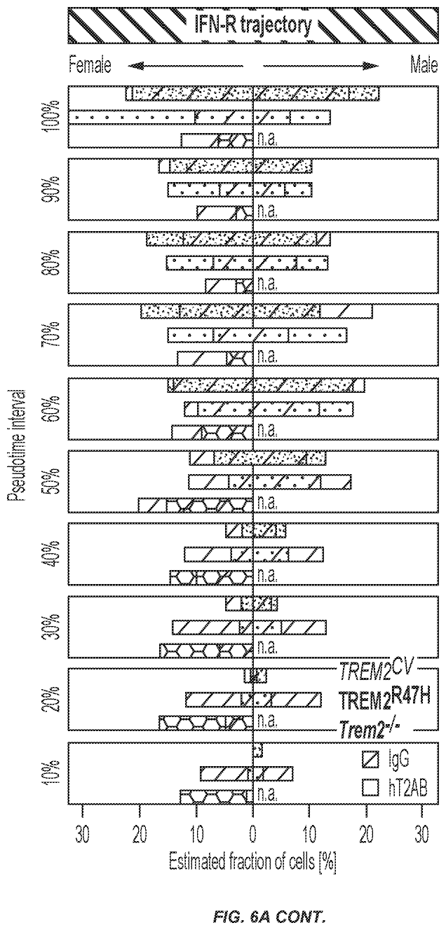

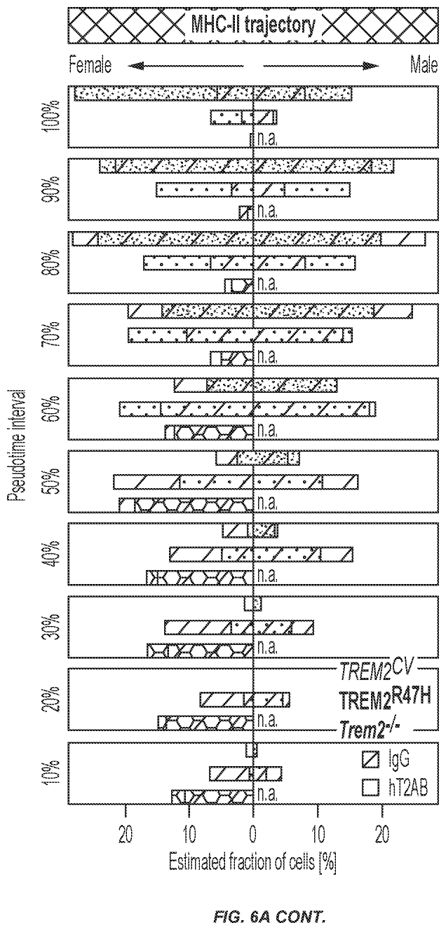

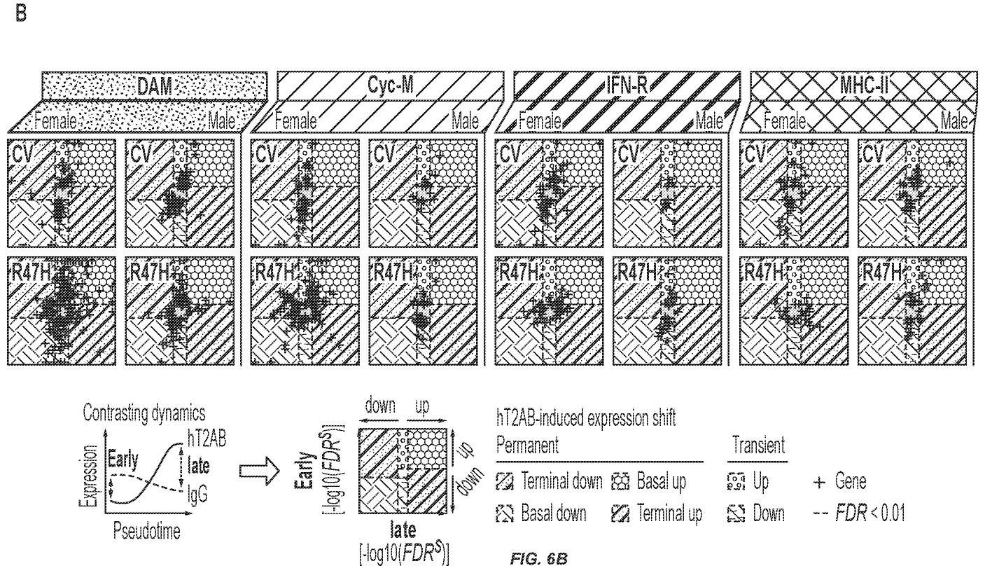

We next examined how hT2AB impacts microglial fates compared to control hIgG1 in TREM2^{CV}-5XFAD and TREM2^{R47H}-5XFAD mice of both sexes. To account for offsite treatment effects, we included data from treatment-matched Trem2^{-/-}-5XFAD mice. hT2AB did not induce expansion of DAM (estimated ratio of control hIgG1 to hT2AB cell fractions in the 90-100% pseudotime interval, R, of TREM2^{CV}/TREM2R^{47H}/Trem2^{-/-}-5XFAD females: R=0.6/1.0/0.9, males: R=1.0/1.0/n.a.). Interestingly, hT2AB treatment of TREM2^{CV}-5XFAD mice activated cell cycle re-entry of microglia in males (R=4.2), but microglia did not undergo additional cell cycle induction in females (R=0.5; FIG. 6A). An increased magnitude of proliferative response to hT2AB was evident in microglia from TREM2^{R47H}-5XFAD, as this population expanded in both female and male mice (female: R=4.9, male: R=7.9). Likewise, hT2AB induced the terminal IFN-R population in TREM2^{CV}-5XFAD males (R=1.3), TREM2R⁴⁷-5XFAD males (R=2.1) and TREM2^{R47H}-5XFAD females (R=3.2), but did not promote this cell fate in TREM2^{CV}-5XFAD females (R=0.9). Given that control hIgG1-treated TREM2^{R47H}-5XFAD mice have fewer Cyc-M and IFN-R microglia than TREM2^{CV}-5XFAD mice, and a similar disproportion is evident between TREM2^{CV}-5XFAD males and females, our result suggests that hT2AB activates both microglial proliferation and IFN-R differentiation either when acting as a surrogate ligand for TREM2R⁴⁷H, or when Aβ accumulation still limited. hT2AB does not appear to further increase the percentage of activated microglia beyond the robust induction of these activation states in female 5XFAD mice carrying TREM2^{CV}, which is consistent with prior descriptions of highly elevated DAM signatures in 5XFAD animals carrying wild-type TREM2. MHC-II microglia, which were relatively scarce in TREM2^{CV}-5XFAD mice of both sexes (though more were found in males than in females) underwent hT2AB-induced expansion in both sexes, but the response was more pronounced in females (female: R=4.9, male: R=1.9). Similarly, hT2AB enlarged the late-stage MHC-II populations in TREM2^{R47H}-5XFAD mice of both sexes (estimated ratio of control hIgG1 to hT2AB cell fractions in the 80-100% pseudotime interval of females: 4.1, males: 2.1). Overall, hT2AB triggered microglial cell cycle re-entry and replenished the IFN-R and MHC-II cell pools in TREM2^{R47H}-5XFAD mice. In TREM2^{CV}-5XFAD mice, hT2AB-induced effects were driven by pre-existing cell type compositions towards restoration of less abundant populations.

Anti-TREM2 Co-Stimulates the Expression of Single Genes within Terminal Microglia Types

Since our data described a developmental continuum, microglia clusters represented a mixture of cells, each encompassing a discrete quantity captured at different developmental stages. Thus, by comparing the cell clusters, gene expression differences would be obscured. To avoid this problem, we fitted the gene expression dynamics of each cell as it differentiates from the branching point towards a terminal phenotype. The resulting expression model was independent of sampled cell fractions along the trajectory, allowing us to pinpoint underlying differential gene expression. Robustly detected genes were filtered per trajectory. We statistically compared the early start and the late end of each linage between hT2AB and control hIgG1 treated mice (FIG. 6B). Most hT2AB-induced gene expression changes occurred early in the pseudotime trajectory (mean=76.8%), while only a minority of these changes was manifest in the terminal cell type (mean=19.1%). This indicated that transcriptional shifts are mainly transient and that hT2AB acts as co-stimulator rather than as transcriptional modifier of terminal microglial phenotypes. We further found that, on average, 63.3% of transcriptional responses per trajectory were applicable to microglia carrying the TREM2R^{47H} hypomorph, which binds endogenous ligands less effectively but can be activated by hT2AB; this points to an activation state-dependent effect of hT2AB on microglial fate. Interestingly, the highest number of hT2AB-induced gene expression changes was detected along the DAM trajectory (average number of genes/total analyzed: DAM=552/1735, Cyc-M=321/3353, IFN-R=187/5005, MHC-II=148/4508), indicating that this trajectory was stimulated, but hT2AB treatment did not lead to full differentiation of the cells to a terminal phenotype in the time course of this experiment. Overall, transcriptional changes did not linearly translate into expansion of terminal cell types due to their dependency on the differentiation status prior to hT2AB-mediated stimulation.

Short-Term Treatment with mT2AB Impacts Microglial Proliferation and Activation Markers in TREM2^{R47H}-5XFAD

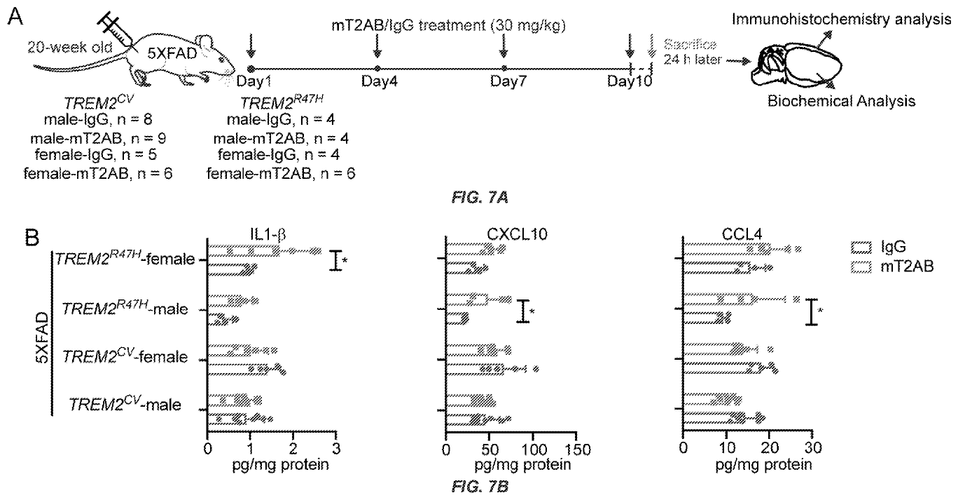

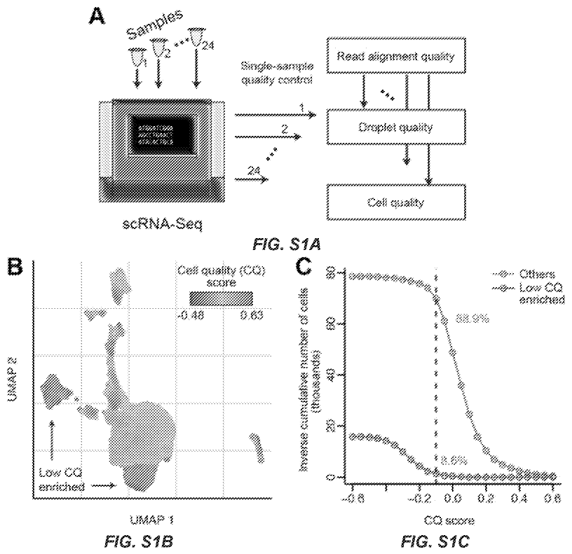

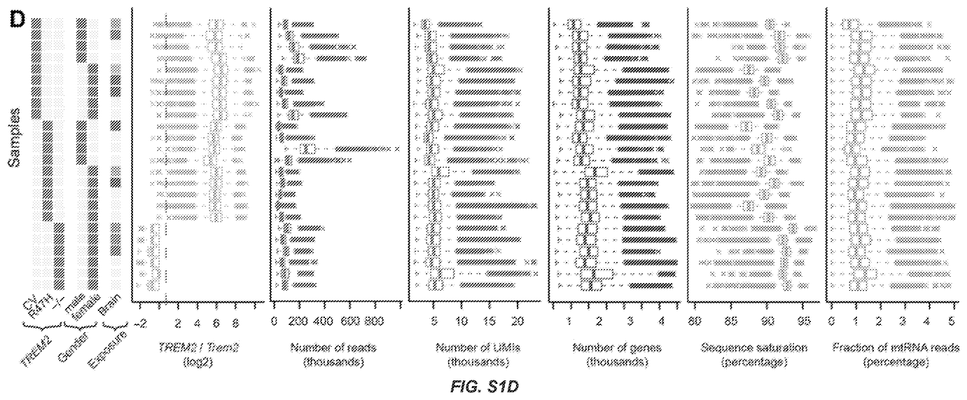

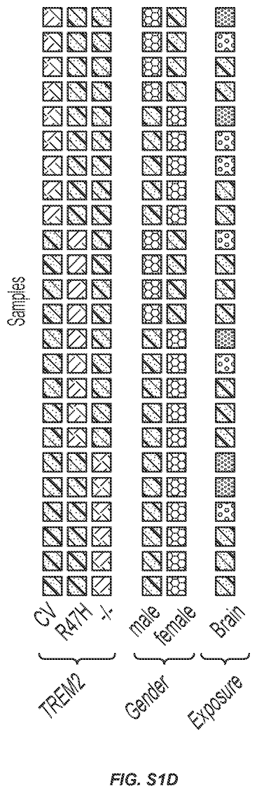

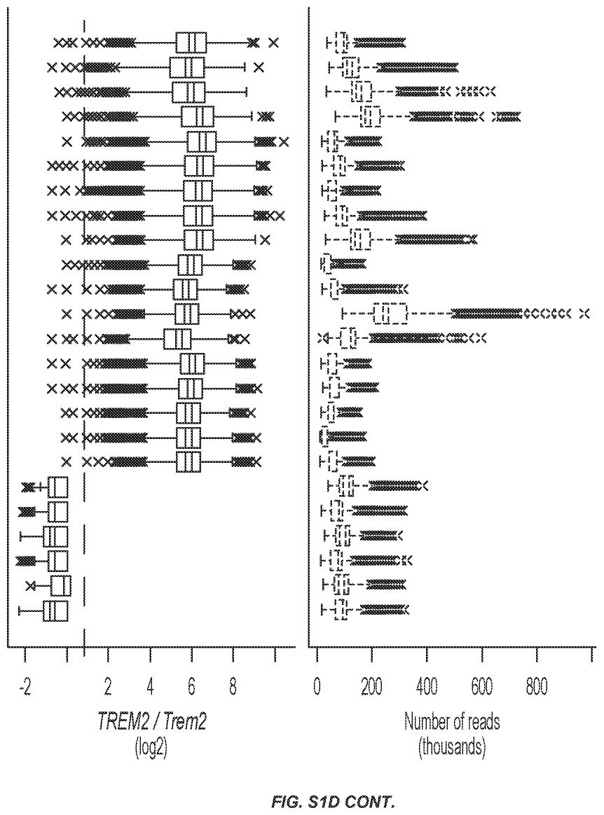

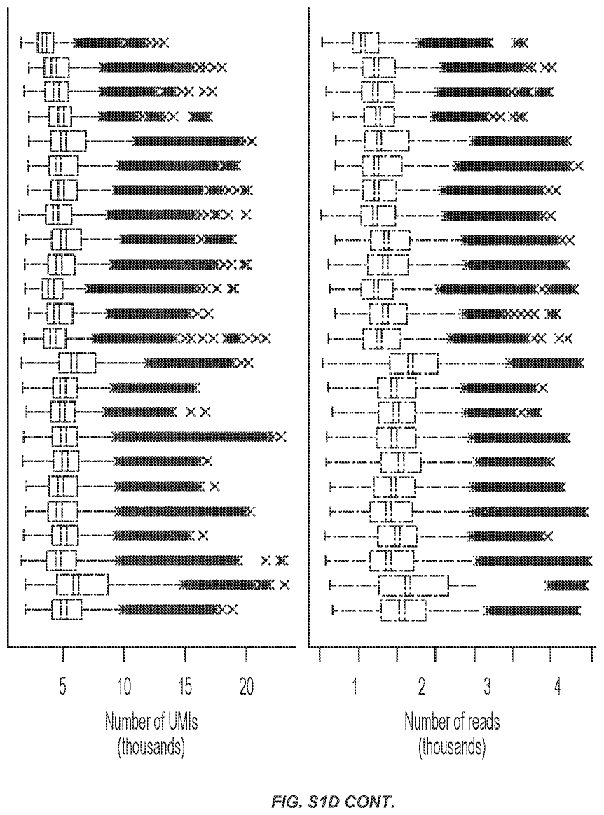

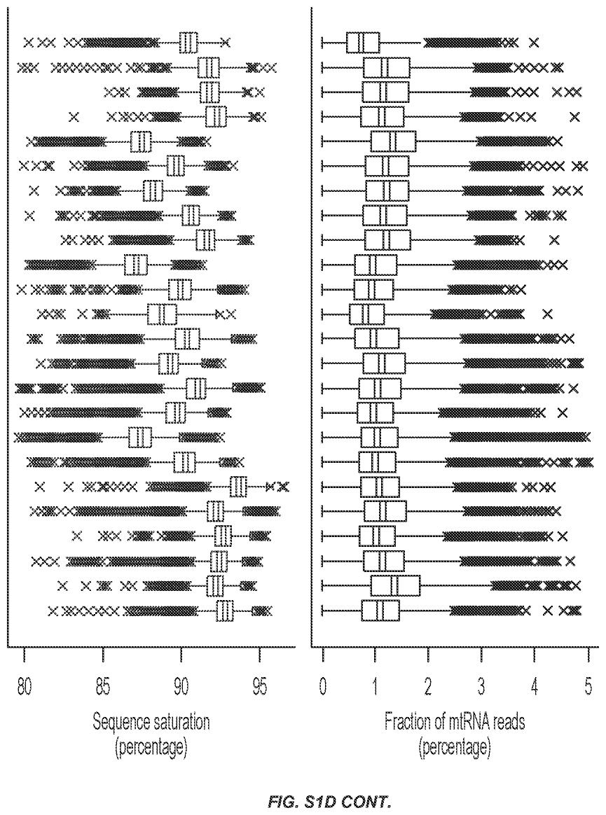

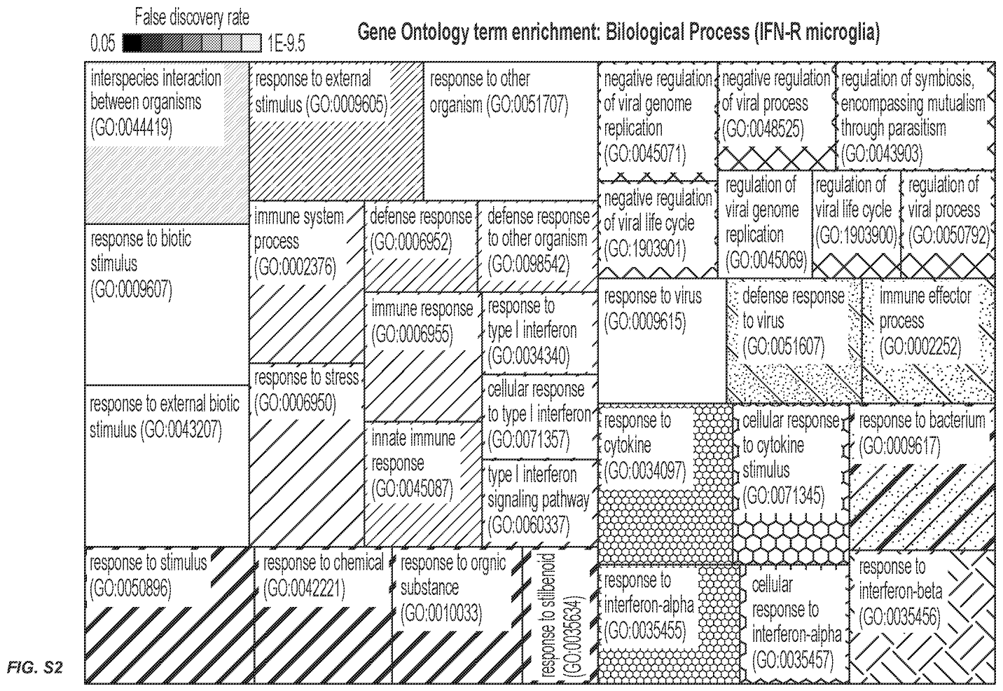

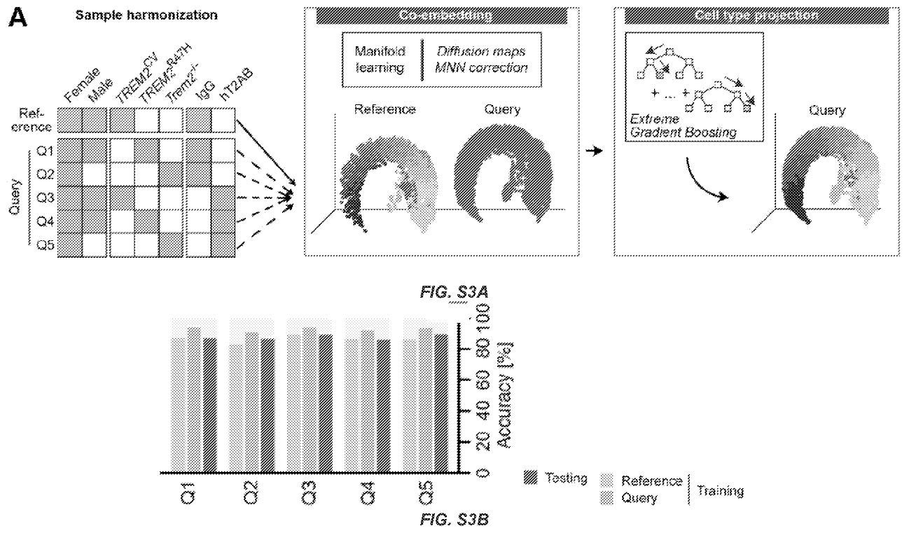

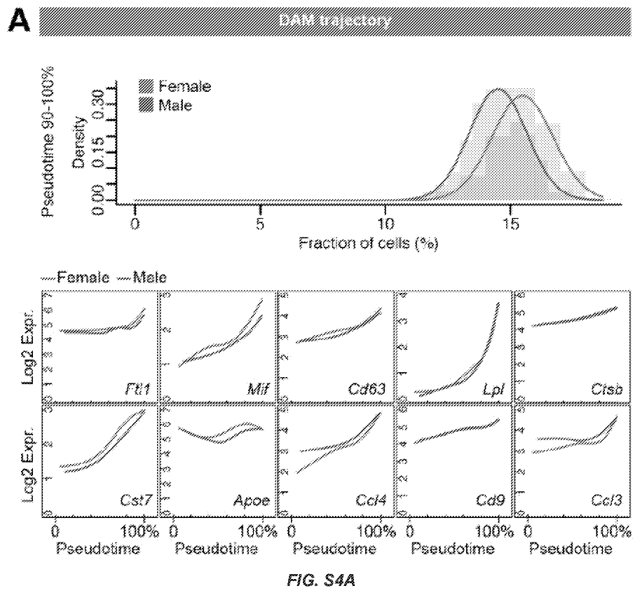

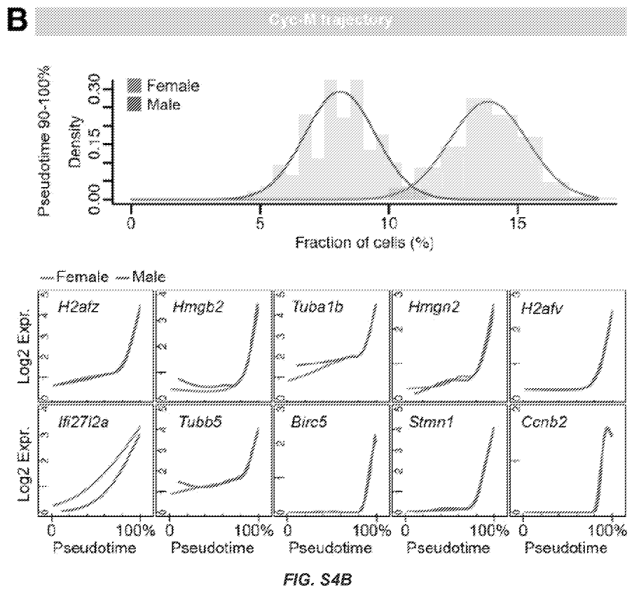

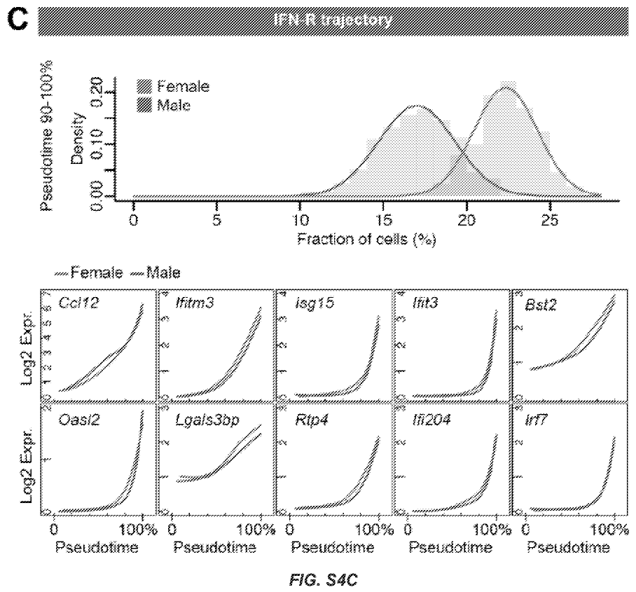

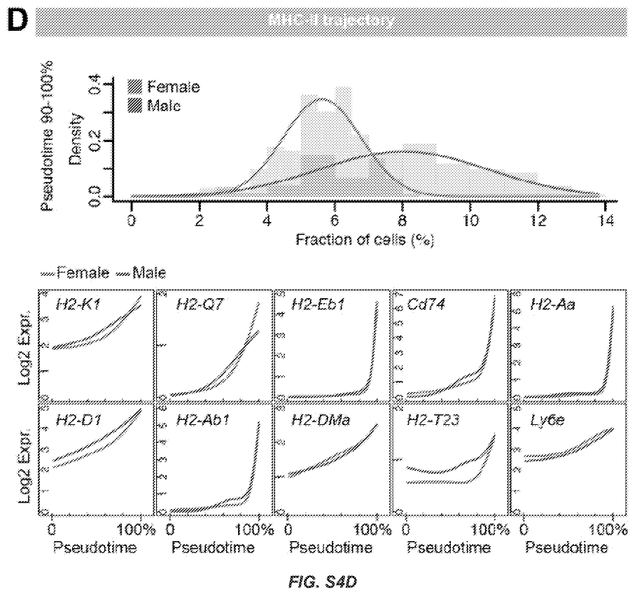

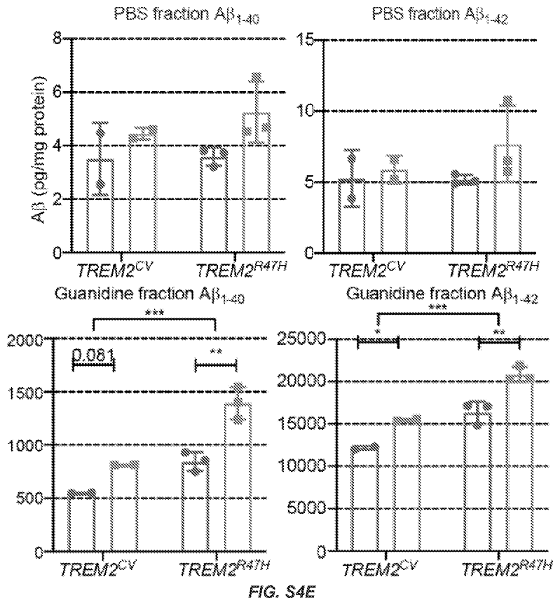

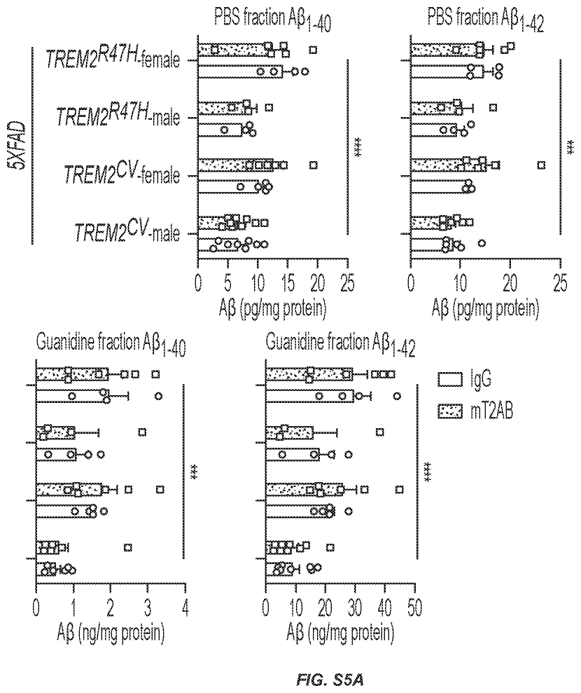

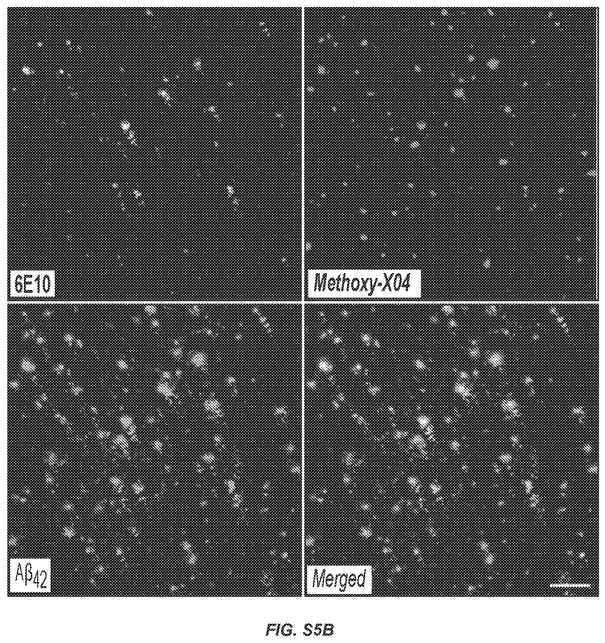

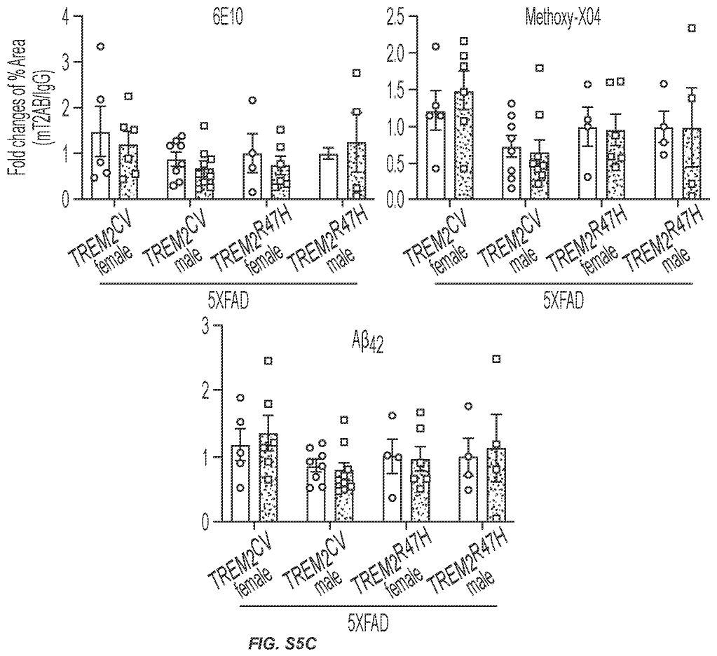

We next sought to determine the impact of anti-TREM2 treatment on brain biochemical and histological markers in 5XFAD animals. For this purpose, we utilized a murine IgG1 constant region chimeric variant of hT2AB (mT2AB). 5-month-old TREM2^{CV}-5XFAD and TREM2^{R47H}-5XFAD mice of both sexes were injected with a 30 mg/kg dose of mT2AB or control mIgG1 in the peritoneal cavity every 3 days for 10 days (FIG. 7A). At the end of the experiments, brains were divided into two halves: one half was lysed for biochemical measurement of soluble markers of microglial activation and proliferation including chemokines and cytokines, as well as Aβ peptides 1-40 and 1-42; the second half was sectioned to analyze Aβ coverage by confocal microscopy using anti-Aβ and Methoxy-04. Changes in chemokines and cytokines in hTREM2 transgenic mice on a 5XFAD background were consistent with those initially observed in hTREM2 transgenic mice on a wild-type background (FIG. 2), with induction of CCL4, CXCL10, and IL-10 observed in TREM2^{R47H}-5XFAD mice (FIG. 7B). Notably, mT2AB induced levels of CCL4, CXCL10 and IL-10 in TREM2^{R47H}-5XFAD mice comparable to the levels observed in TREM2^{CV}-5XFAD mice (FIG. 7B). Given that CCL4, CXCL10, and IL-1β are produced by microglia, these observations are consistent with scRNA-seq observations of increased proliferation and activation particularly of microglia expressing the TREM2^{R47H} variant. Aβ quantitation identified increased levels of amyloid deposition in females relative to males in both TREM12^{CV} and TREM2^{R47H} mice (FIG. S5A). This result corroborated prior observations (25, 26) and extended them to mice carrying human TREM2, providing a potential explanation for the greater relative increases of microglial activation response genes in males observed after hT2AB treatment. Short term treatment with mT2AB had no detectable impact on the total amount of plaque or soluble Aβ in either male or female mice, as measured by biochemical assessment of soluble or insoluble Aβ load (FIG. S5A) and staining with anti-Aβ and Methoxy-04 (FIGS. S5B-C). This is consistent with recent studies in which longer term treatment with TREM2 agonist antibodies resulted in robust induction of microglial barrier function and prevention of downstream neurotoxicity, without altering total amyloid quantitation (25). It is possible that differences in the microglial response to amyloid or other AD pathologies can shed light on historical observations that total amyloid burden correlates poorly with cognition and that high levels of amyloid deposition can be observed in cognitively normal older adults (41, 42).

### 7.1.5. Discussion

In this study we investigated the impact of amyloid deposition and an anti-human TREM2 agonist antibody on microglia activation, proliferation, and gene expression in TREM2^{CV}-5XFAD and TREM2^{R47H}-5XFAD mice. We first established a high-resolution single-cell profile of microglia in control hIgG1-treated TREM2^{CV}-5XFAD mice, demonstrating that microglia progressively differentiate from a homeostatic state into 4 distinct types in response to Aβ accumulation. These types include the previously reported DAM (9), IFN-R, MHC-II (35), as well as Cyc-M microglia. We showed that the relative distribution of microglial types depends on TREM2 genotype and mouse sex. In mice not treated with hT2AB, TREM2^{CV} was required for optimal differentiation of all types, which were conversely underrepresented in mice carrying the TREM2^{R47H} variant. Furthermore, IFN-R and Cyc-M microglia were more abundant in female than male TREM2^{CV}-5XFAD mice, most likely reflecting the exacerbated Aβ accumulation in females. We next established the impact of hT2AB on microglial fates, demonstrating two major effects: hT2AB promoted the expansion of Cyc-M microglia in mice with lower basal proliferation, including TREM2^{R47H}-5XFAD mice of both sexes and TREM2^{CV}-5XFAD male mice; moreover, hT2AB replenished the IFN-R and MHC-II cell pools in TREM2^{R47H}-5XFAD mice. Finally, in a short-term multi-dose scheme, mT2AB led to elevated brain content of chemokines in TREM2^{R47H}-5XFAD mice, which paralleled hT2AB-induced expansion of microglia.

One important conclusion of our study is that the activity of TREM2 seems saturable. Mice expressing TREM2^{R47H}, which is unable to effectively bind physiological ligands, had a clear defect in microglia cycling. However, engagement of TREM2^{R47H} with a surrogate ligand, such as hT2AB, markedly increased microglia proliferation. A similar result was recently corroborated with a different anti-human TREM2 mAb (25). In contrast, in mice expressing TREM2^{CV}, which binds endogenous ligands and promotes normal basal proliferation, hT2AB promoted only a modest increase of cycling of male microglia, which tend to proliferate less than female microglia because of less exposure to Aβ accumulation. This observation is important for future therapeutic applications of anti-TREM2 antibodies, as it suggests that these antibodies may be able to restore the impaired activation state observed in patients with hypofunctional mutations in TREM2 or that otherwise fail to mount a robust DAM response, as has been observed in human AD post-mortem brains. It also suggests that activation of microglia by TREM2 agonists is less likely to occur in the absence of injurious stimuli such as Aβ or alternate physiological TREM2 ligands.

It was previously shown that TREM2 is essential to induce full differentiation of DAM (9). Consistent with this, TREM2^{R47H}-5XFAD and Trem2^{-/-}-5XFAD mice had fewer DAM. Our data extend this concept to IFN-R and MHC-II microglia, which were also less abundant in TREM2^{R47H}-5XFAD and Trem2^{-/-}-5XFAD mice than in TREM2^{CV}-5XFAD mice. Importantly, our study also demonstrates that TREM2 signaling, although necessary, is not sufficient to induce the various terminal microglial types, which is consistent with prior reports of two-step activation of the DAM phenotype (9). Microglia cell fate is first driven by shifting neuropathological conditions that trigger signaling pathways that furcate and coopt TREM2 to sustain expansion towards four terminal cell types. Accordingly, hT2AB induced transcriptional changes proximal to the branching point, as soon as cell fate decisions are made, suggesting that hT2AB may stimulate common pathways in each trajectory that facilitate progression towards terminal microglial types. Given our previous demonstration that TREM2 sustains the mTOR pathway, TREM2 signaling may costimulate pre-activated pathways by providing building blocks and energy required for microglial responses to Aβ or other injuries (43).

In a short-term multi-dose scheme consisting of frequent administrations of high mAb doses within 10 days, mT2AB induced responses consistent with microglial proliferation and activation which differed by TREM2 genotype and gender, presumably in relation to the reduced levels of TREM2 engagement in TREM2^{R47H} and male mice. Our report is consistent with a recent study in which a long term treatment of TREM2^{CV}-5XFAD and TREM2^{R47H}-5XFAD mice with a different anti-human TREM2 mAb resulted in expansion of metabolically active and proliferating microglial populations to a greater extent in TREM2^{R47H}-5XFAD than TREM2^{CV}-5XFAD (25) and in a 12-week experimental paradigm led to changes consistent with the induction of a more effective microglial barrier response including changes in plaque compaction, alterations of microglial-plaque association, and reductions in neurite dystrophy consistent with reduced neurotoxicity of AD.

In conclusion, our in vivo evaluation of the anti-human TREM2 mAb hT2AB shows that systemic administration of this antibody in TREM2^{CV}-5XFAD and TREM2^{R47H}-5XFAD in vivo 1) replenished the Cyc-M, IFN-R, and MHC-II pools in TREM2^{R47H}-5XFAD mice; 2) brought the representation of male Cyc-M and IFN-R microglia closer to that of females in TREM2^{CV}-5XFAD mice, in which microglia transitioning had already been robustly activated; and 3) co-stimulated common pathways in each trajectory that facilitate progression towards terminal microglial types.

### 7.1.6. References

The specific references below, and discussed herein, are incorporated in their entirety.
1. J. M. Long, D. M. Holtzman, Alzheimer Disease: An Update on Pathobiology and Treatment Strategies. Cell, 1-28 (2019).
2. D. J. Selkoe, J. Hardy, The amyloid hypothesis of Alzheimer's disease at 25 years. EMBO Mol. Med. 8, 595-608 (2016).
3. M. T. Heneka, Microglia take centre stage in neurodegenerative disease. Nat. Rev. Immunol. 19, 79-80 (2019).
4. R. E. Tanzi, L. Bertram, Twenty years of the Alzheimer's disease amyloid hypothesis: A genetic perspective. Cell 120, 545-555 (2005).
5. C. M. Karch, A. M. Goate, Alzheimer's disease risk genes and mechanisms of disease pathogenesis. Biol. Psychiatry 77, 43-51 (2015).
6. S. Carmona, J. Hardy, R. Guerreiro, The genetic landscape of Alzheimer disease, 1st Ed. (Elsevier B.V., 2018).
7. I. E. Jansen, et al., Genome-wide meta-analysis identifies new loci and functional pathways influencing Alzheimer's disease risk. Nat. Genet. 51, 404-413 (2019).
8. P. Yuan, et al., TREM2 Haplodeficiency in Mice and Humans Impairs the Microglia Barrier Function Leading to Decreased Amyloid Compaction and Severe Axonal Dystrophy. Neuron 90, 724-739 (2016).
9. H. Keren-Shaul, et al., A Unique Microglia Type Associated with Restricting Development of Alzheimer's Disease. Cell 169, 1276-1290.e17 (2017).
10. S. Krasemann, et al., The TREM2-APOE Pathway Drives the Transcriptional Phenotype of Dysfunctional Microglia in Neurodegenerative Diseases. Immunity 47, 566-581.e9 (2017).
11. Y. Zhou, et al., Human and mouse single-nucleus transcriptomics reveal TREM2-dependent and TREM2-independent cellular responses in Alzheimer's disease. Nat. Med. 26, 131-142 (2020).
12. K. Srinivasan, et al., Alzheimer's Patient Microglia Exhibit Enhanced Aging and Unique Transcriptional Activation. Cell Rep. 31, 107843 (2020).
13. T. K. Ulland, M. Colonna, TREM2—a key player in microglial biology and Alzheimer disease. Nat. Rev. Neurol. 14, 667-675 (2018).
14. P. Wunderlich, et al., Sequential proteolytic processing of the triggering receptor expressed on myeloid cells-2 (TREM2) protein by ectodomain shedding and γ-secretase-dependent intramembranous cleavage. J. Biol. Chem. 288, 33027-33036 (2013).
15. P. Thornton, et al., TREM 2 shedding by cleavage at the H157-S158 bond is accelerated for the Alzheimer's disease-associated H157Y variant. EMBO Mol. Med. 9, 1366-1378 (2017).
16. R. Guerreiro, et al., TREM2 variants in Alzheimer's disease. N. Engl. J. Med. 368, 117-127 (2013).
17. T. Jonsson, et al., Variant of TREM2 associated with the risk of Alzheimer's disease. N. Engl. J. Med. 368, 107-116 (2013).
18. Y. Wang, et al., TREM2 lipid sensing sustains the microglial response in an Alzheimer's disease model. Cell 160, 1061-1071 (2015).
19. W. M. Song, et al., Humanized TREM2 mice reveal microglia-intrinsic and -extrinsic effects of R47H polymorphism. J. Exp. Med. 215, 745-760 (2018).
20. Y. Wang, et al., TREM2-mediated early microglial response limits diffusion and toxicity of amyloid plaques. J. Exp. Med. 213, 667-675 (2016).
21. T. R. Jay, et al., Disease progression-dependent effects of TREM2 deficiency in a mouse model of Alzheimer's disease. J. Neurosci. 37, 637-647 (2017).
22. C. Y. D. Lee, et al., Elevated TREM2 Gene Dosage Reprograms Microglia Responsivity and Ameliorates Pathological Phenotypes in Alzheimer's Disease Models. Neuron 97, 1032-1048.e5 (2018).
23. Q. Cheng, et al., TREM2-activating antibodies abrogate the negative pleiotropic effects of the Alzheimer's disease variant Trem2 R47H on murine myeloid cell function. J. Biol. Chem. 293, 12620-12633 (2018).
24. K. Schlepckow, et al., Enhancing protective microglial activities with a dual function TREM 2 antibody to the stalk region. EMBO Mol. Med. 12 (2020).
25. S. Wang, et al., Anti-human TREM2 induces microglia proliferation and reduces pathology in an Alzheimer's disease model. J. Exp. Med. 217 (2020).
26. H. Oakley, et al., Intraneuronal β-amyloid aggregates, neurodegeneration, and neuron loss in transgenic mice with five familial Alzheimer's disease mutations: Potential factors in amyloid plaque formation. J. Neurosci. 26, 10129-10140 (2006).
27. K. R. Sadleir, W. A. Eimer, S. L. Cole, R. Vassar, Aβ reduction in BACE1 heterozygous null 5XFAD mice is associated with transgenic APP level. Mol. Neurodegener. 10, 1-16 (2015).
28. S. Bhattacharya, C. Haertel, A. Maelicke, D. Montag, Galantamine slows down plaque formation and behavioral decline in the 5XFAD mouse model of Alzheimer's disease. PLoS One 9, 1-12 (2014).
29. M. J. Mendez, et al., Functional transplant of megabase human immunoglobulin loci recapitulates human antibody response in mice. Nat. Genet. 15, 146-156 (1997).
30. K. Otero, et al., Macrophage colony-stimulating factor induces the proliferation and survival of macrophages via a pathway involving DAP12 and D-catenin. Nat. Immunol. 10, 734-743 (2009).
31. T. S. P. Heng, et al., The Immunological Genome Project: networks of gene expression in immune cells. Nat. Immunol. 9, 1091-1094 (2008).
32. D. Gate, et al., Clonally expanded CD8 T cells patrol the cerebrospinal fluid in Alzheimer's disease. Nature 577, 399-404 (2020).
33. A. Scialdone, et al., Computational assignment of cell-cycle stage from single-cell transcriptome data. Methods 85, 54-61 (2015).
34. X. Xu, et al., IFN-Stimulated Gene LY6E in Monocytes Regulates the CD14/TLR4 Pathway but Inadequately Restrains the Hyperactivation of Monocytes during Chronic HIV-1 Infection. J. Immunol. 193, 4125-4136 (2014).
35. H. Mathys, et al., Temporal Tracking of Microglia Activation in Neurodegeneration at Single-Cell Resolution. Cell Rep. 21, 366-380 (2017).
36. K. Van den Berge, et al., Trajectory-based differential expression analysis for single-cell sequencing data. Nat. Commun. 11, 1-13 (2020).
37. K. Kurakula, D. S. Koenis, C. M. van Tiel, C. J. M. de Vries, NR4A nuclear receptors are orphans but not lonesome. Biochim. Biophys. Acta-Mol. Cell Res. 1843, 2543-2555 (2014).
38. R. N. Hanna, et al., The transcription factor NR4A1 (Nur77) controls bone marrow differentiation and the survival of Ly6C-monocytes. Nat. Immunol. 12, 778-785 (2011).
39. N. Ipseiz, et al., The Nuclear Receptor Nr4a1 Mediates Anti-Inflammatory Effects of Aβ optotic Cells. J. Immunol. 192, 4852-4858 (2014).
40. C. K. Glass, K. Saijo, Nuclear receptor transrepression pathways that regulate inflammation in macrophages and T cells. Nat. Rev. Immunol. 10, 365-376 (2010).
41. R. D. Terry, et al., Physical basis of cognitive alterations in alzheimer's disease: Synapse loss is the major correlate of cognitive impairment. Ann. Neurol. 30, 572-580 (1991).
42. K. M. Rodrigue, K. M. Kennedy, D. C. Park, Beta-amyloid deposition and the aging brain. Neuropsychol. Rev. 19, 436-450 (2009).
43. T. K. Ulland, et al., TREM2 Maintains Microglial Metabolic Fitness in Alzheimer's Disease. Cell 170, 649-663.e13 (2017).
44. L. L. Green, et al., Antigen-specific human monoclonal antibodies from mice engineered with human Ig heavy and light chain YACs. Nat. Genet. 7, 13-21 (1994).
45. W. Song, et al., Alzheimer's disease-associated TREM2 variants exhibit either decreased or increased ligand-dependent activation. Alzheimer's Dement. 13, 381-387 (2017).

### 7.1.7. Methods

### Animals

TREM2^{CV}-5XFAD and TREM2^{R47H}-5XFAD mice were generated as previously described (19). Mice were housed in the animal facilities of Washington University in St. Louis. All animal experiments were conducted in compliance with Institutional regulations, under authorized protocols #20160220 and 19-0981 approved by the Institutional Animal Care and Use Committee of Washington University and Amgen South San Francisco. Only male mice were used for pharmacokinetic and pharmacodynamic analysis and scRNA-seq analysis was performed on both male and female mice in this study.

### Generation of Fully Human Anti-hTREM2 Antibodies

Fully human (hT2AB) and murinized (mT2AB) antibodies to hTREM2 were generated by gene-gun immunization of XMG2-K and XMG2-KL XenoMouse® transgenic mice (29, 44) with a cDNA encoding human TREM2 and DAP12. Antibody selection and generation was performed as described in SI Methods.

### Syk Phosphorylation Assay to Assess TREM2 Activation

A HEK293-based stable cell line expressing hTREM2 and hDAP12 (clone G13) or hMacs were used for antibody activity assessment as described in SI Methods. The antibody activity (stimulation) was reported as fold of control (S/B): S/B=Sample pSyk signal (counts)/Basal pSyk signal (isotype control pSyk signal counts). The EC₅₀ of hT2AB was determined by a four-parameter logistic fit model of GraphPad Prism Version 6.07.

Measurement of sTREM2 and CCL4 Levels in hMacs by MSD

hMacs were used for measuring the CCL4 and sTREM2 in conditioned media after treatment with hT2AB or hIgG1 isotype control antibody as described in SI Methods. Acetylated LDL was used as a positive control for each group of CCL4 measurement.

### Survival Assay of BMMs

BMMs from TREM2^{CV}, TREM2^{R47H} and Trem2^{-/-} were harvested at day 5 of culture with CSF1 and transferred to 24-well flat-bottom that were plated-bound hT2AB or control hIgG1 at 5×10⁴ cells/well in complete RPMI without CSF1. Survival, measured as % of PI negative cell population, was detected after 48 hours culture by a FACSCalibur.

### GFP Reporter Assay

2B4 NFAT:GFP reporter cells expressing hTREM2^{CV} and hTREM2^{R47H} have been described (45) and were used to test the activation of hTREM2 variants as described in SI Methods.

Pharmacodynamics and Pharmacokinetics of hT2AB

Pharmacodynamic analysis of hT2AB was performed as described in SI Methods. For pharmacokinetic analysis, groups of 8-month old TREM2^{CV}, TREM2^{R47H} and Trem2^{-/-} male or female mice were injected i.p. with a single injection of 30 mg/kg hT2AB. Concentrations of hT2AB in mouse serum samples and in homogenate of cold PBS-perfused cerebellum were measured 48 hours later with two different assays. Both assays were sandwich immunoassays, using a recombinant human TREM2 (Amgen, Inc. CA) and a ruthenium conjugated mouse anti-human Fc monoclonal antibody (Amgen, Inc. CA). The Lower Limit of Quantification (LLOQ) for serum and cerebellum homogenate assays were 100 ng/mL and 1 ng/mL, respectively.

### Single-Cell RNA-Seq

Eight-month old mice were injected i.p. with hT2AB or control hIgG1 at 30 mg/kg 48 hours before sacrifice. After perfusion with cold PBS, cortices were dissociated, and Cd45+ cells were sorted using FACS. All CD45+ cell libraries were prepared using the 10× Genomics Chromium Single Cell 3′ v2 Gene Expression Kit and sequenced on Illumina NovaSeq 6000 flow cells to achieve a read depth of 50,000 reads per cell. Data were analyzed as described in SI Methods. Samples that did not exhibit hT2AB exposure were excluded from downstream analyses. Sequencing data and sample quality reports can be obtained from Gene Expression Omnibus under series number GSE156183.

### Immunostaining for Aβ and Image Analyses

Free-floating brain sections were used for immunostaining of Aβ as described in SI Methods. The confocal pictures were taken on a Nikon A1Rsi+ confocal laser-scanning microscope using a 20×0.95-NA objective. z-Stacks with 1.1-μm steps in the z direction, 1,024×1,024-pixel resolution, were recorded. The percentage of Aβ area coverage was calculated automatically by batch processing in ImageJ.

### Aβ and Chemokine/Cytokines Quantification

Human Aβ₁₋₄₀ and Aβ₁₋₄₂ were measured by MSD V-plex 6E10 kit (Meso Scale Discovery) and mouse IL-1β, CXCL10 (IP-10) and CCL4 (MIP-1β) were measured by MSD U-plex biomarker group 1 Assay (Meso Scale Discovery) as described in SI Methods.

### Statistical Analyses

All graphs represent the mean of all samples in each group±SD or SEM as indicated in the figure legends. GraphPad Prism software (v8) was used to perform statistical analyses. P<0.05 was considered significant difference between different treatment groups, determining by two-way ANOVA with Sidak's multiple comparisons test, unless otherwise indicated.

### 7.1.8. Supplementary Methods (SI Methods)

### Animals

Mice were housed in the animal facilities of Washington University in St. Louis. All animal experiments were conducted in compliance with Institutional regulations, under authorized protocols #20160220 and 19-0981 approved by the Institutional Animal Care and Use Committee of Washington University and Amgen South San Francisco. Only male mice were used for pharmacodynamic analysis, pharmacokinetic analysis and scRNA-seq analysis were performed on both male and female mice in this study.

### Anti-hTREM2 Antibodies

hTREM2-specific serum titers obtained from immunized mice were monitored by live-cell FACS analysis (Accuri FACS). Lymphocytes from draining lymph nodes of animals with the highest antigen-specific serum native titers directed against hTREM2 were used for hybridoma generation. Hybridoma supernatants were screened for binding to human TREM2 by ELISA using 384-well plates coated with Neutravidin overnight or coated with control hIgG1 at 2 pg/mL at 37° C. for 1 hour followed by coating with biotinylated-hTREM2 extracellular domain fused to the Fc portion of hIgG1 Fc (hTREM2-Fc, Amgen). After a wash step, the exhausted hybridoma supernatants were diluted with 1% milk/1×PBS (1:5) and added to hTREM2-Fc or control hIgG1 coated 384 well plates and incubated at room temperature for 1 hour. A mixture of goat α-human κ-HRP (2060-05, Southern Biotech) and goat α-human λ-HRP (2070-05, Southern Biotech) were used for detection. The variable heavy and light chain sequences from a lead candidate identified in the hybridoma screening campaign were cloned and recombinantly expressed with an hIgG1 constant region lacking effector function to generate hT2AB. A murinized version of hT2AB, mT2AB, was generated by grafting the hT2AB variable domains on an effectorless mIgG1 backbone. Preparations of hT2AB, mT2AB, and the effectorless isotype control hIgG1 and control mIgG1 used in animal and cell-based experiments were tested for endotoxin and found to be comparable with <0.5 EU/mg assuring that responses were not due to TLR signaling.

### Antibody Binding Assay

The purified hT2AB was diluted to 5 pg/ml in assay buffer (10 mM Tris, 0.13% Triton X-100, 150 mM NaCl, 1 mM CaCl2), 0.1 mg/ml BSA, pH 7.6) and captured on anti-hFc kinetic sensors (18-5090, ForteBio). Recombinant hTREM1:GSS: Flag:6×His and recombinant hTREM2:GSS:Flag:6×His were minimally biotinylated (0.3-0.4 biotin/mol) and immobilized at 70-80 nM onto high precision Streptavidin fiber optic biosensors (SAX, #18-5119) over 2000 seconds to a final loading level of ˜2 nm. Each loaded TREM2 protein was then incubated with a dilution series of hT2AB Fab protein (30, 10, 3.3 nM) for 300 seconds and then 500 seconds in buffer alone (dissociation). Raw data was processed with the Octet data analysis software (v10) and processed data were globally fit to a 1:1 binding model and a dissociation constant (KD) of 50 nM was calculated. An anti-hTREM1 antibody and an isotype-matched hIgG2 control antibody were used as controls. To test the binding capacity to the hTREM2R47H mutation, bone marrow-derived macrophages (BMMs) from TREM2^{CV}, TREM2^{R47H} and Trem2^{-/-} mice were harvested on day 5 and incubated in FACS buffer (10% FCS in PBS) with hT2AB or control hIgG1 for 30 min, followed by staining with anti-hIgG Fc-PE (9040-09, SouthernBiotech). Dead cells were excluded by DAPI staining.

### Generation of Differentiated Human Monocyte-Derived Macrophages

Large, single donor lots (50-100 million cells per differentiation run) of cryopreserved CD14+ monocytes from healthy human de-identified donors were collected through leukapheresis and negative immunomagnetic selection (Lonza) and used to generate macrophages (hMacs). Suspensions of cryo-recovered monocytes were differentiated in RPMI-1640 medium using plant-derived recombinant M-CSF (50 ng/mL, plant-derived, ultra-low endotoxin 0.05 EU/pg, PromoCell #C-60442A) in a semi-adherent manner with CellGenix VueLife 118-C bio-process bags (Saint-Gobain Performance Plastics). A maximum of 50 million cells were loaded in differentiation medium into each bag (˜30 mL of cell suspension initial loading). Differentiation medium was composed of RPMI-1640+10% FBS (Gibco PerformancePlus Certified, heat inactivated, ≤5 EU/mL endotoxin, #10082139), 1× GlutaMAX (Gibco #35050061), 1× Pen/Strep (Gibco #15140122), 1×NEAA (Gibco #11140050), and 1× Sodium Pyruvate (Gibco #11360070) in addition to the 50 ng/mL M-CSF. With bags placed on racks in standard tissue culture incubators (humidified, 5% CO2, 37 degrees C.) to maximize gas exchange, differentiation was conducted for 9 days total with infusions of fresh differentiation medium on day 3 and day 6. After 9 days of differentiation, macrophages were collected from bio-process bags after agitation to dislodge cells and cryopreserved in BamBanker Serum Free Medium (Wako Chemicals USA #30214681). Each large-scale production run of human macrophages was qualified for consistent TREM2 expression relative to undifferentiated monocytes by flow cytometry. Throughout the production process, every effort was made to monitor and minimize endotoxin exposure.

### Syk Phosphorylation Assay

Clone G13 cells (8×105 cell/mL) or hMacs (2×105 cell/mL) were seeded overnight at 25 μL per well in a 384-well PDL-coated assay plate. On the day of the assay, cells were incubated with a serial dilution of hT2AB for 45-60 min at RT. After removal of the supernatant, the cells were lysed with M-Per+ containing 0.0625 nM anti-pSyk (Tyr525/526, 2710, Cell Signaling Technology) and 0.5 nM biotinylated mouse anti-Syk antibodies, Custom order: BD Bioscienes) at room temperature for 1 hour incubated with 2.5 pg/mL anti-rabbit IgG-Acceptor beads (AL104C, Perkin Elmer) at room temperature for 2 hours followed by addition of 10 pg/mL streptavidin-Donor beads (6760002B, Perkin Elmer) at room temperature for 2 hours. The antibody and bead concentrations stated are final. The AlphaLISA signals (counts) were measured by an EnVision Multilabel Reader.

Measurement of sTREM2 and CCL4 Levels in hMacs by MSD

hMacs were used for measuring the CCL4 and sTREM2 in conditioned media after treatment with hT2AB or hIgG1 isotype control antibody. Briefly, hMacs (500000 cells/well/ml) were plated in 6-well plates and incubated overnight at 37° C. Growth media was replaced by culture media (RPMI+GlutaMax+1% FBS) for 24 hours and the following day an appropriate amount of media was removed and replaced with media that contains hT2AB, hIgG1 isotype control antibody or acetylated LDL at a final concentration of 200 nM. At specified time points (4, 8 or 24 hours) media from each treated well (conditioned media) was removed and saved for analysis until all samples were collected. CCL4 (4, 8 or 24 hours) and sTREM2 (24 hours) levels were measured in conditioned media with an MSD platform-based assay as per manufacturer instructions (Meso Scale Discovery).

### Survival Assay of BMMs

BMMs from TREM2^{CV}, TREM2^{R47H} and Trem2 were harvested at day 5 of culture with CSF1 and transferred to 24-well flat-bottomed plates that were coated with hT2AB or control hIgG1 at 5×104 cells/well in complete RPMI without CSF1. Survival, measured as % of Propidium Iodide negative cell population, was detected after 48 hours culture by a FACSCalibur.

### GFP Reporter Assay

2B4 NFAT:GFP reporter cells expressing hTREM2CV and hTREM2R47H were used to test the activation of hTREM2 variants. Briefly, stock solutions of hT2AB or control hIgG1 were diluted to the indicated concentrations in a sodium carbonate-bicarbonate buffer, and 50 μl of the resulting solution was added to distinct wells of a 96-well plate for overnight. Each condition was performed in triplicate. The antibody coated plates were washed three times by cold PBS before transferring 2B4 NFAT:GFP reporter cells expressing hTREM2CV or hTREM2R47H into the corresponding wells at 1000 cells/μl. After stimulating for 16 hours, the cells were transferred to FACS tubes and read on a FACSCalibur for GFP expression.

Pharmacodynamics and Pharmacokinetics of hT2AB

Groups of 10-week old TREM2^{CV}, TREM2^{R47H} and Trem2^{-/-} male mice were injected intravenously (i.v.) with different doses of hT2AB. After 48 hours, mice were sacrificed and brain lysates were used to measure the concentrations of CXCL10, CCL4, CCL2, CXCL2 and CST7 by MSD. In a different treatment group, TREM2R47H and Trem2−/− male mice were injected i.v. with hT2AB at 30 mg/kg. Mice were sacrificed at 4, 8 and 24 hours after injection. The relative gene expression levels of Cxcl10, Ccl2, Ccl4, Cst7 and Tmem119 were measured by qRT-PCR. For pharmacokinetic analysis, groups of 8-month old TREM2^{CV}, TREM2^{R47H} and Trem2^{-/-} male or female mice were injected intraperitoneally with a single injection of 30 mg/kg hT2AB. Concentrations of hT2AB in mouse serum samples and in homogenate of cold PBS-perfused cerebellum were measured 48 hours later with two different assays. Both assays were sandwich immunoassays, using a recombinant human TREM2 (Amgen, Inc. CA) and a ruthenium conjugated mouse anti-human Fc monoclonal antibody (Amgen, Inc. CA). The Lower Limit of Quantification (LLOQ) for serum and cerebellum homogenate assays were 100 ng/mL and 1 ng/mL, respectively.

### Single-Cell RNA-Seq Analyses

### Computational Resources

Computational resources were readily available online from their respective websites.

| Resource | Version | Resource | Version |
|---|---|---|---|
| Cell Ranger | 3.0.2 | R Statistical Software | 3.60 |
| PANTHER | 15.0 | irlba | 2.3.3 |
| Python | 3.7.4 | scran | 1.14.5 |
| umap-learn | 0.3.10 | batchelor | 1.2.4 |
| Java | 1.8.0 | dbscan | 1.1.5 |
| Leiden | 1.0.0 | SingleR | 1.0.5 |
| | | igraph | 1.2.4.2 |
| | | caret | 6.0.86 |
| | | singshot | 1.4.0 |
| | | tradeSeq | 1.0.1 |

### Read Alignment

A reference genome was built by extending mouse mm10 by human TREM2 from GRCh38; transcriptome annotations from Ensembl 93 were used. Sequenced reads from the microfluidic droplet platform were de-multiplexed and aligned using CellRanger version 3.0.2, available from 10x Genomics (www.10xgenomics.com), with default parameters. Quality control We performed a multi-step quality assessment. First, each sample was analyzed individually (FIG. S1A). Here, all 24 samples passed an initial quality control evaluating the distribution of all mapped reads over the genome and the fraction of sequenced bases with a Phred quality score (Q)>30. Each sample encompasses thousands of captured events, k, which are either genuine cells or empty droplets with ambient RNA; each event is identified by a unique barcode b. To distinguish single cells, a method based on the conventional thresholding on the total UMI count was used, u=(u_{b})∈ [symbol] .

First, each barcode b meeting

$${u}_{b} > {\sum }_{b = 1}^{k} \frac{{u}_{b}}{k}$$

k was rank transformed in order of decreasing number of total UMI count resulting in a vector r=(r_{b})∈ [symbol] ; typically

$$\sum \frac{{u}_{b}}{k} \approx 72.$$

For barcodes with equal UMI counts, a permutation was used with increasing values at each index set of ties. The total UMI count was then modeled as a function of barcode rank, ln (u) ˜ln (r), by fitting cubic smooth splines with 20 degrees of freedom. Each inflection point of this function may be interpreted as a transition between a subset of barcodes with a larger number of total UMIs, i.e., potentially cell-containing droplets, and the majority of barcodes with ambient RNA. Inflection points were determined by local minima of the first differentiation of the spline basis functions. For each sample, the inflection point φ closest to the expected number of recovered cells was chosen and all barcodes meeting u_{b}>φ were retained. The choice of φ was further guided by the following descriptive metrics of the selected set of barcodes: i) percentage of all reads allotted to the selected barcodes, ii) median number of reads per barcode, iii) median fraction of reads mapped to mitochondrial genes per barcode, iv) median number of UMI per barcode, v) median number of genes with at least one UMI count per barcode, and vi) median fraction of reads originating from an already-observed UMI (saturation). The cell selection was further refined by evaluating the distributions of metrics ii-vi) to remove low quality cells and doublets. Finally, we estimated the cell cycle effect in each sample. The cell cycle phase per cell was predicted using the machine learning based approach proposed by Scialdone et al. (1). Briefly, a classifier was trained on pairs of genes that change expression directionality across cell cycle phases. Each cell's cell cycle state can then be projected by examining the sign of the expression difference in the new data set. Cells with a predicted G1 or G2M score above 0.5 were assigned to the G1 or G2M phases, respectively; cells were classified to be in S phase, if the predicted G1 and G2M scores were below 0.5. All calculations were performed using the cyclone function in the R package scran. Predicted cell cycle scores and phases were not used for cell filtering.

Next, an integrative quality control step was conducted to identify unwanted technical artifacts in the data. All filtered 94488 cells from all samples were pooled, and the resulting UMI count matrix was normalized and log-transformed (see Normalization). To reveal technical artifacts in the data, principal components were calculated that capture the maximal variance within the data while controlling for the manifest variables sex, age, and treatment. For this purpose, manifest variables were partialled-out from the normalized UMI count matrix. Informative genes were unbiasedly determined by their mean expression-dependent variance in the data (see Gene expression variance modeling) and used for calculating principal components of the corrected UMI count matrix (see Spectral dimensionality reduction). The resulting 38-dimensional latent space was further modeled with a fuzzy topological structure using Uniform Manifold Approximation and Projection (UMAP; Python package umap-learn) (2) to unfold the data structure that is either driven by cell type or technical variance. While varying library sizes can be normalized between cells, a large fraction of missing values (i.e., drop-outs) due to poor transcriptome coverage cannot be accurately recovered in the data and will significantly impact downstream analyses. Thus, to score cell quality, the first principal component of a set of seven quality metrics assessing the transcriptome coverage per cell was calculated: i-iv) fraction of reads consumed by the top {500, 200, 100, 50} expressed genes, v) fraction of mitochondrial reads, vi) relative distance to the maximum total number of UMIs (i.e.,

$$1 - \frac{{u}_{b}}{max ( u )} ) ,$$

vii) relative distance to the maximum number of genes with at least one UMI count. The resulting cell quality (CQ) score was then superimposed onto the UMAP revealing a technical confounder in the data (FIG. S1B). Using density-based spatial clustering (R package dbscan), we extracted a representative group of cells with low CQ score. We determined an optimal CQ score cut-off of −0.1 which removes a maximum fraction of cells within this group (91.4%) and a minimum fraction of cells otherwise (11.1%) by determining the knee of the inverse empirical cumulative CQ score distribution function (FIG. S1C). This resulted in a final data set of 71303 high quality single cells (FIG. SID).

### Normalization

Library sizes were normalized as proposed by Lun et al. (3). In short, size factors were computed from pools of similar cells, which were then deconvoluted to cell-based factors and used to scale the counts in each cell. First, for each batch scaling factors for each cell were calculated using the R package scran. An initial guess of cell populations contained in the data was calculated by using the function quickCluster on a shared nearest neighbor graph; we required a minimum cluster size φ of 10% of the total number of cells, and a minimum average gene expression of 1 for the shared nearest neighbor graph construction. Size factors were then calculated using the function computeSumFactors with pool sizes ranging in [21, max{101, φ+1}∈ [symbol] . Then scaling factors were normalized between batches based on their ratio of average UMI counts to provide comparable results to the lowest-coverage batch using the R package batchelor. Finally, the scaled UMI count matrix X was log 2 transformed by log₂(X+1).

### Supervised CD45+ Cell Type Annotation

Rather than determining cell identities by a biased selection of marker genes, cell types were unbiasedly characterized using the total available transcriptome. For this purpose, robustly expressed genes with more than 4 molecules in at least 50 cells were first filtered, ribosomal and mitochondrial genes were removed (obtained from Gene Ontology GO:0005840 and Ensembl, respectively), only protein-coding genes were retained, and a set of 136 genes highly correlating with dissociation-induced stress response (4) were removed. This resulted in a single-cell expression matrix X composed of n data vectors xⱼ with j∈1, . . . , 71303 of dimensionality m=4453: X=(xᵢⱼ)∈ [symbol] . Log-normalized microarray gene expression data of 20 immune cell types measured in 830 pure samples of sorted cells by the Immunological Genome Project (ImmGen) (5) were used; the processed expression matrix X=(xᵢⱼ)∈ [symbol] was obtained from the SingleR R package. 3697 genes were filtered that were highly variable in this dataset by assessing the mean expression-dependent variance (see Gene expression variance modeling). Spearman's rank correlation coefficients, ρ(xᵢ, yⱼ), were calculated between all single cells, xᵢ ∈ [symbol] , and all ImmGen samples, yⱼ∈ [symbol] , using 992 genes overlapping between both datasets. Each single cell was assigned the cell type with the most similar expression profile by arg maxⱼ ρ(xᵢ, yⱼ). In this step 15 cell types were detected in the dataset. Since the original expression space of single-cell data is rife with noise and the underlying distributions of scRNA-seq UMI counts and microarray signal intensities differ, we aimed to refine the initial cell type classification. For this purpose, the manifest variables sex, age, and treatment, as well as, three technical confounders (fraction of reads mapped to mitochondrial genes, total number of reads, fraction of reads assigned to dissociation-related stress response genes) were regressed-out from X. Then, a 39-dimensional principal component space was calculated (see Spectral dimensionality reduction) on highly variable genes (see Gene expression variance modeling) and subjected to UMAP (Python package umap-learn) to unfold the data structure driven by cell identity. Next, we generated a weighted cell adjacency matrix; weights were calculated by the Jaccard index between cells using the overlap of their 15-nearest neighborhood. We identified 30 cell communities (segments) in the adjacency matrix using the Leiden algorithm (6) (Java package Leiden) with a resolution of 3×10-4. The data was then tabulated by counting for each segment i the number of cells with cell type j resulting in matrix A=(aᵢⱼ)∈ [symbol] . To avoid division by 0 in the subsequent calculation, a pseudocount was added by A=A+1. An enrichment score matrix A′=(a′ᵢⱼ)∈ [symbol] was derived by

$${a}_{ij}^{′} = ln ( \frac{{a}_{ij}}{{\sum }_{j = 1}^{15} {a}_{ij} - {a}_{ij}} ) - ln ( \frac{{\sum }_{i = 1}^{30} {a}_{ij} - {a}_{ij}}{{\sum }_{i = 1}^{30} {\sum }_{j = 1}^{15} {a}_{ij} - {\sum }_{j = 1}^{15} {a}_{ij} - {\sum }_{i = 1}^{30} {a}_{ij} + {a}_{ij}} )$$

Finally, each cell in each segment was assigned the cell type with the highest enrichment score. This revealed 64274 microglia cells in the dataset.

### Differential Gene Expression Analysis

To identify differentially expressed genes between two groups of cells, we developed an intuitive and scalable approach based on Bayesian statistics. Here, we calculated the specificity and the detection rate for each gene in each group. We defined specificity by the posterior probability P(j=Z|xᵢⱼ>φ). It defines the probability that cell j is a member of group Z if feature i was observed higher expressed than a threshold φ. Detection level is defined by the posterior probability P(xᵢⱼ>φ|j=Z), i.e., the relative fraction of cells expressing a gene i above threshold φ in group Z. The threshold parameter φ was set to 2 (i.e. at least 4 molecules per cell). All conditional probabilities were calculated using Bayes' theorem with adjusted marginal probabilities to avoid sample size bias:

$$P ( j = Z ) = 0.5$$

$$P ( {x}_{ij} > φ ) = \frac{P ( {x}_{ij} > φ \vert j = Z ) λ + P ( {x}_{ij} > φ \vert j \neq Z ) λ}{2 λ}$$

with λ is the maximum cardinality of both groups. In addition, absolute differences between expression intensity means of gene i were estimated using the effect size defined by Cohen's d:

$$d ( {x}_{1.} , {x}_{2.} ) = \frac{{μ}_{1} - {μ}_{2}}{{σ}_{1 , 2}}$$

with μ is the gene expression mean and σ_{1,2} is the pooled standard deviation of two samples {1,2}:

$${σ}_{1 , 2} = \sqrt{\frac{( {n}_{1} - 1 ) {σ}_{1}^{2} + ( {n}_{2} - 1 ) {σ}_{2}^{2}}{{n}_{1} + {n}_{2} - 2}}$$

with σ² is the gene expression variance in one group.

### Microglia Baseline Annotation

For subsequent downstream analyses, we filtered the normalized and processed gene expression matrix X by robustly expressed genes with more than 2 molecules in at least 50 cells, removed ribosomal and mitochondrial genes (obtained from Gene Ontology GO:0005840 and Ensembl, respectively), retained only protein-coding genes, and removed a set of genes highly correlating with dissociation-induced stress response (4). To annotate the microglia baseline cell heterogeneity, we extracted 5694 cells from male and female control hIgG1 treated TREM2^{CV}-5XFAD mice. This resulted in the filtered single-cell expression matrix X′=(x′ᵢⱼ)∈ [symbol] . The data vectors of highly variable genes (see Gene expression variance modeling) were embedded [symbol] proximal to a non-linear lower dimensional manifold using Diffusion Maps (see Spectral dimensionality reduction). This revealed a temporal axis in the data, presumably a microglia activation trajectory. A weighted cell adjacency matrix was calculated from the diffusion components by using the Jaccard index of the overlap of each cell's nearest neighborhoods. The resulting graph was clustered using Louvain's community detection method available in the R package igraph. To better understand the underlying temporal topology of the data, we fitted an unconstrained maximum parsimony tree between all 11 clusters using the getLinages function from the slingshot R package. It revealed a branching trajectory with 5 terminal ends. The cluster with the highest expression of common microglia marker was determined to be the resting microglia population. We further compared our clusters to a reference expression profile of DAMs from a previous study (7). First, we calculated vectors with gene expression log 2 fold-changes between all clusters and the resting microglia population in our data. Then, we computed a single vector of log 2 fold-changes between stage 2 DAMs and the resting population in the reference study. Overall, 10124 genes overlapped between both studies, resulting in a log 2 fold-change vector zᵢ ∈ [symbol] for each cluster and the reference. Only genes with an absolute log 2 fold-change of >0.5 were considered differentially expressed and retained for the subsequent analysis. Each log 2-fold change vector was binarized by using the signum function sign(zᵢ). We tabulated the data by counting the agreements and disagreements in expression directionality between our data and the reference study resulting in a contingency table, A=(aᵢⱼ)∈ [symbol] . We interpreted this table as confusion matrix, i.e., how well our data predicts the expression trend of the refence DAM population. Here, we calculated a similarity score by subtracting the sum of the off-diagonal values (false positives+false negatives) from the matrix trace (true positives+true negatives). We further tested the null hypothesis that the overall agreement between both datasets defined by trace(A)/ΣᵢΣⱼaᵢⱼ is less or equal than the no-information rate defined by the fraction of the largest class in the data; test statistics were calculated using the R package caret.

The predicted cell cycle phases of each cell were used to identify a cluster of cycling microglia cells; cell cycle phase prediction and scoring were performed in the quality control step (see Quality control). Gene Ontology term enrichment analyses were performed using the PANTHER classification system (8) to functionally characterize the expression profile of selected clusters. Marker genes were determined by contrasting one cluster against a cell pool of all other clusters (see Differential gene expression analysis); genes were selected by meeting minimum specificity/detection rate/effect size thresholds (IFN-R: 75.0%/10.0%/1.5, MHC-II: 50.0%/10.0%/1.0). We used the 11361 genes contained in our dataset as reference list for each statistical overrepresentation test. False discovery rate was used to correct Fisher's exact test P-values for multiple testing.

### Sample Harmonization

Given that hT2AB and control hIgG1 injections were performed in mice of different sex and carrying distinct TREM2 variants, we next developed a supervised sample harmonization approach to define the impact of sex, genotype, and treatment on the four microglial trajectories (FIG. S3A). We used our results from the baseline analysis of microglia control hIgG1-treated mice as a reference for classifying cells of samples from different conditions. To minimize classification errors, diffusion components of the reference dataset and each query dataset were calculated (see Spectral dimensionality reduction). Machine learning was applied on the lower-dimensional manifold to learn cell types from the reference set and to classify the query dataset using Xtreme gradient boosting. Hyperparameters of the machine learning model were optimize using grid search provided by the R package caret. Since the manifold is describing a developmental continuum with overlapping cell cluster boundaries, we accepted a reasonable average training accuracy of 87.5% to avoid overfitting. To assess overall classification accuracy, we used reverse projection. We trained a classifier on the predicted classes of the query dataset and projected the cell types for the reference dataset. An average training accuracy of 92.9% indicates that the predicted classes of the query dataset are highly coherent. Our model achieved a high overall average prediction accuracy of 86.3% (FIG. S3B).

### Microglial Cell Type Trajectory Reconstruction

To analyze distinct trajectories for each microglia cell type, we extracted the branching cluster, all terminal clusters, and the intermediate cluster t6 from the expression matrix [symbol] containing all samples. The data was then split by cell type: DAM trajectory={t5, t6, DAM}, Cyc-M trajectory={t5, Cyc-M}, IFN-R trajectory={t5, IFN-R}, MHC-II trajectory={t5, MHC-JI}. Cells of each cell type trajectory were embedded onto a lower-dimensional manifold using diffusion maps (see Spectral dimensionality reduction) on the highest variable genes (see Gene expression variance modeling) and projected onto 2-dimensions with UMAP to unfold the latent temporal axis. To further order cells chronologically, we fitted smooth trajectory curves for each linage using principal curves and projected datapoints orthogonally using the R package slingshot; here, arc lengths were interpreted as pseudotime values (9).

### Cell Type Compositional Analysis

We aimed to delineate differences in microglial cell-type proportions due to sex, genotype and treatment. Cell type fractions in the single-cell data are not linearly corresponding to true proportions in vivo due to technical artifacts, including cell damage induced by sample preparation and handling or by pressure changes during cell sorting, amongst others. Further, small sample sizes may not properly represent the actual population. This may cause large variances in cell type proportions between biological replicates. By using stratified bootstrap resampling we can estimate the population mean and correct the sampling bias of individual replicates, as well as, measure estimate uncertainty. Let W=(wᵢⱼ)∈ [symbol] a label matrix composed of 2-dimensional vectors with a label indicator (e.g., cell type or time interval) and a biological replicate indicator for m cells, l label levels, and d biological replicates. For example, w_{1.}={10, 2} is the label vector for the first cell listed in W, which was sampled from time interval 10 and from the second biological replicate. We constructed stratified resamples of size k, where k was set to the maximum replicate size. Each resample was then tabulated resulting in a matrix V=(vᵢⱼ)∈ [symbol] which contained absolute numbers of cells per cell label and replicate. To conservatively correct biases in single replicates, resamples were aggregated by

$$b = \frac{{min}_{i} {v}_{ij}}{k} , j = 1 , … l$$

The resulting vector b∈ [symbol] represented a sample of cell type proportions. We performed N=500 bootstrapping iterations resulting in a matrix B=(bᵢⱼ)∈ [symbol] . Estimates of cell-type proportions E [symbol] were derived by

$$μ = \frac{{\sum }_{i = 1}^{N} {b}_{ij}}{N} , j = 1 , … l$$

The standard error for the 95% confidence interval for an estimated cell type proportion of label j were derived by:

$${SE}_{j} = 1.96 \frac{{σ}_{j}}{\sqrt{N}} with {σ}_{j} = \sqrt{\frac{{\sum }_{i = 1}^{N} {b}_{ij} - {μ}_{j}}{N - 1}}$$

### Inference of Expression Dynamics

We fitted gene expression as a function of pseudotime for each microglia cell type trajectory on a subset of cells from a specified genotype, sex and treatment using negative binomial generalized additive models (NB-GAM) (10). Wald tests were performed to test the hypothesis if the beginning or the end of a gene's dynamic curve differs between conditions and corresponding log fold-changes were calculated using parameters of the NB-GAM smoothers. These computations were performed with the R package tradeSeq. To statistically classify gene dynamics, Wald test P-values were corrected for multiple testing via false discovery rate. Each corrected P-value p was weighted by the sign of the log fold-change S by p^{S} and −log 10 transformed. Resulting negative values denoted downregulation and positive values indicated upregulation, respectively.

### Gene Expression Variance Modeling

By assuming that biological heterogeneity is driven by a subset of genes having high variance between cells, we aim to improve resolution by removing genes driven by technical noise. Total variance of each gene is decomposed into its biological and technical components by fitting the variance as a function of mean expression (11). Significance is inferred by modeling the residuals of this fit with an F-distribution. To retain condition-specific variance, the union of all highly variable genes of all batches is always used. Variance decomposition and F-distribution statistics were calculated using the R package scran.

### Spectral Dimensionality Reduction

This step aims to reduce redundancy and to improve the signal-to-noise ratio in the data, which eventually will reveal latent biological factors in the data. For this purpose, we employed spectral dimensionality reduction methods. Linear spectral embedding is obtained by a Principal Component Analysis (PCA). This method captures the maximal variance in the data. It may miss substructures in the data but is sufficient for data with low intrinsic complexity or may be used to get a first insight into the data structure. PCA was calculated with the R package irlba. Non-linear embedding was performed by using Diffusion Maps (12). This method resolves non-linear and linear substructures in the data based on a cell dissimilarity matrix calculated from a distance function Don each cell's expression profile. However, instead of calculating the diffusion components using an estimated global sigma in the diffusion kernel K with

$$K ( {x}_{i} , {x}_{j} ) = exp ( \frac{- {D ( {x}_{i} , {x}_{j} )}^{2}}{2 σ} ) ,$$

multiple local sigmas were used, as proposed by Haghverdi et al. (13):

$$K ( {x}_{i} , {x}_{j} ) = \sqrt{\frac{2 {σ}_{i} {σ}_{j}}{{σ}_{i}^{2} + {σ}_{j}^{2}}} exp ( \frac{- {D ( {x}_{i} , {x}_{j} )}^{2}}{{σ}_{i}^{2} + {σ}_{j}^{2}} )$$

We account for batch effects in the data as follows. For PCA, we center the input expression matrix by the mean of the center vectors of each batch and scale the covariance matrix by the total cell count of each batch. This ensures that each batch contributes equally to the identification of the loading vectors (i.e., the PCA won't be dominated by samples with high cell count). For Diffusion Maps, we use the robust pairwise cosine correlation distance. For both techniques, the resulting lower-dimensional matrix was corrected for batch effects using mutual nearest neighbors' batch correction (14) (R package batchelor). The number of selected components was guided by a scree plot analysis.

### Immunostaining for Aβ and Image Analyses

Free-floating brain sections were initially blocked and permeabilized with PBS+3% BSA solution containing 0.25% Triton X-100. Primary antibodies were added at a dilution of 1:1,000 for Aβ1-16 (6E10, conjuncted Alexa Fluor 488, 803013, BioLegend), and 1:500 for Aβ42 (rabbit recombinant monoclonal, Thermo Fisher Scientific) at 4° C. for overnight. Secondary antibodies, anti-rabbit IgG Alexa Fluor 647 (goat recombinant polyclonal, 1:1,000; Invitrogen) and methoxy-X04 (3 pg/ml; Tocris) were added for 1.5 hours at room temperature. The confocal pictures were taken on a Nikon A1Rsi+ confocal laser-scanning microscope using a 20×0.95-NA objective. z-Stacks with 1.1-mm steps in the z direction, 1,024×1,024-pixel resolution, were recorded. The percentage of Aβ area coverage was calculated automatically by batch processing in ImageJ.

### Aβ and Chemokine Cytokines Quantification

Human Aβ₁₋₄₀ and Aβ₁₋₄₂ were measured by MSD V-plex 6E10 kit (Meso Scale Discovery) and mouse IL-lb, CXCL10 (IP-10) and CCL4 (MIP-lb) were measured by MSD U-plex biomarker group 1 Assay (Meso Scale Discovery). Briefly, cortical tissues from antibody or control hIgG1 treated mice were homogenized with Tissue Lyser II (Qiagen) in 12× v/w of PBS containing 0.5% of Triton x-100 and 1× Halt protease inhibitor cocktail. The insoluble fraction was pelleted by ultracentrifugation at 100,000 g for 1 hour. Supernatant was collected as soluble PBS fraction. The pellet was resuspended in 250 μl of 6 M guanidine and 50 mM Tris, pH 8.0, buffer, and was further homogenized by sonication, followed by ultracentrifugation at 75,000 rpm to clarify the denatured pellet. The supernatant was collected as the insoluble guanidine fraction.

### 7.1.9. Supplementary References

The specific references below, and discussed herein, are incorporated in their entirety.
1. A. Scialdone, et at, Computational assignment of cell-cycle stage from single-cell transcriptome data. Methods 85, 54-61 (2015).
2. E. Becht, et al., Dimensionality reduction for visualizing single-cell data using UMAP. Nat. Biotechnot 37, 38-47 (2019).
3. A. T. L. Lun, K. Bach, J. C. Marioni, Pooling across cells to normalize single-cell RNA sequencing data with many zero counts. Genome BioL 17, 1-14 (2016).
4. S. C. Van Den Brink, et al., Single-cell sequencing reveals dissociation-induced gene expression in tissue subpopulations. Nat. Methods 14, 935-936 (2017).
5. T. S. P. Heng, et al., The Immunological Genome Project: networks of gene expression in immune cells. Nat. Immunot 9, 1091-1094 (2008).
6. V. A. Traag, L. Waltman, N. J. van Eck, From Louvain to Leiden: guaranteeing well-connected communities. Bd. Rep. 9, 1-12 (2019).
7. H. Keren-Shaul, et al., A Unique Microglia Type Associated with Restricting Development of Alzheimer's Disease. Cell 169, 1276-1290.e17 (2017).
8. H. Mi, et al., Protocol Update for large-scale genome and gene function analysis with the PANTHER classification system (v.14.0). Nat. Protoc. 14, 703-721 (2019).
9. K. Street, et al., Slingshot: cell lineage and pseudotime inference for single-cell transcriptomics. BMC Genomics 19, 477 (2018).
10. K. Van den Berge, et al., Trajectory-based differential expression analysis for single-cell sequencing data. Nat. Commun. 11, 1-13 (2020).
11. A. T. L. Lun, D. J. Mccarthy, J. C. Marioni, A step-by-step workflow for low-level analysis of single-cell RNA-seq data with Bioconductor [version 2; referees: 3 approved, 2 approved with reservations]. F1000Research (2016).
12. R. R. Coifman, S. Lafon, Diffusion maps. Appl. Comput. Harmon. Anal. 21, 5-30 (2006).
13. L. Haghverdi, F. Buettner, F. J. Theis, Diffusion maps for high-dimensional single-cell analysis of differentiation data. Bioinformatics 31, 2989-2998 (2015).
14. L. Haghverdi, A. T. L. Lun, M. D. Morgan, J. C. Marioni, Batch effects in single-cell RNA-sequencing data are corrected by matching mutual nearest neighbors. Nat. Biotechnol. 36, 421-427 (2018).

We claim:

1. A method of identifying a patient with Alzheimer's disease who will benefit from treatment with a TREM2 agonist, comprising:
(a) obtaining a first biological sample from the patient prior to administration of the TREM2 agonist to the patient;
(b) measuring a level in the first biological sample of one or more biomarkers selected from APOE, B2M, BIRC5, BST2, C1QA/B/C, CCL12, CCL2, CCL3, CCL4, CCNB2, CD3G, CD63, CD74, CD81, CD9, CST7, CTSB, CXCL10, CXCL2, FTL1, H2-AA, H2-AB1, H2AFV, H2AFZ, H2-D1, H2-DMA, H2-DMB1, H2-DMB2, H2-EB1, H2-K1, H2-OA, H2-OB, H2-Q10, H2-Q4, H2-Q6, H2-Q7, H2-T23, HEXB, HMGB2, HMGN2, IFI204, IFI2712A, IFIT3, IFITM3, IL1B, IRF7, ISG15, LGALS3BP, LGMN, LPL, LY6E, MLF, MR1, MRC1, MS4A4B, OASL2, OLFML3, P2RY12, PF4, RTP4, SLFN2, SPARC, STMN1, TMEM119, TUBA1B, TUBB5, and USP18;
(c) administering to the patient an effective amount of a TREM2 agonist;
(d) obtaining a second biological sample from the patient after administration of the TREM2 agonist to the patient; and
(e) measuring a level in the second biological sample of one or more biomarkers selected from APOE, B2M, BIRC5, BST2, C1QA/B/C, CCL12, CCL2, CCL3, CCL4, CCNB2, CD3G, CD63, CD74, CD81, CD9, CST7, CTSB, CXCL10, CXCL2, FTL1, H2-AA, H2-AB1, H2AFV, H2AFZ, H2-D1, H2-DMA, H2-DMB1, H2-DMB2, H2-EB1, H2-K1, H2-OA, H2-OB, H2-Q10, H2-Q4, H2-Q6, H2-Q7, H2-T23, HEXB, HMGB2, HMGN2, IFI204, IFI2712A, IFIT3, IFITM3, IL1B, IRF7, ISG15, LGALS3BP, LGMN, LPL, LY6E, MLF, MR1, MRC1, MS4A4B, OASL2, OLFML3, P2RY12, PF4, RTP4, SLFN2, SPARC, STMN1, TMEM119, TUBA1B, TUBB5, and USP18.

2. A method of predicting a treatment response of Alzheimer's disease in a patient to a TREM2 agonist, comprising the steps of:
(a) obtaining a first biological sample from the patient prior to administration of the TREM2 agonist to the patient;
(b) measuring a level in the first biological sample of one or more biomarkers selected from APOE, B2M, BIRC5, BST2, C1QA/B/C, CCL12, CCL2, CCL3, CCL4, CCNB2, CD3G, CD63, CD74, CD81, CD9, CST7, CTSB, CXCL10, CXCL2, FTL1, H2-AA, H2-AB1, H2AFV, H2AFZ, H2-D1, H2-DMA, H2-DMB1, H2-DMB2, H2-EB1, H2-K1, H2-OA, H2-OB, H2-Q10, H2-Q4, H2-Q6, H2-Q7, H2-T23, HEXB, HMGB2, HMGN2, IFI204, IFI2712A, IFIT3, IFITM3, IL1B, IRF7, ISG15, LGALS3BP, LGMN, LPL, LY6E, MLF, MR1, MRC1, MS4A4B, OASL2, OLFML3, P2RY12, PF4, RTP4, SLFN2, SPARC, STMN1, TMEM119, TUBA1B, TUBB5, or USP18;
(c) treating the biological sample from the patient or a reference sample;
(d) measuring a level in the treated biological sample of one or more biomarkers selected from APOE, B2M, BIRC5, BST2, C1QA/B/C, CCL12, CCL2, CCL3, CCL4, CCNB2, CD3G, CD63, CD74, CD81, CD9, CST7, CTSB, CXCL10, CXCL2, FTL1, H2-AA, H2-AB1, H2AFV, H2AFZ, H2-D1, H2-DMA, H2-DMB1, H2-DMB2, H2-EB1, H2-K1, H2-OA, H2-OB, H2-Q10, H2-Q4, H2-Q6, H2-Q7, H2-T23, HEXB, HMGB2, HMGN2, IFI204, IFI2712A, IFIT3, IFITM3, IL1B, IRF7, ISG15, LGALS3BP, LGMN, LPL, LY6E, MLF, MR1, MRC1, MS4A4B, OASL2, OLFML3, P2RY12, PF4, RTP4, SLFN2, SPARC, STMN1, TMEM119, TUBA1B, TUBB5, or USP18;
(e) comparing one of more biomarkers in the pre-treatment biological sample with one or more biomarkers in the treated biological sample or treated reference sample; and
(f) optionally, proceeding with administration of the TREM2 agonist to the patient, if such administration is predicted to have an equivalent or higher likelihood of success relative to an alternative method of treating the Alzheimer's disease;
(g) wherein the biomarker change in response to step (c) is predictive of the likelihood of successful treatment of the Alzheimer's disease based on a greater or lesser biomarker change compared with one or more similar patients and as evaluated using one or more of the biomarkers.

3. A method of treating Alzheimer's disease with a TREM2 agonist, comprising:
(a) obtaining a first biological sample from the patient prior to administration of the TREM2 agonist to the patient;
(b) measuring a level in the first biological sample of one or more biomarkers selected from APOE, B2M, BIRC5, BST2, C1QA/B/C, CCL12, CCL2, CCL3, CCL4, CCNB2, CD3G, CD63, CD74, CD81, CD9, CST7, CTSB, CXCL10, CXCL2, FTL1, H2-AA, H2-AB1, H2AFV, H2AFZ, H2-D1, H2-DMA, H2-DMB1, H2-DMB2, H2-EB1, H2-K1, H2-OA, H2-OB, H2-Q10, H2-Q4, H2-Q6, H2-Q7, H2-T23, HEXB, HMGB2, HMGN2, IFI204, IFI2712A, IFIT3, IFITM3, IL1B, IRF7, ISG15, LGALS3BP, LGMN, LPL, LY6E, MLF, MR1, MRC1, MS4A4B, OASL2, OLFML3, P2RY12, PF4, RTP4, SLFN2, SPARC, STMN1, TMEM119, TUBA1B, TUBB5, or USP18;
(c) administering to the patient an effective amount of a TREM2 agonist;
(d) obtaining a second biological sample after administration of the TREM2 agonist to the patient; and
(e) measuring a level in the second biological sample of one or more biomarkers selected from APOE, B2M, BIRC5, BST2, C1QA/B/C, CCL12, CCL2, CCL3, CCL4, CCNB2, CD3G, CD63, CD74, CD81, CD9, CST7, CTSB, CXCL10, CXCL2, FTL1, H2-AA, H2-AB1, H2AFV, H2AFZ, H2-D1, H2-DMA, H2-DMB1, H2-DMB2, H2-EB1, H2-K1, H2-OA, H2-OB, H2-Q10, H2-Q4, H2-Q6, H2-Q7, H2-T23, HEXB, HMGB2, HMGN2, IFI204, IFI2712A, IFIT3, IFITM3, IL1B, IRF7, ISG15, LGALS3BP, LGMN, LPL, LY6E, MLF, MR1, MRC1, MS4A4B, OASL2, OLFML3, P2RY12, PF4, RTP4, SLFN2, SPARC, STMN1, TMEM119, TUBA1B, TUBB5, or USP18;
wherein when the level of one or more biomarkers selected from APOE, B2M, BIRC5, BST2, C1QA/B/C, CCL12, CCL2, CCL3, CCL4, CCNB2, CD3G, CD63, CD74, CD81, CD9, CST7, CTSB, CXCL10, CXCL2, FTL1, H2-AA, H2-AB1, H2AFV, H2AFZ, H2-D1, H2-DMA, H2-DMB1, H2-DMB2, H2-EB1, H2-K1, H2-OA, H2-OB, H2-Q10, H2-Q4, H2-Q6, H2-Q7, H2-T23, HEXB, HMGB2, HMGN2, IFI204, IFI2712A, IFIT3, IFITM3, IL1B, IRF7, ISG15, LGALS3BP, LGMN, LPL, LY6E, MLF, MR1, MRC1, MS4A4B, OASL2, OLFML3, P2RY12, PF4, RTP4, SLFN2, SPARC, STMN1, TMEM119, TUBA1B, TUBB5, or USP18 is higher in the second biological sample from the patient than in the first biological sample from the patient, then the patient is administered one or more additional doses of the TREM2 agonist.

4. A method of monitoring a patient response to a TREM2 agonist, comprising the steps of:
(a) obtaining a first biological sample from the patient prior to administration of the TREM2 agonist to the patient;
(b) measuring a level in the first biological sample from the patient of one or more biomarkers selected from APOE, B2M, BIRC5, BST2, C1QA/B/C, CCL12, CCL2, CCL3, CCL4, CCNB2, CD3G, CD63, CD74, CD81, CD9, CST7, CTSB, CXCL10, CXCL2, FTL1, H2-AA, H2-AB1, H2AFV, H2AFZ, H2-D1, H2-DMA, H2-DMB1, H2-DMB2, H2-EB1, H2-K1, H2-OA, H2-OB, H2-Q10, H2-Q4, H2-Q6, H2-Q7, H2-T23, HEXB, HMGB2, HMGN2, IFI204, IFI2712A, IFIT3, IFITM3, IL1B, IRF7, ISG15, LGALS3BP, LGMN, LPL, LY6E, MLF, MR1, MRC1, MS4A4B, OASL2, OLFML3, P2RY12, PF4, RTP4, SLFN2, SPARC, STMN1, TMEM119, TUBA1B, TUBB5, or USP18;
(c) administering to the patient an effective amount of a TREM2 agonist;
(d) obtaining a one or more subsequent biological samples from the patient after administration of the TREM2 agonist to the patient; and
(e) measuring a level in the subsequent biological sample(s) of one or more biomarkers selected from APOE, B2M, BIRC5, BST2, C1QA/B/C, CCL12, CCL2, CCL3, CCL4, CCNB2, CD3G, CD63, CD74, CD81, CD9, CST7, CTSB, CXCL10, CXCL2, FTL1, H2-AA, H2-AB1, H2AFV, H2AFZ, H2-D1, H2-DMA, H2-DMB1, H2-DMB2, H2-EB1, H2-K1, H2-OA, H2-OB, H2-Q10, H2-Q4, H2-Q6, H2-Q7, H2-T23, HEXB, HMGB2, HMGN2, IFI204, IFI2712A, IFIT3, IFITM3, IL1B, IRF7, ISG15, LGALS3BP, LGMN, LPL, LY6E, MLF, MR1, MRC1, MS4A4B, OASL2, OLFML3, P2RY12, PF4, RTP4, SLFN2, SPARC, STMN1, TMEM119, TUBA1B, TUBB5, or USP18;
wherein the levels of one of more biomarkers in the first biological sample and subsequent biological samples can be compared and changes in one or more of the biomarkers indicate a patient response.

5. The method of any one of claims 1-4, wherein the one or more biomarkers are selected from FTL1, MLF, CD63, LPL, CTSB, CST7, APOE, CCL4, CD9, or CCL3.

6. The method of any one of claims 1-4, wherein the one or more biomarkers are selected from C1QA/B/C, CD81, HEXB, IL1B, LGMN, OLFML3, P2RY12, SPARC, TMEM119, MRC1, PF4, CD3G, or MS4A4B.

7. The method of any one of claims 1-4, wherein the one or more biomarkers are selected from H2AFZ, HMGB2, TUBA1B, HMGN2, H2AFV, IFI2712A, TUBB5, BIRC5, STMN1, or CCNB2.

8. The method of any one of claims 1-4, wherein the one or more biomarkers are selected from CCL12, IFITM3, ISG15, IFIT3, BST2, OASL2, LGALS3BP, RTP4, IFI204, or IRF7.

9. The method of any one of claims 1-4, wherein the one or more biomarkers are selected from H2-K1, H2-Q7, H2-EB1, CD74, H2-AA, H2-D1, H2-AB1, H2-DMA, H2-T23, or LY6E.

10. The method of any one of claims 1-4, wherein the one or more biomarkers are selected from BST2, CCL2, IFI204, IFI2712A, IFIT3, IFITM3, IRF7, ISG15, LGALS3BP, OASL2, RTP4, SLFN2, or USP18.

11. The method of any one of claims 1-4, wherein the one or more biomarkers are selected from B2M, H2-D1, H2-K1, H2-Q10, H2-Q4, H2-Q6, H2-Q7, H2-T23, MR1, CD74, H2-AA, H2-AB1, H2-DMA, H2-DMB1, H2-DMB2, H2-EB1, H2-OA, or H2-OB.

12. The method of any one of claims 1-4, wherein the one or more biomarkers are selected from CCL2, CCL4, CST7, CXCL2, CXCL10, IL1B, or TMEM119.

13. A method of inducing microglial activation in a patient towards specific microglia cell type trajectories, comprising administering to the patient an effective amount of a TREM2 agonist, wherein the microglial activation in the patient is towards:
(a) a disease-associated (DAM) microglia type trajectory;
(b) an interferon-responsive (IFN-R) microglia type trajectory;
(c) a cycling (Cyc-M) microglia type trajectory; and/or
(d) an MHC-II expressing (MHC-II) microglia type trajectory,
wherein the patient is diagnosed with Alzheimer's disease.

14. The method of any one of claims 1-13, wherein the TREM2 agonist is an anti-hTREM2 antibody.

15. The method of claim 14, wherein the anti-hTREM2 antibody comprises a light chain variable region comprising a CDRL1 having an amino acid sequence according to SEQ ID NO:6; a CDRL2 having an amino acid sequence according to SEQ ID NO:7; and a CDRL3 having an amino acid sequence according to SEQ ID NO:8, and a heavy chain variable region comprising a CDRH1 having an amino acid sequence according to SEQ ID NO: 10; a CDRH2 having an amino acid sequence according to SEQ ID NO: 11; and a CDRH3 having an amino acid sequence according to SEQ ID NO: 12.

16. The method of claim 14, wherein the anti-hTREM2 antibody comprises a light chain variable region having an amino acid sequence according to SEQ ID NO: 5, and a heavy chain variable region having an amino acid sequence according to SEQ ID NO: 9.

17. The method of claim 15 or 16, wherein the anti-hTREM2 antibody is an IgG., optionally an IgG₁.

18. The method of any one of claims 15-17, wherein the anti-hTREM2 antibody comprises a kappa light constant region.

19. The method of any one of claims 15-18, wherein the anti-hTREM2 antibody is an IgG₁ comprising a variant constant region having one or more mutations selected from R292C, N297G, V302C, D356E, or L358M, according to EU numbering.

20. The method of any one of claims 15-19, wherein the anti-hTREM2 antibody comprises a light chain having an amino acid sequence according to SEQ ID NO: 13, and a heavy chain variable region having an amino acid sequence according to SEQ ID NO: 16.


SEQUENCE LISTING (18 sequences)

SEQ ID NO: 1 - protein, 230 aa, Artificial Sequence
  Synthetic Polypeptide
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEHSISRSLLEGEIPFPPTSILLLLACIFLIKILAASALWAAAWHGQKPGTHPPSELDCGHDPGYQLQTLPGLRDT

SEQ ID NO: 2 - protein, 173 aa, Artificial Sequence
  Synthetic Polypeptide
MEPLRLLILLFVTELSGAHNTTVFQGVAGQSLQVSCPYDSMKHWGRRKAWCRQLGEKGPCQRWSTHNLWLLSFLRRWNGSTAITDDTLGGTLTITLRNLQPHDAGLYQCQSLHGSEADTLRKVLVEVLADPLDHRDAGDLWFPGESESFEDAHVEHSISRSLLEGEIPFPPTS

SEQ ID NO: 3 - protein, 112 aa, Artificial Sequence
  Synthetic Polypeptide
MGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYY

SEQ ID NO: 4 - protein, 234 aa, Artificial Sequence
  Synthetic Polypeptide
MRKTRLWGLLWMLFVSELRAATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKGFSGTPGSNENSTQNVYKIPPTTTKALCPLYTSPRTVTQAPPKSTADVSTPDSEINLTNVTDIIRVPVFNIVILLAGGFLSKSLVFSVLFAVTLRSFVP

SEQ ID NO: 5 - protein, 107 aa, Artificial Sequence
  Synthetic Polypeptide
EIVMTQSPATLSVSPGERATLSCRASQSVSSNLAWFQQKPGQAPRLLIYGASTRATGIPARFSGSGSGTEFTLTISSLQPEDFAVYYCLQDNNFPPTFGQGTKVDIK

SEQ ID NO: 6 - protein, 11 aa, Artificial Sequence
  Synthetic Polypeptide
RASQSVSSNLA

SEQ ID NO: 7 - protein, 7 aa, Artificial Sequence
  Synthetic Polypeptide
GASTRAT

SEQ ID NO: 8 - protein, 9 aa, Artificial Sequence
  Synthetic Polypeptide
LQDNNFPPT

SEQ ID NO: 9 - protein, 121 aa, Artificial Sequence
  Synthetic Polypeptide
EVQLVQSGAEVKKPGESLKISCKGSGYSFTSYWIGWVRQMPGKGLEWMGIIYPGDADARYSPSFQGQVTISADKSISTAYLQWSSLKASDTAMYFCARRRQGIFGDALDFWGQGTLVTVSS

SEQ ID NO: 10 - protein, 5 aa, Artificial Sequence
  Synthetic Polypeptide
SYWIG

SEQ ID NO: 11 - protein, 17 aa, Artificial Sequence
  Synthetic Polypeptide
IIYPGDADARYSPSFQG

SEQ ID NO: 12 - protein, 12 aa, Artificial Sequence
  Synthetic Polypeptide
RRQGIFGDALDF

SEQ ID NO: 13 - protein, 214 aa, Artificial Sequence
  Synthetic Polypeptide
EIVMTQSPATLSVSPGERATLSCRASQSVSSNLAWFQQKPGQAPRLLIYGASTRATGIPARFSGSGSGTEFTLTISSLQPEDFAVYYCLQDNNFPPTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 236 aa, Artificial Sequence
  Synthetic Polypeptide
MDMRVPAQLLGLLLLWLRGARCEIVMTQSPATLSVSPGERATLSCRASQSVSSNLAWFQQKPGQAPRLLIYGASTRATGIPARFSGSGSGTEFTLTISSLQPEDFAVYYCLQDNNFPPTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 214 aa, Artificial Sequence
  Synthetic Polypeptide
EIVMTQSPATLSVSPGERATLSCRASQSVSSNLAWFQQKPGQAPRLLIYGASTRATGIPARFSGSGSGTEFTLTISSLQPEDFAVYYCLQDNNFPPTFGQGTKVDIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 16 - protein, 451 aa, Artificial Sequence
  Synthetic Polypeptide
EVQLVQSGAEVKKPGESLKISCKGSGYSFTSYWIGWVRQMPGKGLEWMGIIYPGDADARYSPSFQGQVTISADKSISTAYLQWSSLKASDTAMYFCARRRQGIFGDALDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 473 aa, Artificial Sequence
  Synthetic Polypeptide
MDMRVPAQLLGLLLLWLRGARCEVQLVQSGAEVKKPGESLKISCKGSGYSFTSYWIGWVRQMPGKGLEWMGIIYPGDADARYSPSFQGQVTISADKSISTAYLQWSSLKASDTAMYFCARRRQGIFGDALDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 445 aa, Artificial Sequence
  Synthetic Polypeptide
EVQLVQSGAEVKKPGESLKISCKGSGYSFTSYWIGWVRQMPGKGLEWMGIIYPGDADARYSPSFQGQVTISADKSISTAYLQWSSLKASDTAMYFCARRRQGIFGDALDFWGQGTLVTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK